(19) United States
(12) Patent Application Publication
(10) Pub. No.: US 2025/0388920 A1
(43) Pub. Date: Dec. 25, 2025

(54) GENETICALLY MODIFIED PLANTS HAVING INCREASED OIL AND OLEIC ACID CONTENT AND METHODS OF PRODUCING SAME

(71) Applicant: East Tennessee State University Research Foundation, Johnson City, TN (US)

(72) Inventors: Aruna Kilaru, Johnson City, TN (US); Jyoti Behera, Johnson City, TN (US)

(73) Assignee: East Tennessee State University Research Foundation, Johnson City, TN (US)

(21) Appl. No.: 19/244,022

(22) Filed: Jun. 20, 2025

Related U.S. Application Data
(60) Provisional application No. 63/662,664, filed on Jun. 21, 2024.

(51) Int. Cl. C12N 15/82; C07K 14/415

(52) U.S. Cl. CPC C12N 15/8247; C07K 14/415; C12N 15/8205

(57) ABSTRACT

Disclosed herein is a method for producing a lipid or oil in a plant, the method comprising genetically modifying the plant to express a plurality of heterologous proteins selected from PaWRI1, PaWRI2, PaDGAT1, or PaPDAT1, or variants thereof. The expression of the plurality of heterologous proteins in the genetically modified plant may result in a change in the nutrient profile of the plant relative to non-genetically modified plants of the same species. Also disclosed herein is a method for producing fatty acid and triacylglycerol content in plant non-seed tissue. Further disclosed herein is a method for producing a genetically modified plant.

### CROSS-REFERENCE TO RELATED APPLICATIONS

The present application claims priority to U.S. Provisional Application No. 63/662,664, filed Jun. 21, 2024, the entirety of which is incorporated herein by reference.

### STATEMENT REGARDING FEDERALLY SPONSORED RESEARCH

This invention was made with government support under Grant 13058738 awarded by the U.S. Department of Agriculture's (USDA) National Institute of Food and Agriculture (NIFA). The government has certain rights in the invention.

### SEQUENCE LISTING

The instant application contains a Sequence Listing which has been submitted electronically in XML format and is hereby incorporated by reference in its entirety. Said XML copy, created on Jun. 19, 2025, is named “ETU0010.xml” and is 168605 bytes in size.

### TECHNICAL FIELD

The present disclosure relates generally to the field of genetically modified plants, and more specifically to methods of producing genetically modified plants to increase oil and/or lipid content.

### BACKGROUND

The quest for the alternate source of vegetable oil and biofuel production has been a priority to meet the increasing demand of the growing population worldwide. Although most of the plant oils are derived from the seeds, they vary in their fatty acid composition, making them undesirable for human consumption. Avocado fruit mesocarp (non-seed tissue), on the other hand, synthesizes and stores copious amount of heart-healthy oleic-acid (C18:1) rich oil, predominantly as triacylglycerol.

Plants synthesize and store oil primarily as triacylglycerol (TAG), high-energy molecules found in both seed and non-seed tissues. In seeds, the breakdown of TAGs provides the main source of energy and carbon during germination, while in non-seed parts of the plant, TAGs serve various functions. Plant oils are also crucial for human and animal nutrition, the oil industry, and as renewable energy sources. Vegetable oil consumption constitutes 25% of human dietary calories, and its demand is expected to double by 2030. Additionally, plant oils have significant potential applications in biofuel production, offering an alternative to lignocellulosic-based fuels such as ethanol.

The quest for sustainable energy sources has gained significant attention due to growing population, concerns over climate change, and limited fossil fuel resources. Vegetable oils have potential applications in biofuel production that can be used as an alternative to lignocellulosic-based fuel such as ethanol. However, the composition of plant oils often does not meet the requirements of specific applications, necessitating genetic engineering approaches to enhance desired traits.

Oleic acid, a monounsaturated FA, possesses numerous desirable characteristics, including high oxidative stability, low viscosity, and improved nutritional properties. Moreover, oleic acid-rich oils have been associated with potential health benefits, including immune enhancement, anti-inflammatory, anti-cancer, and antioxidant properties. It also lowers low-density lipoprotein (bad cholesterol) and improves high-density lipoprotein (good cholesterol), reducing hypertension risk. Beyond nutritional benefits, oleic acid is used in pharmaceuticals, cosmetics, and personal care products due to its emulsifying, lubricating, and excipient properties.

The highly complex acyl-lipid biosynthesis process in plants comprises almost 600 genes acting on 120 different enzymatic reactions in non-linear and interconnected metabolic networks. The intricate nature of lipid biosynthesis, the diversity of enzymes, and the need to consider multiple criteria contribute to the difficulty in deciphering lipid biosynthesis pathways.

Accordingly, a continual need exists to elucidate the roles of various genes and proteins in the biosynthesis process and to develop methods and techniques to genetically engineer plants to have desired traits based on these discoveries.

### SUMMARY

The present disclosure elucidates the regulatory roles of PaWRI1 (SEQ ID NO: 2) and PaWRI2 (SEQ ID NO: 4) in oil biosynthesis and lipid metabolism in non-seed mesocarp tissue and demonstrates the feasibility in applying these findings to enhance oleic acid (C18:1) rich triacylglycerol (TAG) content in other plants. For example, the instant disclosure elucidates a functional role for WRI2 (SEQ ID NO: 3) in avocado, a basal angiosperm species, and this function is not conserved in most modern angiosperms and thus provides basis for mechanistic differences in the transcriptional regulation of lipid biosynthesis among different plant species and among various tissues. Further, the instant disclosure demonstrates that avocado WRI1 (SEQ ID NO: 1) and WRI2 is capable of transactivation of fatty acid biosynthesis genes and TAG accumulation, synergistically with DGAT1 (SEQ ID NO: 5) and PDAT1 (SEQ ID NO: 7), in non-seed tissues.

Disclosed herein is a method for producing a lipid or oil in a plant, the method comprising genetically modifying the plant to express a plurality of heterologous proteins selected from PaWRI1 (SEQ ID NO: 2), PaWRI2 (SEQ ID NO: 4), PaDGAT1 (SEQ ID NO: 6), or PaPDAT1 (SEQ ID NO: 8), or variants thereof. The expression of the plurality of heterologous proteins in the genetically modified plant may result in a change in the nutrient profile of the plant relative to non-genetically modified plants of the same species.

Also disclosed herein is a method for producing fatty acid and triacylglycerol content in plant non-seed tissue, the method comprising genetically modifying the plant to express a plurality of heterologous proteins in the plant non-seed tissue, the heterologous proteins selected from PaWRI1 (SEQ ID NO: 2), PaWRI2 (SEQ ID NO: 4), PaDGAT1 (SEQ ID NO: 6), or PaPDAT1 (SEQ ID NO: 8), or variants thereof. The expression of the plurality of heterologous proteins may result in a change in the nutrient profile of the plant relative to non-genetically modified plants of the same species.

Further disclosed herein is a method for producing a genetically modified plant comprising genetically modifying the plant by one or more of Agrobacterium-mediated transformation, biolistic bombardment, protoplast transformation, electroporation, microinjection, PEG-mediated transformation, CRISPR-Cas9, transgrafting, RNA interference, virus-induced gene silencing, and sonication. The genetically modified plant may express a plurality of heterologous proteins selected from PaWRI1 (SEQ ID NO: 2), PaWRI2 (SEQ ID NO: 4), PaDGAT1 (SEQ ID NO: 6), or PaPDAT1 (SEQ ID NO: 8), or variants thereof. The expression of the plurality of heterologous proteins may result in a change in the nutrient profile of the genetically modified plant relative to non-genetically modified plants of the same species.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIGS. 1A-1E demonstrate that the co-expression of WRI1+WRI2+P19 in N. benthamiana increased the fatty acid levels in leaves through upregulating the genes involved in the biosynthetic pathways in a distinct manner from WRI1+P19 or WRI2+P19 alone. FIGS. 1A-1E depict the quantification of LD, the total lipid and fatty acid content, and the fatty acid profile in N. benthamiana leaves co-expressing PaWRI1 and PaWRI2. FIG. 1A depicts the number of accumulated LDs per unit area and FIG. 1B their average size (area). FIG. 1C depicts the quantity of total lipid extracted from the leaves. FIG. 1D depicts the total fatty acid content. FIG. 1E depicts the fatty acid profile of the extracted total lipids. Values represent mean±SD of six independent experiments and different letters indicate significant differences (P<0.05), as determined by one-way analysis of variance (ANOVA) with Tukey's post-test.

FIGS. 2A-2D demonstrate the combined effect of PaDGAT1 and PaPDAT1 on TAG biosynthesis in non-seed tissue. FIG. 2A depicts the quantification of total lipid extracted from the leaves and separation of TAG by TLC. FIG. 2B graphically depicts the quantity of accumulated LDs per unit area and FIG. 2C depicts their average size. FIG. 2D depicts the FA profile of extracted TAG from the TLC plates. Values represent average and SD of three independent experiments and different letters indicate significant differences (P<0.05), as determined by one-way analysis of variance (ANOVA) with Tukey's post-test.

FIGS. 3A-3E demonstrate the impact of multigene expression for fatty acid content and composition in N. benthamiana leaves. FIG. 3A depicts the number of accumulated lipid droplets per unit area and FIG. 3B depicts their average size (area). FIG. 3C depicts the quantity of total lipid extracted from the leaves. FIG. 3D depicts the total fatty acid content. FIG. 3E depicts the fatty acid profile of the extracted total lipids. Values represent mean±SD of six independent experiments and different letters indicate significant differences (P<0.05), as determined by one-way analysis of variance (ANOVA) with Tukey's post-test.

FIGS. 4A-4E depict the quantification of lipid droplets, total lipid and fatty acid content, and FA profile in multigene ‘push-pull’ co-expression. FIG. 4A depicts the number of accumulated LDs per unit area and FIG. 4B depicts their average size (area). FIG. 4C depicts the quantity of total lipid extracted from the leaves. FIG. 4D depicts the total FA content. FIG. 4E depicts the FA profile. Values represent mean±SD of six independent experiments and different letters indicate significant differences (P<0.05), as determined by one-way analysis of variance (ANOVA) with Tukey's post-test.

FIG. 5 depicts the analysis of TAG content in N. benthamiana leaves co-expressing avocado genes. Values represent mean±SD of three independent experiments and different letters indicate significant differences (P<0.05), as determined by one-way analysis of variance (ANOVA) with Tukey's post-test.

FIGS. 6A-6E depict the FA profile of TAG. FIG. 6A depicts palmitic acid (16:0). FIG. 6B depicts stearic acid (18:0), FIG. 6C depicts oleic (18:1); FIG. 6D depicts linoleic acid (18:2) FIG. 6E depicts alpha-linolenic acid (18:3) Values represent mean±SD of six independent experiments and different letters indicate significant differences (P<0.05), as determined by one-way analysis of variance (ANOVA) with Tukey's post-test.

FIGS. 7A-7D graphically depict the qRT-PCR analysis of lipid biosynthesis genes, including glycolysis (FIG. 7A), FA-biosynthesis (FIG. 7B), TAG assembly (FIG. 7C), and regulatory genes (FIG. 7D) in N. benthamiana leaves expressing P19 (control), P19+PaWRI1, P19+PaWRI2, and P19+PaWRI1+PaWRI2 was done using gene specific primers. Data represents mean values of gene expression relative to L23 housekeeping gene.

FIG. 8 depicts a heat map of relative expression values of glycolysis, FA biosynthesis and TAG assembly genes in N. benthamiana leaves.

FIG. 9 depicts a Y1Hassay showing +ve and −ve controls, and the transactivation of PaWRI2-AW-box by PaWRI1 and PaWRI2.

FIG. 10 depicts a schematic representation of AW-box elements identified in avocado gene promoters.

FIGS. 11A-11E depict the lipid content and fatty acid composition of N. benthamiana leaves transiently expressing C-terminal deletion constructs of PaWRI1. FIG. 11A depicts the quantification of accumulated LDs per unit surface area of the leaf tissue. FIG. 11B depicts the average size of accumulated LDs. FIG. 11C depicts the quantification of total lipid (TL) content. FIG. 11D depicts the quantification of the fatty acid content. FIG. 11E depicts the fatty acid profile of total lipids extracted from the infiltrated leaves. Data represents mean+sd (n=6; p<0.05).

FIGS. 12A-12E depict the lipid content and fatty acid composition of N. benthamiana leaves transiently expressing C-terminal deletion constructs of PaWRI2. FIG. 12A depicts the quantification of accumulated LDs per unit surface area of the leaf tissue. FIG. 12B depicts the average size of accumulated LDs. FIG. 12C depicts the quantification of total lipid (TL) content. FIG. 12D depicts the quantification of the fatty acid content. FIG. 12E depicts the fatty acid profile of total lipids extracted from the infiltrated leaves. Data represents mean+sd (n=6; p<0.05).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As described herein, the present disclosure relates to genetically modified plants and methods for producing genetically modified plants. In some embodiments, the disclosure relates to methods of genetically engineering plants to alter the nutrient profile. In some embodiments, the disclosure relates to methods of producing heterologous proteins in plants. In some embodiments, the disclosure relates to producing lipids and oils in plants.

Plants synthesize and store oil, mostly as triacylglycerol (TAG), in the form of high energy molecules in their seed and non-seed tissues. The total biomass of non-seed portions of a plant is much higher compared to the biomass of the seed tissues, increasing oil bioproduction in the non-seed tissues, by even small margin, may lead to an increase in total oil production. Accordingly, in some embodiments, the present disclosure is related to genetically modified plants that have been engineered to increase oil production. Optionally, the genetically modified plants have been engineered to increase oil production in non-seed tissues.

The present disclosure elucidates the regulatory roles of PaWRI1 and PaWRI2 in oil biosynthesis and lipid metabolism in non-seed mesocarp tissue and demonstrates the feasibility in applying these findings to enhance oleic acid (C18:1) rich triacylglycerol (TAG) content in other plants.

Specifically, the present disclosure demonstrates that multigene transient overexpression of PaWRI1, PaWRI2, PaDGAT1, and PaPADT1 in N. benthamiana leaves resulted in increased lipid biosynthesis, lipid droplet size, and C18:1 content. Further, PaWRI1 and PaWRI2 selectively transactivated plastidial glycolytic enzymes and upregulated fatty acid (FA) genes, indicating a role in lipid metabolism regulation.

As used herein, the term “CX:D” in reference to a fatty acid refers to the lipid number where X is the number of carbon atoms in the fatty acid and D is the number of double bonds. Accordingly, the expression “C18:1” refers to a fatty acid having 18 carbon atoms and 1 double bond.

In some embodiments, PaWRI2 transactivates fatty acid biosynthesis genes and promotes TAG accumulation in non-seed tissues, including synergizing with other enzymes, such as DGAT1 and PDAT1. Without being bound by theory, the present disclosure elucidates a functional role for WRI2 in a basal angiosperm species, a role likely lost in modern angiosperms, providing insights into the mechanistic differences in the transcriptional regulation of lipid biosynthesis among different plant species and between seed and non-seed tissues.

In some embodiments, the present disclosure relates to a method for producing a lipid or oil in a plant, by genetically modifying the plant to express a plurality of heterologous proteins wherein expression of the plurality of heterologous proteins results in a change or alteration in the nutrient profile of the plant relative to non-genetically modified plants of the same species, as further described herein.

As used herein, “genetically modified” refers to a plant whose genetic material has been altered using genetic engineering techniques. Any suitable method of genetically modifying the plant as known in the art is contemplated and possible. For example, and without being bound by theory, the plants can be genetically modified using one or more of Agrobacterium-mediated transformation, biolistic (particle) bombardment, protoplast transformation, electroporation, microinjection, PEG-mediated transformation, CRISPR-Cas9, transgrafting, RNA interference, virus-induced gene silencing, sonication and the like.

In some embodiments, the plant that is genetically modified may be a commercial crop. In some embodiments, the plant may be a commercial source of vegetable oil. Exemplary, non-limiting plants suitable for use with the methods disclosed herein include canola (Brassica sp. such as Brassica carinata, Brassica juncea, Brassica napobrassica, Brassica napus) mustard (Brassica juncea), other Brassica (e.g., Brassica napobrassica, Brassica camelina), sunflower (Helianthus sp. such as Helianthus annuus), linseed (Linum usitatissimum), soybean (Glycine max), safflower (Carthamus tinctorius), corn (Zea mays), tobacco (Nicotiana sp, such as Nicotiana tabacum or Nicotiana benthamiana), peanut (Arachis hypogaea), palm (Elaeis guineensis), cottonseed (Gossypium hirsutum), coconut (Cocos nucifera), avocado (Persea americana), olive (Olea europaea), cashew (Anacardium occidentale), macadamia (Macadamia intergrifolia), almond (Prunus amygdalus), oat (Avena sativa), rice (Oryza sp, such as Oryza sativa and Oryza glaberrima), Arabidopsis (Arabidopsis thaliana), Acrocomia aculeata (macauba palm), Astrocaryum murumuru (murumuru), Beta vulgaris (sugar beet), Camelina sativa (false flax), Caryocar brasiliense (pequi), Crambe abyssinica (Abyssinian kale), Cucumis melo (melon), Hordeum vulgare (barley), Jatropha curcas (physic nut), Ricinus communis (castor), Saccharum sp. (sugarcane), Sesamum indicum (sesame), Solanum tuberosum (potato), Sorghum sp, such as Sorghum bicolor, Sorghum vulgare, Triticum sp. (wheat) such as Triticum aestivum, combinations thereof, and the like, though any oil producing plant is contemplated and possible.

In some embodiments, the plant may be an oilseed plant, such as an oilseed crop plant. As used herein, an “oilseed plant” refers to a plant species used for the commercial production of lipid from the seeds of the plant. Exemplary, non-limiting oilseed plants include oil-seed rape (e.g., canola), maize, sunflower, safflower, soybean, sorghum, flax (linseed), sugar beet, Brassicas, cotton, peanut, poppy, rutabaga, mustard, castor bean, sesame, safflower, nut-producing plants. The oil-seed plant may optionally also produce oil from non-seed tissue, such as leaves, stems, tubers, roots, mesocarp of a fruit, and the like.

In some embodiments, the plant may produce high levels of lipid in its fruit such as olive, oil palm, avocado, coconut, peach palm, or sea buckthorn. In some embodiments, the plant may be a horticultural plant, such as, tubers, fruits or vegetables. The plant may optionally produce oil in non-seed tissue, such as leaves, stems, tubers, roots, mesocarp of a fruit and the like, and may optionally also produce oil in seeds.

In some embodiments, the genetically modified plant may be used for cellular agriculture. As used herein, “cellular agriculture” refers to the production of agricultural products from cell cultures. In embodiments, the cellular agriculture may include microbial systems, such as bacteria, fungi, and the like, or photosynthetic systems, such as mosses, algae, and the like, to produce oil.

In some embodiments, the genetically modified plant may be engineered to produce one or more heterologous proteins. In some embodiments, the genetically modified plant is engineered to produce a plurality of heterologous proteins, including 2, 3, 4, 5, 6, 7, 8, 9, 10, or more heterologous proteins. As used herein, “heterologous” refers to a biomolecule that is produced by an organism as a result of introducing genetic material from another species or source into its genome. The introduced genetic material contains the instructions for producing a heterologous protein. The protein is considered heterologous because it is not normally produced by the organism's own genome but rather by the inserted genetic material.

In some embodiments, the heterologous protein may regulate oil biosynthesis. It will be appreciated that oil biosynthesis includes three overarching steps. Fatty acid synthesis occurs in plastids and involves elongation and desaturation of fatty acids. Triacylglycerol (TAG) assembly occurs in the endoplasmic reticulum after the fatty acids are converted to acyl-CoA derivatives and enter the glycerolipid pathway (Kennedy Pathway). After assembly, TAG is stored in lipid droplets for energy reserves and other functions. In some embodiments, the heterologous protein may regulate fatty acid synthesis and/or the Kennedy Pathway and/or storage of oils.

In some embodiments, the heterologous protein may be a homolog to a native protein that regulates oil biosynthesis. As used herein, “homolog” refers to a gene, protein, or other biological molecule that shares a common ancestry with a gene, protein, or structure in another organism. Homologs are generally derived from a common ancestral sequence and may be classified into different types based on their evolutionary relationships and functional divergence. As used herein, “homolog” encompasses orthologs, paralogs, and/or xenologs. In some embodiments, the heterologous protein is an ortholog to a native protein that regulates oil biosynthesis.

In some embodiments, the heterologous protein may be a transcription factor that regulates oil biosynthesis. In some embodiments, the heterologous protein may be a transcription factor that demonstrates a high expression level of its encoding gene(s) in the mesocarp of one or more plants.

Exemplary transcription factors include, but are not limited to, WRI1 (WRINKLED1), WRI2 (WRINKLED2), WRI3 (WRINKLED3) (SEQ ID NO: 146), WRI4 (WRINKLED4) (SEQ ID NO: 147), LEC1 (LEAFY COTYLEDON1, LEC2 (LEAFY COTYLEDON2), FUS3 (FUSCA3), ABI3 (ABSCISIC ACID-INSENSITIVE3), ODD1 (OBESUM DULCIS DICOT1), APETALA2/ETHYLENE RESPONSE FACTOR (AP2/ERF), AtMYB89, bZIP67, GL2 (GLABRA2), MYB transcription factors, DOF transcription factors, combinations thereof, and the like.

In some embodiments, the heterologous protein is a functionally-equivalent variant of the transcription factor. In some embodiments, a functionally-equivalent variant has at least 70%, at least 75%, at least 80%, at least 85%, at least 90%, at least 95%, at least 96%, at least 97%, at least 98%, at least 99%, or 100% sequence identity with the transcription factor. In some embodiments, a functionally-equivalent variant maintains binding affinity and specificity with the AW-box or variants thereof (e.g., AWL1, AWL2) located in the promoter of a target gene.

As used herein, the “AW-box” refers to a specific DNA sequence motif that transcription factors can recognize and bind to in the promoters of genes. The binding of transcription factors to such motifs typically regulates the expression of associated genes. For example, and without being bound by theory, the conserved nucleotide motif may be the canonical “CnTnG(n)7CG” (where n represents any nucleotide). In some embodiments, the AW-box is the AWL1 box, having a conserved nucleotide motif of “CG(n)6CnAnG.” In some embodiments, the AW-box is the AWL2 box, having a conserved nucleotide motif of “G(n)7CnAnG.”

In some embodiments, the heterologous protein may be a C-terminally modified variant of the transcription factor. Optionally, the C-terminally modified variant may be a truncated variant of the transcription factor. In some embodiments, the C-terminally modified variant includes the deletion of a C-terminally-located ordered region from the transcription factor. In some embodiments, the C-terminally modified variant maintains binding affinity and specificity with the AW box or variants thereof (e.g., AWL1, AWL2) located in the promoter of a target gene. In some embodiments, genetic engineering of avocado WRI2 through strategic modification, including deletion of a select region in the C-terminus may further improve the transactivation capability with improved effect on lipid production.

In some embodiments, the genetically modified plant may be engineered to produce an ortholog of WRI1. That is, in some embodiments, the heterologous protein may be an ortholog of WRI1. In some embodiments, the heterologous protein may be a functionally equivalent variant of WRI1. In some embodiments, the heterologous protein may be a C-terminally modified variant of WRI2.

Optionally, the genetically modified plant is engineered to produce an avocado ortholog of WRI1, referred to herein as PaWRI1. That is, in some embodiments, the heterologous protein may be PaWRI1. In some embodiments, the heterologous protein may be a functionally-equivalent variant of PaWRI1. In some embodiments, the heterologous protein may be a C-terminally modified variant of PaWRI1. Optionally, the C-terminally modified variant of PaWRI1 may be PaWRI1³⁷⁵ (SEQ ID NO: 9). In some embodiments, the C-terminally modified variant of PaWRI1 may be PaWRI1³¹³ (SEQ ID NO: 10). In some embodiments, the C-terminally modified variant of PaWRI1 may be PaWRI1²⁷³ (SEQ ID NO: 11).

In some embodiments, the genetically modified plant may be engineered to produce an ortholog of WRI2. That is, in some embodiments, the heterologous protein may be an ortholog of WRI2. In some embodiments, the heterologous protein may be a functionally equivalent variant of WRI2. In some embodiments, the heterologous protein may be a C-terminally modified variant of WRI2.

Optionally, the genetically modified plant may be engineered to produce an avocado ortholog of WRI2, referred to herein as PaWRI2. That is, in some embodiments, the heterologous protein may be PaWRI2. In some embodiments, the heterologous protein may be a functionally-equivalent variant of PaWRI2. In some embodiments, the heterologous protein may be a C-terminally modified variant of PaWRI2. Optionally, the C-terminally modified variant of PaWRI2 may be PaWRI2³⁸¹ (SEQ ID NO; 12). In some embodiments, the C-terminally modified variant of PaWRI2 may be PaWRI2²⁶⁶ (SEQ ID NO; 13).

In some embodiments, the genetically modified plant may be engineered to produce an ortholog of WRI1 and an ortholog of WRI2 or variants thereof. Optionally, the variants include functionally-equivalent variants and/or C-terminally modified variants.

Optionally, the genetically modified plant may be engineered to produce PaWRI1 and PaWRI2 or variants thereof. That is, in some embodiments, the heterologous proteins may be PaWRI1 and PaWRI2. In some embodiments, the heterologous proteins may be functionally equivalent variants of PaWRI1 and PaWRI2. In some embodiments, the heterologous proteins may be PaWRI1 or a functionally equivalent variant thereof and PaWRI2 or a functionally equivalent variant thereof.

In some embodiments, the heterologous protein may be an enzyme that regulates oil biosynthesis. Optionally, the enzyme may regulate one or more steps in fatty acid synthesis, TAG assembly, or oil storage. Exemplary enzymes include, but are not limited to, acetyl-CoA carboxylase, fatty acid synthase, stearoyl-ACP desaturase, acyl-CoA synthetase, glycerol-3-phosphate acyltransferase, phospholipid:diacylglycerol acyltransferases, lysophosphatidic acid acyltransferase, phosphatidic acid phosphatase, diacylglycerol acyltransferase, oleosins, combinations thereof, and the like.

In some embodiments, the enzyme may use oleic acid as a substrate. Optionally, the enzyme may catalyze one or more steps in TAG assembly. In some embodiments, the enzyme may catalyze the final step of TAG assembly, contributing to oil accumulation.

In some embodiments, the enzyme may be a diacylglycerol acyltransferase 1 (DGAT1) ortholog. Optionally, the DGAT1 may be an avocado DGAT1, referred to herein as PaDGAT1. In some embodiments, the enzyme may be a phospholipid: diacylglycerol acyltransferases 1 (PDAT1) ortholog. Optionally, the PDAT1 may be an avocado PDAT1, referred to herein as PaPDAT1. In some embodiments, the genetically modified plant may be engineered to produce PaDGAT1 and PaPDAT1.

In some embodiments, the genetically modified plant may be engineered to produce one or more transcription factors and one or more enzymes that regulate oil biosynthesis. In some embodiments, the genetically modified plant may be engineered to produce one or more of PaWRI1 and PaWRI2 or variants thereof and one or more of PaDGAT1 and PaPDAT1.

Optionally, the genetically modified plant may be engineered to produce PaWRI1 or variants thereof, PaWRI2 or variants thereof, PaDGAT1 and PaPDAT1. As will be demonstrated herein, co-expression of a plurality of heterologous proteins may result in increased oil biosynthesis.

In some embodiments, production of the one or more heterologous proteins in the genetically modified plant may result in a change in the nutrient profile of the plant relative to non-genetically modified plants of the same species. Plant oils are mostly composed of five common fatty acids, or salts or esters thereof, namely palmitic acid (16:0), stearic acid (18:0), oleic acid (18:1), linoleic acid (18:2) and linolenic acid (18:3), although, depending on the particular species, longer or shorter fatty acids may also be major constituents. These fatty acids differ from each other in terms of acyl chain length and number of double bonds, leading to different physical properties.

As used herein, a “change or alteration in the nutrient profile” of a plant refers to a change at least one of the overall lipid production of the plant or the composition of the lipids produced by the plant. Changes to the nutrient profile of the plant include, but are not limited to, increases or decreases in the quantity of total lipid extracted, increases or decreases in the total fatty acid content, changes in the fatty acid profile (e.g., relative proportions of the fatty acids), and/or increases or decreases in the amount of TAG.

In some embodiments, the production(s) of the heterologous protein results in an increase in the palmitic acid proportion of the lipid content. In some embodiments, the production of the heterologous protein(s) results in an increase oleic acid incorporation in the TAG.

### EXAMPLES

The following Examples are offered by way of illustration and are presented in a manner such that one skilled in the art should recognize are not meant to be limiting to the present disclosure as a whole or to the appended claims.

### Example 1: Metabolic Engineering of Lipid Biosynthesis in Non-seed Tissues to Enhance Oleic Acid Content by Co-Expression of Avocado WRI1, WRI2, DGAT1 and PDAT1

### Materials and Methods

### Plant Material and Growth Conditions

Wild type tobacco (Nicotiana benthamiana) was used for all the transient expression experiments to elucidate the functional role of the studied genes The tobacco seeds were surface sterilized by washing with 70% ethanol (v/v) containing 0.05% v/v Tween-20, followed by washing with 95% ethanol for 2 min each. Then the seeds were washed thoroughly with deionized water and dried. After three days of vernalization at 4° C., the seeds were transferred to soil pots and the plants were grown in a growth chamber (Percival Scientific, Perry, IA, USA) under long day conditions (16 h L/8 h D) at 24° C. and 60% relative humidity. Approximately 6 weeks old plants were used for Agroinfiltration.

Agroinfiltration into Nicotiana benthamiana Leaves

Cloned PaWRI1, PaWRI2, PaDGAT1, and PaPDAT1 in pB110 expression vector and transformed into LBA4404 strain of Agrobacterium were retrieved from a −80° C. freezer and further grown on LB-agar media plates supplemented with rifampicin (25 μg/ml) and kanamycin (50 μg/ml) antibiotics at 28° C. for 2-3 days. One of the grown colonies was picked from each plate and further grown in LB+ Kan liquid media overnight. On the next day, 50 μl of the saturated culture was further inoculated into 5 ml LB media and grown for 6-8 hours.

The grown Agrobacterium cultures were centrifuged at 4000×g for 5 min at room temperature in a swing bucket centrifuge, supernatant was discarded and resuspended in infiltration buffer (5 mM MgSO4, pH 5.7+ 5 mM methyl ethanesulfonate+ 100 mM acetosyringone). The absorbance was measured at 600 nm, further diluted to an OD600 of 0.3 and incubated in dark for 2 hours at room temperature.

Wild type N. benthamiana plants were used for transient expression of all the genes. Leaves of similar size (5-7 cm diameter) from six weeks old plants were used for transient expression. The Agrobacterium infiltration solution harboring the genes and the P19 (SEQ ID NO: 148) were mixed with an equal volume ratio and the P19 alone was used as control in each experiment (Wood et al. A leaf-based assay using interchangeable design principles to rapidly assemble multistep recombinant pathways. PLANT BIOTECHNOL J. 7(9):914-924. doi:10.1111/j.1467-7652.2009.00453.x (2009)). Two leaves in each plant and three such plants (n=6) were used for Agroinfiltration.

All the samples for comparison were randomly infiltrated into different areas in each leaf. The infiltrated areas were marked, and the plants were grown in normal growth conditions for 6-7 days to facilitate gene expression. Leaf samples from the infiltrated areas were harvested and stored at −80° C. until further use.

### Nile Red Staining and Confocal Microscopy to Visualize Lipid Droplets (LDs)

A small portion of the harvested leaf sample was cut out using leaf puncher and used for staining. Nile Red (in dimethyl sulfoxide) was diluted with PIPES buffer (50 mM, pH 7.0) to a final concentration of 2 μg/ml and used for staining of LDs in the leaves. After 20 min of staining, the samples were washed three times (15 min each) with PIPES buffer and leaf discs were immediately observed using a Leica TCS SP8 confocal fluorescence microscope. The excitation wavelength was 488 nm and the emission wavelengths were 560-620 and 640-720 for detection of fluorescence emitted by Nile Red-stained LDs and chlorophyll autofluorescence, respectively. All the images were taken at 40× magnification and 45 z-stacks from 30 μm depth were superimposed to create the final image. The number of LDs were quantified using imageJ software. Three panels from each biological replicate (10⁴ μm²), for a total of nine panels, were analyzed to quantify the LDs. Data were represented as mean±SD.

### Total Lipid and TAG Extraction and FA Profile Analysis

The leaf samples collected 6 days post-infiltration were used for lipid extraction. 50 mg of plant tissue (fresh weight, FW) was homogenized using bead beater (BioSpec Inc., USA) after adding 2 ml isopropanol and then heated at 70° C. for 30 min to inactivate any internal lipase activity. Before homogenization, 50 μg of glyceryl triheptadecanoic acid (tri-17:0) standard (Sigma-Aldrich) was added to the plant tissue. After cooling to room temperature, 1 mL of chloroform and 400 μL water were added to achieve a final ratio of 2 mL isopropanol: 1 mL chloroform: 0.45 ml water. The samples were stored overnight at 4° C. The next day, the supernatant was collected from the sample after vortexing and centrifugation for 5 min at 4000×g. One mL of chloroform and 2 mL of 1M KCl were added to the sample to achieve phase separation. After centrifugation at 1500×g, the upper aqueous phase and interphase were aspirated by using a Pasteur pipette. The aspiration was repeated thrice with 2 mL of 1M KCl and the bottom organic phase was collected and dried under nitrogen gas. Dried lipid samples were reconstituted in 1 mL of chloroform and transferred into a pre-weighed glass vial. After evaporating the chloroform in nitrogen gas, each glass vial was weighed again to obtain the total lipid weights.

The dried lipid samples were dissolved in 100 μl hexane and 50 μl of them were used for FA profile analysis by GC-FID (GC-2010 SHIMADZU). For separation of TAG from the total lipid pool, thin layer chromatography (TLC) was used. Specifically, 30 μl of each lipid sample was spotted onto TLC silica plates (Supelco TLC Silica gel 60 20×20) at equidistant spots and placed in glass solvent tank containing developing solution (hexane: diethyl ether: glacial acetic acid; 80:20:1). After 50 minutes, the TLC plate was dried and the color was developed by exposing it to iodine vapor. The bands corresponding to TAG fraction were scraped using scalpel and transferred to a new glass tube with Teflon cap. FA methyl ester (FAME) derivatives of the fatty acyl species present in the total lipid and TAG were further prepared by adding 2 ml of methanolic HCl solution (1 M) to each sample followed by incubation in 85° C. water bath for 2 hours. After cooling to room temperature, 2 ml hexane and 2 ml KCl (1 M) were added to each sample and vortexed vigorously. The samples were then centrifuged at 4000×g for 10 min to facilitate phase separation and the upper hexane phase containing all the FAMEs was transferred to new vials. After drying in nitrogen vapor, 250 μl of hexane was added to each tube and transferred to the injector vials for gas chromatography. 1 μl of each sample was injected into the column for separation and detection by GC-FID. A capillary column (ZB-FAME; 30 m×0.25 mm I.D., 0.20 μm) with helium as carrier gas (flow rate of 4.6 mL/minute) was used. The injection temperature was set at 160° C., which was ramped after three minutes to 210° C. at 10° C./minute; the detection temperature was 255° C. The retention time for each FA was determined by comparison to the spiked components of a standard FAME mix GLC 63C (Nu-check Prep Inc., USA). FAs and TAG were quantified using a previously described method (Vanhercke T, et al. Synergistic effect of WRI1 and DGAT1 coexpression on triacylglycerol biosynthesis in plants. FEBS LETT. 587(4):364-369. doi:10.1016/j.febslet.2012.12.018 (2013), hereinafter “Vanhercke”) relative to the known quantity of tri-17:0 internal standard added to each sample prior to lipid extraction. 4-6 samples for each test group were used.

### Statistical Analysis

The transient expression data were expressed as their mean value with standard deviation (mean±SD). One-way analysis of variance (ANOVA) followed by Tukey's post-test was performed using Minitab software (version 21) at p-level of 0.05 (p<0.05) to test for significance among the data set and are shown in ‘letter code’ in each figure. Samples having different letter codes are significantly different.

### Results and Discussion

Transient Co-Expression of PaWRI1 and PaWRI2 Further Improved Lipid Content in N. benthamiana Leaves

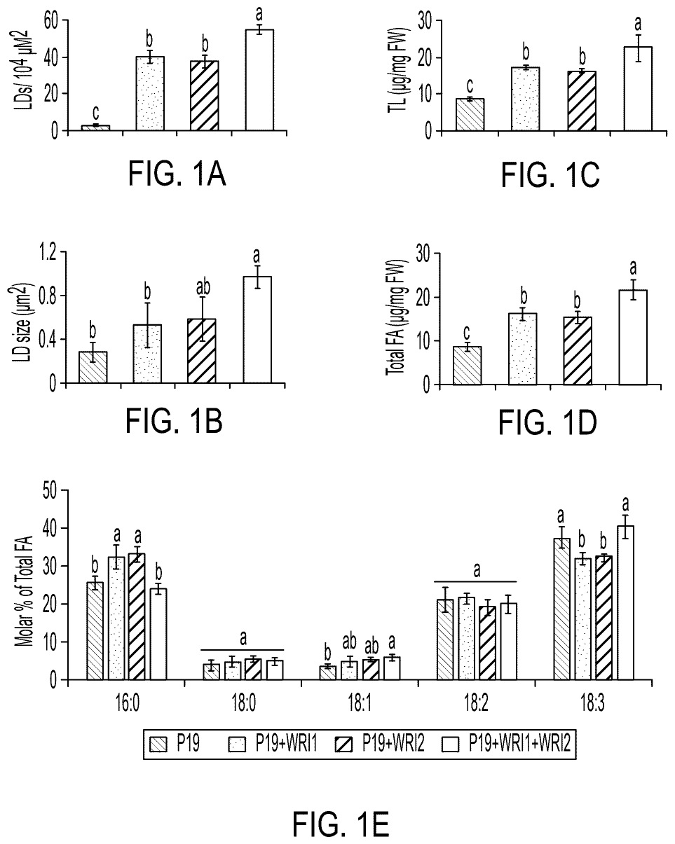

Both PaWRI1 and PaWRI2 were transiently co-expressed in N. benthamiana leaves along with P19 (also used as control). As shown in FIGS. 1A-1E, WRI1+WRI2+P19 co-expression improved the LD accumulation level in the leaves by ˜36% and ˜53% compared to WRI1+P19 and WRI2+P19, respectively (FIG. 1A). These LDs in plants are predominantly composed of TAG and several other class of phospholipids, and thus act as a signature for the lipid metabolic status in the cell.

These results show that co-expression of WRI1, WRI2, and P19 increased the fatty acid (FA) level in leaves by upregulating genes involved in biosynthetic pathways. This effect was distinct from the effects of WRI1+P19 or WRI2+P19 co-expression.

Further, as can be observed in FIG. 1B, an increase in the LD size was observed in the WRI1+WRI2+P19 co-expressing leaves compared to the individual expressions. This suggests a rapid synthesis of TAG in the cell that are packaged into larger LDs. Thus, the increased number and size of LDs observed herein demonstrate an improved storage oil biosynthesis induced by the co-expression.

The total lipid (TL) and FA were also quantified. The results, shown in FIG. 1C and FIG. 1D, demonstrate a statistically significant increase induced by co-expression WRI1+WRI2+P19 compared to individual expression.

While the total lipid content from the PaWRI1, PaWRI2, and P19 leaves showed a 30% and 37% increase compared to PaWRI1+P19 and PaWRI2 +P19 leaves, respectively, the effect was more prominent compared to the P19 control (˜1.6-fold increase) (FIG. 1C). A similar trend was also observed in the FA content, with a 34% and 41% increase in the co-expressed leaves compared to WRI1+P19 and WRI2+P19 leaves, respectively and ˜1.5-fold increase compared to P19 control (FIG. 1D).

Thus, co-expression of PaWRI1 and PaWRI2 in N. benthamiana enhances lipid biosynthesis in plants. Further, the co-expression of PaWRI1 and PaWRI2 in N. benthamiana demonstrates synergistic increases in both total lipid and fatty acid content.

Additionally, co-expression of PaWRI1, PaWRI2, and P19 demonstrated a restorative effect on palmitic acid (C16:0) and linolenic acid (C18:3) in the total lipids extracted. Specifically, as shown in FIG. 1E, PaWRI1, PaWRI2, and P19 co-expression reduced the amount of palmitic acid and increased the amount of linoleic acid back to the levels shown by the P19 control.

Transient Co-Expression of PaDGAT1 and PaPDAT1 Further Improved TAG Production in N. benthamiana Leaves

PaDGAT1 and PaPDAT1 along with P19 were coexpressed in N. benthamiana leaves; P19 expression alone was used as the control.

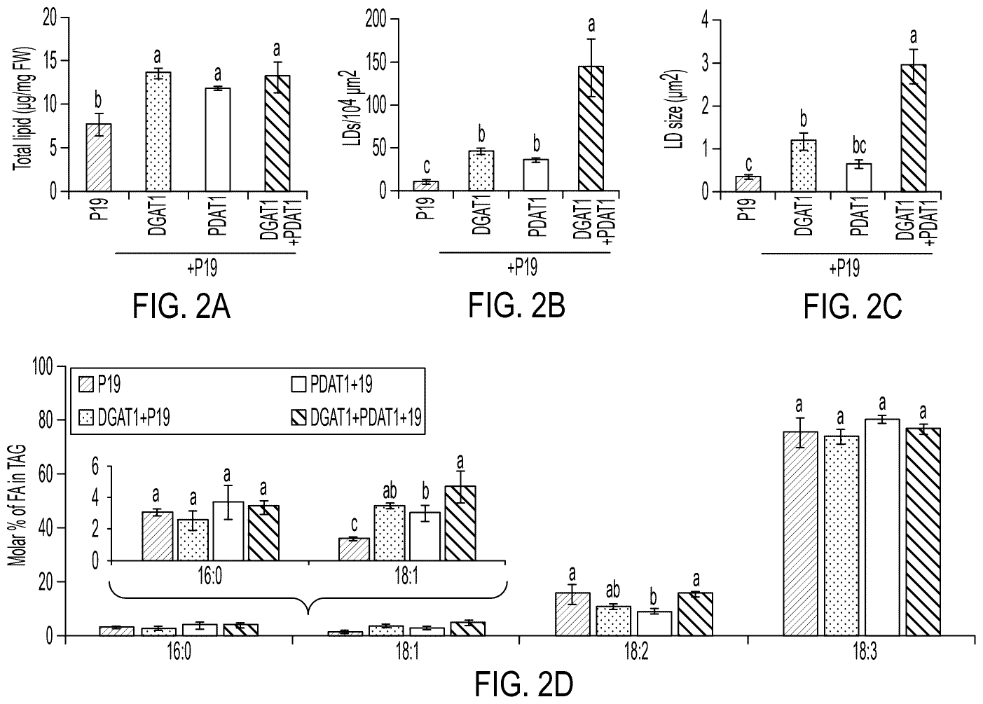

As shown in FIG. 2B, co-expression of PaDGAT1, PaPDAT1, and P19 synergistically increased the lipid droplet accumulation in the leaf tissue by ˜3-fold and ˜4-fold compared to independent expression of PaDGAT1+P19 and PaPDAT1+P19, respectively.

Co-expression also increased average lipid droplet size (area) by greater than 2-fold and greater than 4-fold compared to independent expression of PaDGAT1 and PaPDAT1, respectively, as shown in FIG. 2C, demonstrating improved lipid droplet production and packaging efficiency.

Notably, as shown in FIG. 2A, no statistically significant improvement in total lipid content was observed, suggesting that co-expression does not increase de novo fatty acid biosynthesis but contributes to elevated TAG assembly.

The fatty acid profile of TAG was further analyzed. As shown in FIG. 2A, TAG was separated from the total lipid pool by TLC. TAG was methylated and analyzed by GC-FID, the results of which are shown in FIG. 2D. Co-expression of PaDGAT1, PaPDAT1, and P19 demonstrated an increase in the proportion of oleic acid in the TAG by greater than 30% and greater than 50% compared to the leaves expressing PaDGAT1+P19 and PaPDAT1+P19, respectively.

These results demonstrate that the synchronous activity of PaDGAT1 and PaPDAT1 is responsible for the high proportion of oleic acid in the synthesized TAG, as it was significantly increased (by ˜2.4 fold) compared to the P19 control.

Further, as demonstrated in FIG. 2D, co-expression of PaDGAT1, PaPDAT1, and P19 demonstrated a similar content of polyunsaturated linoleic acid (18:2) in the TAG compared to the control. However, this level was significantly reduced in PDAT1+P19-expressed leaves. It will be appreciated that these results suggest that the co-expression of PaDGAT1 and PaPDAT1 may be a better strategy to specifically improve oleic acid content without affecting other aspects of the fatty acid composition.

Combinatorial ‘Push-Pull’ Strategy Further Improved Lipid Biosynthesis with a Preferential Increase in Oleic Acid

Once the effectiveness of both push (P19+WRI1+WRI2) and pull (P19+DGAT1+PDAT1) approaches in driving lipid accumulation in N. benthamiana leaves was established, the outcomes of the combined ‘push-pull’ strategy were explored. For simplicity, the co-expressions were renamed as P19+WRI1+DGAT1: W1D1; P19+WRI2+DGAT1: W2D1; P19+WRI1+PDAT1: W1P1; P19+WRI2+PDAT1: W2P1.

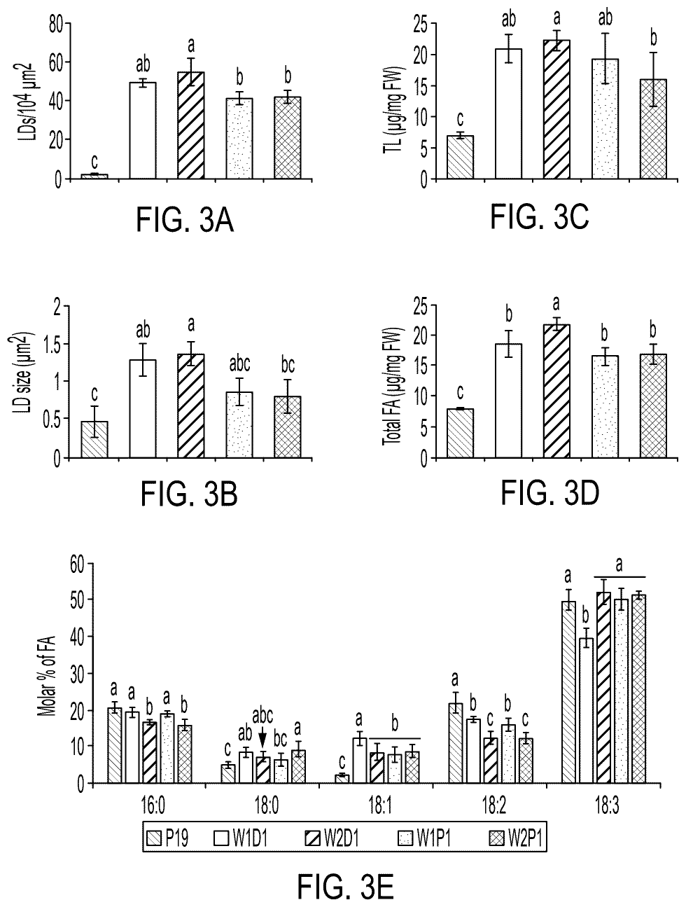

As shown in FIGS. 3A-3E, all transient over-expressions (W1D1, W2D1, W1P1, and W2P1) significantly increased the number of lipid droplets (LDs) in N. benthamiana leaves. Notably, W2D1 showed the most prominent effect, indicating increased storage oil, particularly triacylglycerol (TAG), biosynthesis in the leaves. Moreover, both W1D1 and W2D1 exhibited a greater than 2.5 fold increase in the average LD size, which is usually smaller in non-seed tissues like leaves compared to seeds or fruit mesocarp.

These results suggest enhanced TAG biosynthesis in response to increased neutral lipid synthesis. Assessment of total lipid and fatty acid (FA) content in the leaves showed significant improvements in all over-expressions (FIGS. 3C-3E). Both W1D1 and W2D1 exhibited over a 3-fold increase in total lipid content, indicating that de novo FA biosynthesis induced by PaWRI1 or PaWRI2, combined with the Kennedy pathway of TAG assembly, significantly contributed to lipid biosynthesis. Additionally, the FA profile analysis revealed a significant increase in oleic acid (C18:1) content in all cases compared to the control, with W1D1 showing the most prominent increase. Interestingly, a decrease in C16:0 proportion was observed in W2D1 and W2P1, while all overexpressing leaves showed a significant reduction in C18:2. These results suggest that W1D1 is particularly effective in increasing oleic acid content in leaves.

Multigene Expression of PaWRI1, PaWRI2, PaDGAT1, and PaPDAT1 Further Improved Oleic Acid-Enriched FA Content in N. benthamiana Leaves

The combined expression of PaWRI1 and PaWRI2 resulted in an increased level of C18:1, while co-expression of PaDGAT1 and PaPDAT1 faced limitations due to their inability to induce de novo FA biosynthesis. This led to an investigation of the combined effect of all four genes in N. benthamiana leaf tissues.

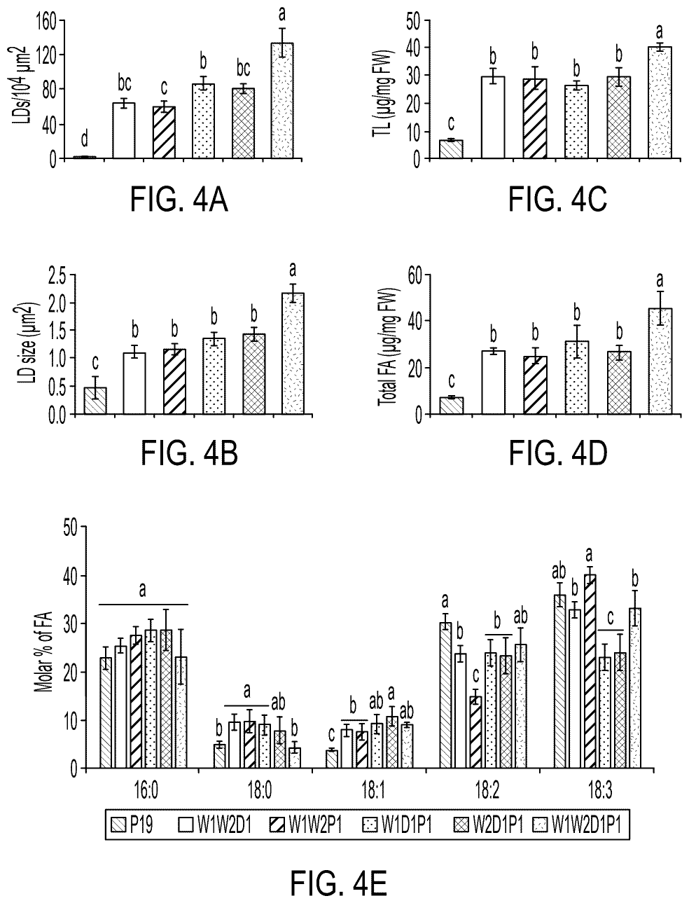

The results showed a staggering >50-fold increase in the lipid droplet number in the W1W2D1P1 leaves, with other combinations also showing >24-fold increase compared to the P19 control (FIG. 4A). This increase was accompanied by an increase in LD sizes, with W1W2D1P1 showing a ˜4-fold increase (FIG. 4B). The average LD size in W1W2D1P1 leaves was similar to that in DGAT1+PDAT1+P19 co-expression leaves (FIG. 4B, FIG. 4C), indicating effective utilization of FA substrates by PaDGAT1 and PaPDAT1 induced by PaWRI1+PaWRI2 to increase TAG biosynthesis.

Moreover, the combined expression of all four genes resulted in the most significant increase (>3-fold) in total lipid content relative to the control leaves (FIG. 4C), accompanied by a net increase in total FA (˜3-fold) (FIG. 4D). While other gene combinations also increased total lipid and FA contents, the combination of WRI1+WRI2 with DGAT1+PDAT1 showed the maximum increase, possibly due to the maximum ‘push’ effect of WRI1+WRI2 combined with the maximum ‘pull’ of DGAT1+PDAT1. Analysis of FA composition in total lipids showed a preferential increase in the C18:1 proportion in leaves expressing all four genes, although W1W2D1, W1W2P1, and W1D1P1 leaf samples also showed a preferential increase in C18:0 at the expense of C18:2 (FIG. 4E). Although no subtle changes were observed in the C16:0 proportions, a decrease in C18:3 levels was observed in W1D1P1 and W2D1P1 leaves (FIG. 4E). Overall, W1W2D1P1 was most effective in increasing lipid accumulation in leaf tissue with a preference for C18:1.

Multigene Expression of PaWRI1, PaWRI2, PaDGAT1 and PaPDAT1 Synergistically Improved the TAG Content in N. benthamiana Leaves

The increase in LD numbers and sizes in all the transient co-expression studies suggested that the avocado genes could enhance TAG content in leaves. To investigate this, the TAG pool was separated from the total lipids by TLC, and their content and FA compositions were measured.

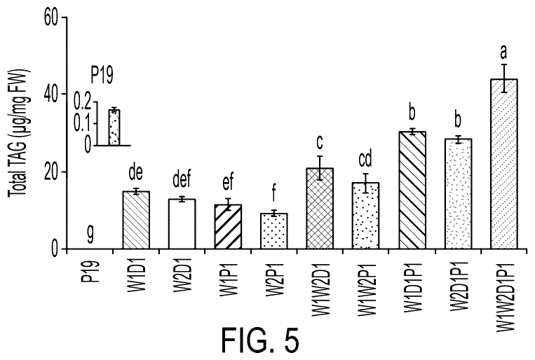

Results showed a high increase in TAG content in leaves in all cases, with W1W2D1P1 showing the highest increase (>260-fold) compared to the P19 control (FIG. 5). Photosynthetic green tissues like leaves typically accumulate very low levels of lipids, of which only 20-30% is TAG. In comparison, W1D1 and W2D1 in our study led to a 90- and 78-fold increase in TAG content, respectively (FIG. 5). All other combined expressions showed higher TAG levels, except W1P1 (˜70-fold) and W2P1 (57-fold). Thus, the combinatorial expression of avocado genes was able to improve TAG content in N. benthamiana leaves, with W1W2D1P1 showing the highest increase. Hence, these genes hold potential for improving TAG content in various seed and non-seed tissues.

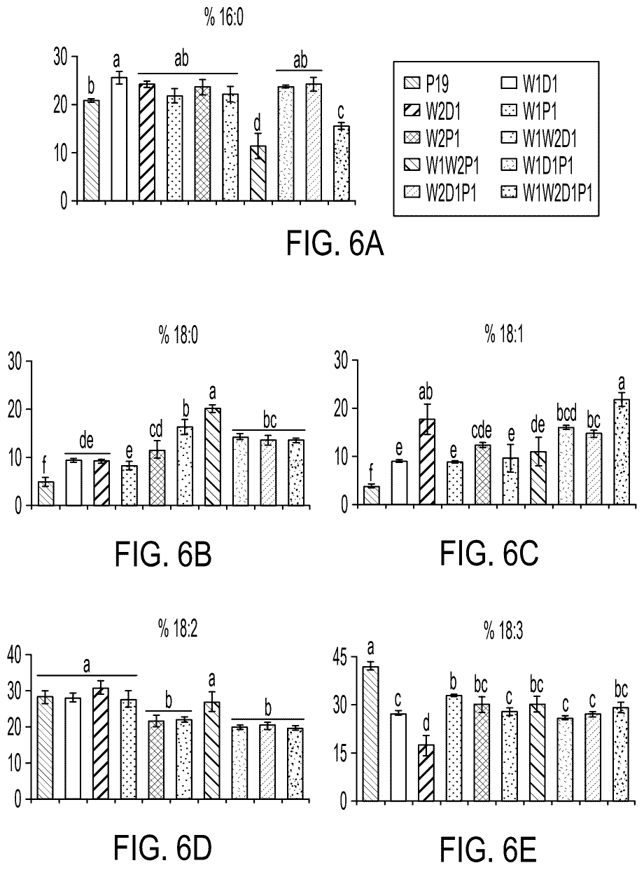

The analysis of FA composition in TAG showed an increase in C18:0 and C18:1 content and a decrease in C18:3 content in all samples (FIG. 6E). While both W2D1 and W1W2D1P1 had a very high proportion of C18:1, the latter showed a higher increase (˜5.6-fold) compared to the P19 control (FIG. 6D); W2D1 showed a ˜4.5-fold increase. While most samples did not show a significant difference in C16:0 levels, W1D1 showed an increase, and W1W2P1 and W1W2D1P1 showed a reduction (FIG. 6A).

An increase in C18:0 in all samples suggests that leaves were able to convert C16:0 to C18:0. Supporting this, desaturation of C18:0 to C18:1 would have occurred, leading to an increase in the C18:1 proportion. However, a reduction in C18:2 was only observed in W2P1, W1W2D1, W1D1P1, and W1W2D1P1, whereas all samples showed reduced C18:3 content.

The elevated C18:1 level in the total lipid and TAG content in the W1W2D1P1 sample in the present study demonstrates that ability of these genes, optionally assisted by additional enzymes like SAD, to further improve oleic acid content in transgenic plants.

### Conclusion

Example 1 thus provides evidence that transient co-expression of avocado genes PaWRI1, PaWRI2, PaDGAT1, and PaPDAT1 substantially improves lipid biosynthesis in N. benthamiana leaves.

The cooperative expression of PaWRI1 and PaWRI2 significantly increased both the number and size of lipid droplets, indicating enhanced oil biosynthesis and storage. This co-expression demonstrated a synergistic effect on lipid accumulation, underscoring the critical roles of PaWRI1 and PaWRI2 in promoting fatty acid and triacylglycerol (TAG) biosynthesis. The observed increase in lipid droplet number and size supports the conclusion that this cooperative expression boosts oil biosynthesis and storage in plant cells.

Furthermore, this study investigates both ‘push’ and ‘pull’ strategies individually and in combination. Both strategies, whether enhancing fatty acid biosynthesis (‘push’) or promoting TAG assembly (‘pull’), effectively elevated lipid content in leaves. The most notable results were achieved with a combined ‘push-pull’ approach, involving the co-expression of PaWRI1, PaWRI2, PaDGAT1, and PaPDAT1. This combined strategy resulted in the highest increase in lipid content and oleic acid (C18:1) composition, demonstrating its potential for biotechnological applications to enhance lipid production across various plant tissues.

### Example 2: Understanding the Mechanistic Role of Avocado WRI1 and WRI2 in Transcriptional Regulation of Plant Oil Biosynthesis

To further understand the regulatory roles exhibited by PaWRI1 and PaWRI2, qRT-PCR analyses were conducted for 47 lipid-synthesis-related genes in N. benthamiana leaves expressing PaWRI1, PaWRI2, or both.

Further, the three-dimensional (3D) structure of both PaWRI1 and PaWRI2 N-terminally located AP2 domains were predicted through in silico analyses and identified their potential target genes in avocado. Further yeast-one-hybrid (Y1H) assay depicted the trans-regulatory roles of both PaWRI1 and PaWRI2 and we evaluated their preference for several target gene promoters. Overall, our work here demonstrates the mechanistic differences in the trans-regulatory roles shown by PaWRI1 and PaWRI2.

### Materials and Methods

### In Silico Analysis

The DNA sequences of the potential target genes in avocado genome were extracted from the avocado genome database using Arabidopsis protein sequences as the query. A tBLASTn (translated nucleotide databases using a protein query) search was performed. The matched sequence showing the highest percentage identity and the lowest e-value was considered orthologous and the corresponding gene sequence, mRNA sequences along with 500 bp upstream of the transcription start site (TSS) were obtained.

The information on the translation initiation site (TIS) (ATG codon) was obtained from the database and the probability was verified using the NetStart-1.0 online tool. The putative WRI1 recognition site AW-box sequence [CnTnG(n)7CG], along with AW-like 1 (AWL1) [GnAnC(n)6GC] and AW-like 2 (AWL2) [GnAnC(n)7G] sequence patterns were searched in the sequences in both forward and reverse direction upstream of the ATG codon using DNA Pattern. The pattern hit sequences along with their orientation and position corresponding to the TIS were obtained and plotted as graphical representations using My Domains Image Creator online tool.

The ab initio prediction of the three-dimensional (3D) structures of PaWRI1, PaWRI2, and AtWRI2 was done using the AlphaFold2 advanced online server. The putative amino acid sequences of PaWRI1 (QXF58928) and PaWRI2 (QXF58929) were obtained from NCBI database and used as input in FASTA format for prediction. The predicted 3D models were downloaded in Protein Data Bank (.pdb) format and the structure having rank 1 was used for further analysis. The predicted structures were further compared to the known structure of AtWRI1 using CHIMERA 1.14 software.

The ‘Match−>align’ command was used for structure alignment and the results were obtained as superimposed structures along with the root mean square deviation (RMSD) values of C-alpha atoms and the peptide backbones. Further, potential binding of the proteins with putative AW-box sequence was predicted using HDOCK online server.

Specifically, the N-terminal regions R58-R228 of PaWRI1 and K81-R246 of PaWRI2 having the AP2 domains were used as the receptor and the dsDNA AW-box sequence of PaBCCP2(SEQ ID NO: 115) promoter along with +2bp upstream and downstream (CTCGCCACCTACAAAGAC) was used as the ligand for docking.

The amino acids corresponding to the identical position as the residues previously identified in AtWRI1, which recognize the AW-box conserved sequences, were user-defined as potential active site binding residues. The docking structures were obtained along with the docking scores and confidence scores. The interaction showing the lowest docking score (˜highest binding energy) and highest confidence score were downloaded in .pdb format and visualized using CHIMERA software.

RNA Extraction and qrt-PCR Analysis

Approximately 50 mg of N. benthamiana leaves, agro-infiltrated with different constructs (P19 control, P19+PaWRI1, P19+PaWRI2, and P19+PaWRI1+PaWRI2), were collected and immediately frozen in liquid nitrogen. The process for agro-infiltration previously described herein was used. Total RNA was extracted from each sample using the RNeasy Plant Mini Kit (QIAGEN, Germany) according to the manufacturer's instructions. RNA concentrations were measured using a NanoDrop spectrophotometer, and samples were diluted with RNase-free water to a final concentration of 4 ng/μl. The RNA samples were stored at −20° C. in RNase-free tubes (QIAGEN, Germany). Quantitative real-time PCR (qRT-PCR) was performed using the qScript One-Step SYBR Green qRT-PCR Kit (QuantaBio) following the manufacturer's protocol and gene-specific primers of SEQ ID No: 14 to SEQ ID NO: 114.

20 ng of RNA was mixed in a 20 μL reaction mixture. All reactions were performed in an Illumina Eco Real-time PCR system with three biological replicates and two technical replicates. cDNA synthesis was performed at 50° C. for 10 minutes using gene-specific primers, followed by polymerase activation at 95° C. for 10 minutes. PCR cycles included denaturation for 10 seconds at 95° C., followed by annealing for 20 seconds at 60° C. for 20 seconds, repeated for 40 cycles. Fluorescence signals (Channel 1; for SYBR Green) were recorded after each cycle. The reference gene L23 was used as a control. Gene expression levels were quantified based on the quantification cycle (Cq) values determined from amplification curves using a threshold set at 0.02. Relative gene expression was then calculated as 2^{-ΔΔCq}.

### Yeast-One-Hybrid (Y1H) Assay

The Matchmaker Gold yeast one-hybrid kit (TaKaRa Bio, USA) was used for the assessment of the binding of PaWRI1 and PaWRI2 to the AW/AWL-box elements present in the promoters of the target genes.

### Competent Cell Preparation

The Y1HGold strain (MATα, ura3-52, his3-200, ade2-101, trp1-901, leu2-3, 112,gal4Δ, gal80Δ, met-, MEL1) of yeast (Saccharomyces cerevisiae) was streaked onto YPDA agar plates and grown at 30° C. for 3 days until colonies appear. One healthy colony (>2 mm diameter) was chosen and inoculated into 2 ml YPDA liquid media and grown overnight at 30° C. with shaking at 250 rpm. 20 μl of the saturated culture was further inoculated into 50 ml of YPDA liquid culture and grown at the same conditions until the OD₆₀₀ reached 0.3.

The culture was centrifuged at 700× g for 5 min, resuspended in 100 ml YPDA media and further grown for 3 hours until the OD₆₀₀ reached 0.4-0.5. The grown culture was centrifuged at 700× g for 5 min, resuspended with 30 ml sterile deionized water, followed by another round of centrifugation at 700× g for 5 min. The supernatant was discarded, and the pellet was resuspended with 1.5 ml of 1.1× TE/LiAc [1.1 ml 10× TE buffer+1.1 ml 1M Lithium acetate (LiAc)+7.8 ml deionized H₂O=10 ml 1.1× TE/LiAc] solution by vortexing and transferred to 2 ml microcentrifuge tubes. After centrifugation at 4,500× g for 15 s, the cells were finally resuspended with 600 μl of 1.1× TE/LiAc solution to prepare competent cells and stored at −80° C. until further use.

### Construction of Bait Plasmid

All the identified AW-box DNA sequences were used as bait and were tested for their ability to interact with PaWRI1 and PaWRI2. Although multiple AWL1 and AWL2-box sequences were identified in most of the analyzed promoters, only the ones closest to the Transcription Start Site (TSS) were used. Two base pairs upstream and downstream of AW/AWL-boxes were also included to give genomic context to each sequence. Furthermore, ‘GCTGGACC’ and ‘GCGTTAGC’ random base pairs were included in the 5′-upstream and 3′-downstream to increase the DNA annealing temperature. Finally, two antiparallel oligonucleotides were designed to have HindIII and SalI recognition site sticky end overhangs at 5′ and 3′-end, respectively. Both strands of the synthetic oligonucleotides were obtained from Eurofins Genomics and diluted with TE buffer to a final concentration of 100 μM. The top and bottom strand oligos were mixed at 1:1 (v/v) ratio and heated in thermal cycler at 95° C. for 30 s, 72° C. for 2 min, 37° C. for 2 min, 25° C. for 2 min, and finally chilled on ice to obtain dsDNA oligos with sticky end overhangs at a concentration of 50 μM. The pAbAi vector was linearized by restriction digestion with SalI-HF (NEB) and HindIII-HF (NEB) according to manufacturer's protocol and further purified using DNA clean & concentrator kit (Zymo Research). The dsDNA oligos were further cloned into the linearized vector using T4 DNA ligase (NEB) to generate pBait-AbAi plasmids.

The ligated plasmids were transformed into DH5α strain of E. coli and plated onto LB-Agar media containing Ampicillin (100 μg/ml) and grown overnight in 37° C. incubator. The positive transformant was confirmed by colony PCR using 5′-AAGCTTGCTGGACC-3′ forward and 5′-GTCGACGCTAACGC-3′ reverse primers. Following growing in LB+Amp liquid media for overnight in 37° C. shaker at 250 rpm, plasmid was isolated using GeneJet Plasmid miniprep kit (Invitrogen) and stored in −20° C. until further use.

### Generation of Bait Yeast Strain

The previously cloned pBait-AbAi plasmids along with the control p53-AbAi were linearized through restriction digestion by BstBI (NEB) according to manufacturer's protocol and column purified. The concentration of each linearized plasmid was measured by nanodrop and 200 ng of each plasmid was transformed into Y1HGold competent yeast. Specifically, the plasmid DNA, 5 μl of yeastmaker denatured carrier DNA (10 μg/ml), 50 μl yeast competent cells, and 500 μl PEG/LiAc solution (8 ml of 50% PEG 3350+1 ml 10× TE buffer+1 ml 1M LiAc=10 ml PEG/LiAc) were mixed in a prechilled microcentrifuge tube and incubated at 30° C. for 30 min. After adding 20 μl DMSO, the tubes were further kept in 42° C. water bath for 15 min. The cells were centrifuged at 4500× g for 15 s and the cell pellet was resuspended in 1 ml YPD plus medium. After incubation in 30° C. shaker at 250 rpm for 90 min, the cells were again centrifuged at 4500× g and resuspended in 500 μl of 0.9% (w/v) NaCl solution. 100 μl was plated onto SD-URA agar media and kept in 30° C. incubator for 3-5 days until colony appeared. The

Positive transformant was confirmed by colony PCR using the Matchmaker Insert Check PCR Mix 1 kit (TaKaRa) and competent cells were prepared as described before.

The background expression of the AureobasidinA resistant (AbA′) gene, cloned downstream of each bait, was tested by growing the positive transformants on media containing different concentrations of AbA. Specifically, the positive colony was picked and resuspended in 1 ml of 0.9% NaCl solution and further diluted to 10⁻¹, 10⁻², 10⁻³ concentrations. 5 μl of each cell solution was spotted onto SD-URA agar media plates containing different concentrations of AbA. The minimal inhibitory concentration for each pBait-AbAi yeast strain was found to be 200 ng/ml of AbA. The minimal inhibitory concentration for the control (p53-AbAi) was recommended at 100 ng/ml by the manufacturer.

### Assessment of Prey-Bait Interaction

Previously cloned putative PaWRI1 and PaWRI2 cDNA sequences were PCR amplified using specific primers to include Smal and Xhol restriction sites at the 5′ and 3′-end, respectively. The PCR products were digested with Smal and Xhol restriction enzymes and cloned into the pGADT7 AD destination vector at the corresponding restriction sites using T4 DNA ligase (NEB) according to the manufacturer's protocol to generate PaWRI1-GADT7 AD and PaWRI2-GADT7 AD prey plasmids. The ligated plasmids were transformed into T1^{R} one shot competent E. coli and plated onto LB agar plates containing Ampicillin antibiotic (100 μg/ml) and grown overnight in 37° C. incubator. Positive transformation was confirmed by colony PCR and further grown in LB+Amp liquid media and plasmid was extracted.

All the prey plasmids (PaWRI1-GADT7 AD, PaWRI2-GADT7, pGADT7) were linearized by restriction digestion with Smal and 250 ng was transformed into the bait Y1HGold competent yeast as previously described. For the p53 positive control, 125 ng of p53 DNA fragments were mixed with 1 μg of pGADT7-Rec plasmid (Smal linearized) and co-transformed into the p53-AbAi competent yeast. The transformed yeast cells were finally resuspended in 500 μl of 0.9% NaCl solution and further diluted to 10-1, 10-2, and 10-3 concentrations and 5 μl was spotted onto both SD-URA permissive and SD-LEU+AbA250 selective media (100 ng/ml of AbA used for p53 positive control) plates. All the media plates were kept in 30° C. incubator for 3 days and images were taken after that.

### Results and Discussion

Both PaWRI1 and PaWRI2 Induced Several FA and TAG Biosynthesis Genes in N. benthamiana Leaves

To identify which genes were transactivated by PaWRI1 and/or PaWRI2 the expression of various genes catalyzing the glycolysis, FA biosynthesis, and TAG assembly was quantified via qRT-PCR using the RNA extracted from N. benthamiana leaves.

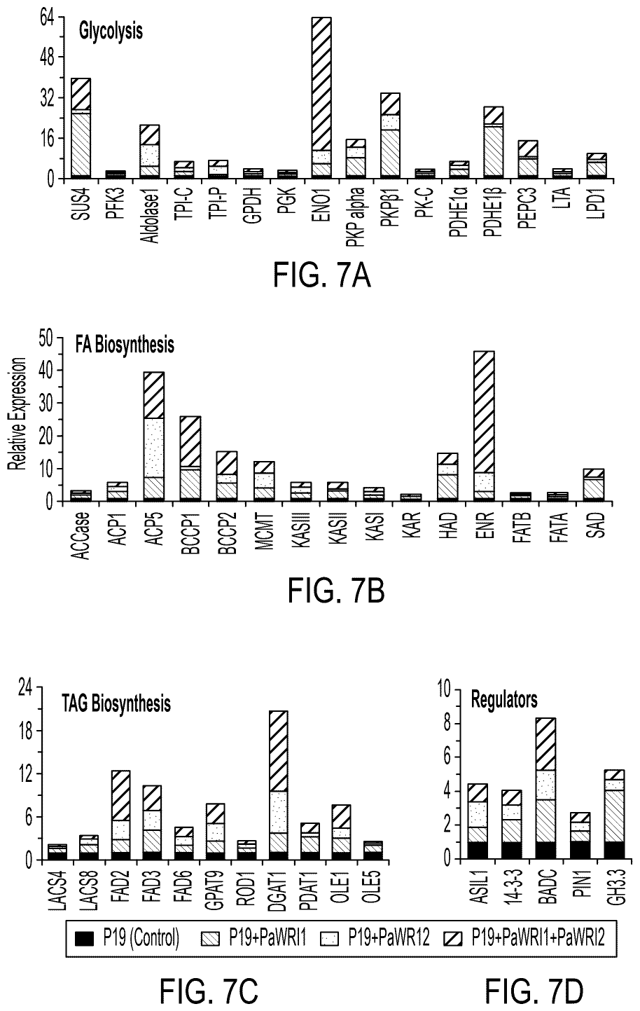

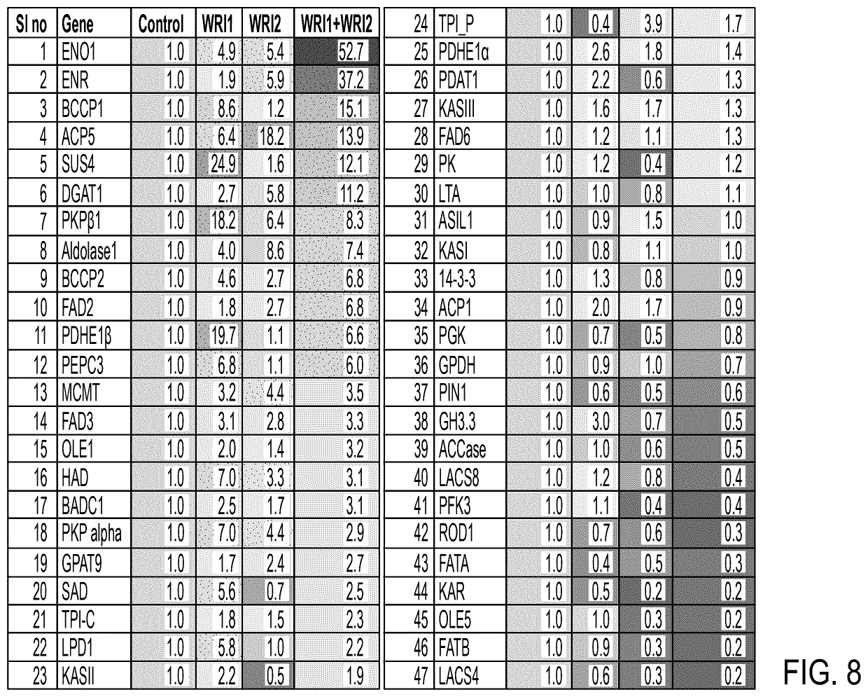

While most of the glycolytic enzymes were upregulated by PaWRI1 and/or PaWRI2, phosphofructokinase 3 (PFK3), glyceraldehyde-3-phosphate dehydrogenase (GPDH) (SEQ ID NO: 125), and phosphoglycerate kinase (PGK) (SEQ ID NO: 126) were downregulated, as shown in FIG. 7A and FIG. 8.

Among others, sucrose synthase 4 (SUS4), plastidial pyruvate kinase subunit alpha (PKpα) (SEQ. ID NO. 127), plastidial pyruvate kinase subunit beta 1 (PKp-β1) (SEQ ID NO: 120), pyruvate dehydrogenase E1 subunit a (PDHE1α) (SEQ ID NO: 121), phosphoenolpyruvate carboxylase 3 (PEPC3) (SEQ ID NO: 123), and lipoamide dehydrogenase 1 (LPD1) (SEQ ID NO: 124) subunit of pyruvate dehydrogenase complex are selectively upregulated by PaWRI1 (FIG. 7A and FIG. 8). Similarly, aldolase1, plastidial triose-phosphate isomerase (TPI-P), and enolase 1 (ENO1) are selectively upregulated by PaWRI2.

Co-expression of PaWRI1 and PaWRI2 upregulated SUS4 (SEQ ID No: 116), aldolase1, cytosolic TPI (TPI-C) (SEQ ID NO: 117), TPI-P (SEQ ID NO: 118), ENO1 (SEQ ID NO: 119), PKp-β1, PDHE1α, PDHE1β (SEQ ID NO: 122), PEPC3, and LPD1. Surprisingly, co-expression of PaWRI1 and PaWRI2 upregulated expression of PKpα and PDHE1α, but the effect is less compared to that shown by either PaWRI1 and PaWRI2 alone.

The most notable effect was observed in the ENO1 expression. While PaWRI1 and PaWRI2 increased ENO1 expression by ˜4.8 and ˜5.3-fold, respectively, co-expression synergistically increased ENO1 expression by ˜52.6-fold.

The increased expression of SUS4 in N. benthamiana leaves, induced by co-expression of PaWRI1 and PaWRI2, suggests a metabolic shift towards enhanced fatty acid (FA) biosynthesis by increasing hexose content through sugar catabolismas the SUS enzyme catalyzes the cleavage of sucrose into fructose and UDP-glucose which may be fed into various metabolic pathways, including glycolysis. These results, including a reduction in PFK3 expression, indicating a preference for plastidial glycolysis over cytosolic glycolysis, as PFK3 is localized in the cytosol. This shift is further supported by decreased expression of cytosolic GPDH, PGK, and PK-C genes, alongside increased expression of plastidial aldolase1.

PaWRI2 alone and the co-expression samples showed higher plastidial TPI expression, whereas PaWRI1 alone did not. Elevated expression of plastidial ENO1, PKp-α, and PKp-β1 subunits of pyruvate kinase suggests that hexose sugars predominantly enter plastidial glycolysis. Moreover, the synergistic increase in the plastid localized ENO1 expression further indicates that the hexose sugars mostly enter plastidial glycolysis

These results demonstrate that increased FA biosynthesis in N. benthamiana leaves is driven by enhanced plastidial glycolysis and fatty acid biosynthesis gene expression and demonstrates the metabolic coordination facilitated by the co-expression of PaWRI1 and PaWRI2, thereby promoting lipid accumulation in plant cells.

The expression of genes involved in de novo fatty acid (FA) biosynthesis was also evaluated. Consistent with FA biosynthesis occurring in the plastid, we observed a decrease in cytosolic acetyl-CoA-carboxylase (ACCase) expression. Furthermore, the plastidial ACCase subunits, biotin carboxyl carrier protein 1 and 2 (BCCP1 and BCCP2), showed elevated expression levels in N. benthamiana leaves expressing PaWRI1 and/or PaWRI2. While PaWRI1 appeared to strongly induce both genes, co-expression of PaWRI1 and PaWRI2 led to additive improvements in their expression.

Of the two acyl carrier proteins (ACP1 and ACP5), both PaWRI1 and PaWRI2 individually increased their expression. However, co-expression of PaWRI1 and PaWRI2 in N. benthamiana leaves resulted in a decrease in ACP1 expression and an increase in ACP5 expression. Additionally, the gene encoding malonyl-CoA: ACP transacylase (MCMT), responsible for transferring the malonyl group from malonyl-CoA to ACP was upregulated in all over-expressing leaf samples.

Increased expression of hydroxyacyl-ACP dehydratase (HAD) (SEQ ID NO: 128) and enoyl-ACP reductase (ENR) (SEQ ID NO: 129), which catalyze the successive dehydration and reduction reactions to form saturated acyl-ACP, was observed. Notably, ENR exhibited a synergistic increase in expression when PaWRI1 and PaWRI2 were co-expressed. Furthermore, a reduced expression of genes encoding acyl-ACP thioesterases FATA (SEQ ID NO: 130) and FATB (SEQ ID NO: 131), which determine the amount and type of fatty acids exported from the plastid to other cellular compartments, was observed.

Without being bound by theory, since green tissues like leaves typically do not overproduce storage oils, the reduced expression of FATA and FATB may serve as a metabolic checkpoint to prevent excessive oil accumulation in leaves, despite the high expression levels of other fatty acid biosynthesis genes in N. benthamiana. Additionally, the expression of plastidial stearoyl-ACP desaturase (SAD) (SEQ ID NO: 132) was upregulated in PaWR1 leaves and the co-expression leaves. Plastid-localized SAD catalyzes the desaturation of C18:0-ACP to C18:1-ACP.

Overall, the reduced expression of FATA and FATB, combined with increased SAD expression, suggests an elevated oleic acid (C18:1) and linolenic acid (C18:3) content at the expense of reduced C16:0 and C18:0 levels.

Next, the expression of fatty acid (FA) modification genes and triacylglycerol (TAG) assembly genes in N. benthamiana leaves was analyzed. Among the two ER-localized long-chain acyl-CoA synthetases (LACS4 (SEQ ID NO: 133) and LACS8 (SEQ ID NO: 134)) studied, neither showed upregulated expression. Instead, their expression decreased in leaf tissue when PaWRI1 and PaWRI2 were expressed independently or together.

In avocado mesocarp, LACS4 is the most abundantly expressed LACS gene, followed by LACS8, LACS1, and LACS9. This contrasts with observations in oil-rich seed tissues and non-seed tissues, where LACS9 is among the major and abundantly expressed genes. In avocado mesocarp, the ER-associated isoforms (LACS1, LACS4, and LACS8) represent about 80% of LACS transcripts, while LACS9 represents about 16%. This suggests that FA export from plastid to ER in avocado mesocarp might follow a different mechanism.

Among the fatty acid desaturase (FAD) paralogs analyzed in leaf tissue, elevated expression was observed for ER-localized FAD2 (SEQ ID NO: 135) and FAD3, while plastidial FAD6 (SEQ ID NO: 136) showed little upregulation. Desaturation of C18:1 to C18:2 is catalyzed by FAD2 and FAD6, and further desaturation to C18:3 is catalyzed by FAD3. Previous observations noted increased expression of plastidial SAD induced by coexpression. These findings suggest that the initial desaturation of C18:0 to C18:1 occurs in the plastid, with intermediates then transported to the ER for further desaturation, leading to PUFA production. Consequently, the higher C18:3 content in PaWRI1 and PaWRI2 co-expression samples, compared to single overexpression, can be attributed to increased activity of FAD2 and FAD3.

Increased expressions of TAG assembly genes GPAT9 (SEQ ID NO: 137), DGAT1, and PDAT1 were observed in PaWRI1 and co-expressing leaves, whereas PDAT1 expression was reduced in PaWRI2 samples. Among the acyltransferases, DGAT1 was more prominently upregulated by PaWRI2 and co-expression. Typically, TAG assembly genes are not transactivated by WRI1, except for ROD1 (SEQ ID NO: 138). However, reduced expression of ROD1 was noted in all test leaf samples. The ROD1 gene encodes the phosphatidylcholine cholinephosphotransferase (PDCT) enzyme, which interconverts DAG and phosphatidylcholine (PC) molecules. Reduced ROD1 expression in N. benthamiana leaves suggests that most substrates for TAG assembly are derived via the Kennedy pathway, supported by the very high expression levels of DGAT1 compared to PDAT1.

Elevated expression of the lipid droplet-associated protein Oleosin 1 (OLE1) (SEQ ID NO: 139), but not OLE5, was also observed. Since OLE genes are not usually transactivated by WRI homologs, the increase in OLE1 might be due to an increase in the metabolic flux for improved TAG biosynthesis. The increased expression of desaturase and TAG assembly genes suggests that the metabolic flux of FA substrates into the ER created positive feedback, inducing the expression of associated genes. The activities of these native enzymes in the leaves likely contributed to the altered FA composition observed in the co-expression study (FIGS. 7A-7D and FIG. 8)

Furthermore, the expression of genes encoding proteins that regulate WRI1 stability and expression was analyzed. The trihelix transcription factor ASIL1 (SEQ ID NO: 140), known to repress WRI1, showed unaltered expression in N. benthamiana leaves expressing PaWRI1 and co-expressing PaWRI1 and PaWRI2. However, PaWRI2 induced a small increase in ASIL1 expression, suggesting an alternate role in regulating WRI2 expression. The phosphopeptide binding protein 14-3-3, which interacts with and stabilizes WRI1, was largely unaffected in all leaf samples. In contrast, the gene encoding biotin attachment domain containing 1 (BADC1) (SEQ ID NO: 141), which senses increased FA synthesis and inhibits ACCase activity, exhibited increased expression triggered by PaWRI1 and/or PaWRI2.

In Arabidopsis, WRI1 directly transactivates BADC paralogs through binding to the AW-box present in their promoters to conditionally repress FA biosynthesis. Therefore, the increased BADC1 expression might serve as a regulatory mechanism to counterbalance and offset the excessive production of FA in the leaves. Additionally, the expression of two auxin metabolism genes, PIN-formed (PIN) (SEQ ID NO: 142) and GH3.3 (SEQ ID NO: 143), previously shown to be transactivated by WRI1, was analyzed. PIN functions in auxin transport, while GH3.3 is responsible for its degradation.

Decreased expression of these two genes was observed in all samples, except for an upregulation of GH3.3 in leaves expressing PaWRI1. While AtGH3.3 has a canonical AW-box in its promoter, AtPIN genes have a non-canonical AW-box (AW-box-like), to which AtWRI1 binds. Therefore, it is possible that only PaWRI1 binds to the AW-box in NbGH3.3 and thus the WRI-mediated auxin signaling may be exclusively controlled by WRI1, not WRI2.

### AW and AW-Like Box Elements are Present in the Promoter of Numerous Avocado Lipid Metabolism Related Genes

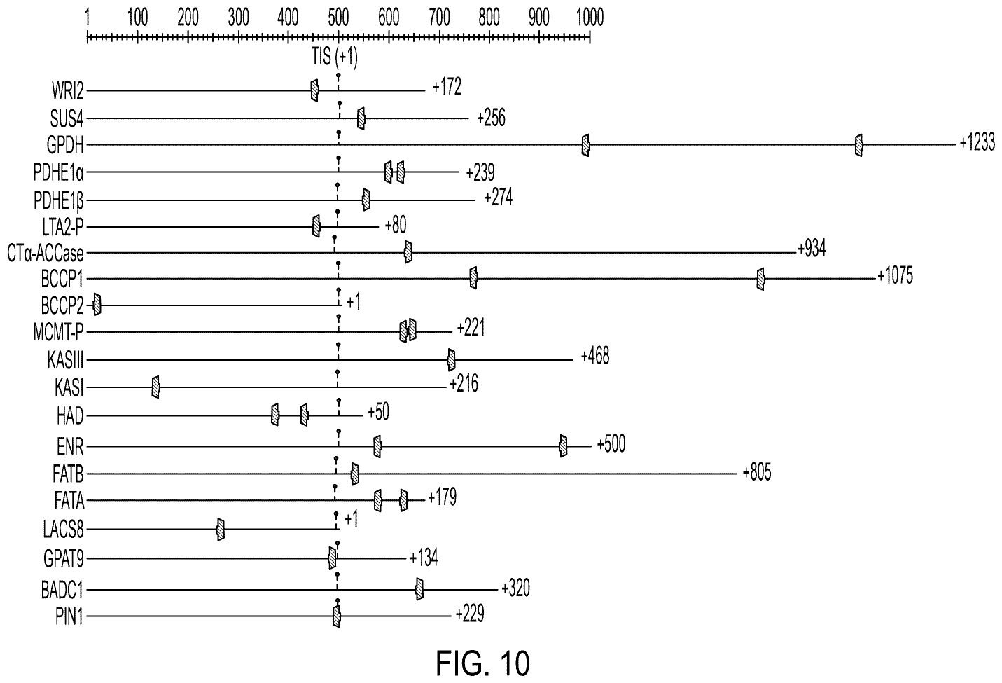

Both PaWRI1 and PaWRI2 induced the expression of FA-biosynthesis and TAG assembly genes in N. benthamiana leaves, prompting further investigation into their regulatory roles in the native avocado system. A search for canonical AW-box [CnTn(n)7CG] as well as non-canonical AWL1 [CG(n)6CnAnG] and AWL2 [G(n)7CnAnG] sequences in the promoter regions of the 47 genes previously analyzed by qRT-PCR was conducted. The focus was on the 500 bp upstream (−500) sequence of the transcription start site (TSS: +1) and the untranslated region (UTR) up to the start codon (ATG) for AW/AWL-sequence search. As shown in FIG. 10, among the genes, 20 contained AW-boxes in their promoter regions, with most genes having one AW-box, except for GPDH, PDHE1α, BCCP1, MCMT, HAD, and FATA, which had two. While most AW-boxes were located in the UTR region and close to the TSS, BCCP2 and KASI were exceptions. AWL-boxes were also identified in several gene promoters, distributed both upstream of the TSS and in the UTR regions. Further analysis revealed that AW-boxes in Brassicaceae genomes are more likely to interact with WRI1 and transactivate the gene if they are present within ±250 bp of the TSS, although exceptions exist. To investigate these interactions, all the AW-box sequences and only the AWL-sequences closest to the TSS were selected for further DNA-protein interaction studies using the Y1H assay.

### Both PaWRI1 and PaWRI2 Have an Intact AP2 Domain

To identify the sequence-specific features of PaWRI1 and PaWRI2, multiple sequence alignments were conducted with their orthologs in Arabidopsis (data not shown). The AP2 domain sequences were the primary focus, as the residues responsible for AW-box recognition were recently discovered. Previous studies have shown that WRI2-orthologs evolved independently and earlier than their corresponding paralogs in the respective plant species and possess only one AP2 domain, unlike other paralogs that have two. Over 45% of shared identity is found in the N-terminal AP2 domains, a characteristic of the APETALA protein family. The N-terminal region of WRI1-orthologs in plants is highly conserved, while the C-terminal region containing the TAD is variable.

Among the two AP2 domains, the first domain is typically more conserved than the second, a trend also observed in this sequence comparison. However, the single AP2 domain in AtWRI2 is interrupted by a stretch of 35 additional amino acids (Behera et al., Functional and Predictive Structural Characterization of WRINKLED2, A Unique Oil Biosynthesis Regulator in Avocado, FRONT PLANT 12:992 SCI, (2021), available at www.frontiersin.org/article/10.3389/fpls.2021.648494). Despite having only a single AP2 domain, plant AINTEGUMENTA transcription factors can bind DNA promoters. However, Y1H studies have shown that AtWRI2 is unable to bind to the promoter of the BCCP2 gene, likely due to the interrupted AP2 domain. This suggests that an intact AP2 domain is required for binding DNA promoter motifs, explaining the lack of function for AtWRI2.

The recently elucidated 3-D structure of AtWRI1 showed that the residues H72, E79, W83, and Q98 in the first AP2 domain recognize the ‘CnTnG’ sequence of the AW-box, while H175, E181, and R183 are responsible for recognizing the conserved ‘CG’ sequence. All four residues in the first AP2 domain are conserved across the sequences. However, only PaWRI1 shows conservation of all three residues in the second AP2 domain at similar positions. In contrast, PaWRI2 shows conservation of ‘E’ at position 199, corresponding to E181 in the second AP2 domain of AtWRI1. Without being bound by theory, given that PaWRI2 can induce FA-biosynthesis genes, PaWRI2 may recognize only the ‘CnTnG’ sequence. Alternatively, it might form a dimer to provide two AP2 domains for canonical AW-box recognition and binding.

### Both PaWRI1 and PaWRI2 can Recognize AW-Box

The structures of PaWRI1 and PaWRI2 were predicted using AlphaFold2. The results indicate that substantial regions of these proteins are disordered, except for the regions spanning the AP2 domains (data not shown). Both PaWRI1 and PaWRI2 have B-sheets followed by an α-helix in their predicted AP2 domains.

Structural comparison with AtWRI1 was conducted, focusing only on the region spanning the AP2 domains (57-227 for PaWRI1 and 80-246 for PaWRI2). Comparison of key residues responsible for AW-box recognition revealed that, although the residues are conserved in PaWRI1, structural deviations were observed. Similar deviations were observed in the single AP2 domain of PaWRI2. Without being bound by theory, PaWRI1 and PaWRI2 may bind to the AW-box recognition sequence with different affinities.

DNA binding strength analysis was conducted using PaWRI1 and PaWRI2 as receptors and the AW-box dsDNA sequence in the avocado ProBCCP2 as the ligand. Both PaWRI1 and PaWRI2 showed docking scores below −200, suggesting that both are likely to bind to the AW-box. PaWRI2 showed a lower docking score, indicating a higher binding strength.

### PaWRI2 is Both Self-Transactivated and Activated by PaWRI1

An AW-box recognition sequence was identified in the promoter region of PaWRI2, leading to the investigation of whether it is transactivated by avocado WRI1 and WRI2. A Y1H assay was conducted to assess the ability of these WRI paralogs to bind to the AW-box. The detailed workflow of the Y1H assay is described in the methods section. Given that Y1H assays can produce false positives due to leaky expression from native yeast promoters, the minimum inhibitory concentration of the selective antibiotic Aureobasidin A (AbA) was optimized and determined to be effective at 200 ng/ml. Therefore, 250 ng/ml was used in all Y1H assays, except for the positive control p53, which used 100 ng/ml as recommended by the manufacturer.

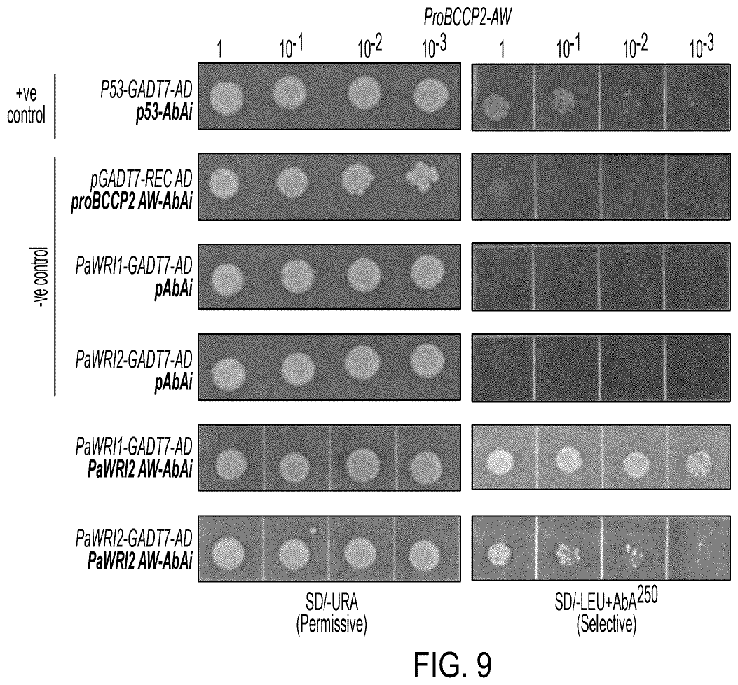

Initial assessments using positive and negative controls produced expected results. Further assessment of PaWRI1 and PaWRI2 binding to the proPaWRI2 AW-box revealed that both proteins could bind and transactivate the gene, with PaWRI1 exhibiting stronger binding compared to PaWRI2 (see FIG. 9).

Transcriptome analysis of expressed genes in avocado mesocarp showed higher expression of PaWRI2, comparable to PaWRI1, which further increased in stage 4 of fruit growth. This suggests that both PaWRI1 and PaWRI2 might increase the expression of PaWRI2 in later stages of fruit development through transactivation. This indicates a novel interdependent regulatory network of WRI paralogs in non-seed tissues, such as avocado mesocarp, which has not been documented previously. No similar regulatory relationship among seed tissue WRI paralogs is known, as WRI1 is the major candidate responsible for seed oil biosynthesis. Although WRI1 has been shown to negatively autoregulate its expression, it does not directly bind to its own promoter. Therefore, the ability of both PaWRI1 and PaWRI2 to bind to the AW-box promoter element of PaWRI2 suggests they are likely to transactivate the gene. These findings reveal a new trans-regulatory network in non-seed tissue.

Both PaWRI1 and PaWRI2 were Able to Regulate FA-Biosynthesis Genes Through Binding to AW-Box

To test the interaction of PaWRI1 and PaWRI2 with lipid gene promoters, a Y1H assay was conducted using 84 AW-box and AW-box-like promoter elements from 47 avocado genes. The data suggested a complex, interdependent metabolic regulation by PaWRI1 and PaWRI2, both of which could bind to several AW-box and AWL-box promoter elements. PaWRI1 showed higher binding strength compared to PaWRI2, contrary to in silico predictions. Although both PaWRI1 and PaWRI2 have overlapping target gene AW-boxes, some differences were observed. All glycolysis gene AW-boxes could bind to both proteins, but GPDH AW2 showed no interaction with PaWRI1, indicating that GPDH AW1, closer to the translation initiation site (ATG), is preferred by PaWRI1.

Among the FA biosynthesis genes, the CTα subunit of ACCase (SEQ ID NO: 145) was induced by PaWRI2, whereas FATB was induced by PaWRI1 only. Neither protein could bind to the KASIII AW-box. For genes with two AW-box elements within their promoters (BCCP1, HAD, ENR, and FATA), both proteins could induce expression, except for ENR AW2. Overall, analysis of FA-biosynthesis genes showed that both PaWRI1 and PaWRI2 could induce their expression; PaWRI2 possibly increases de novo FA biosynthesis by increasing the expression of CTα, whereas PaWRI1 helps overcome the fatty acyl-CoA transport checkpoint by inducing FATB.

Among the ER-specific genes, both LACS8 and GPAT9 showed interaction of their AW-box with PaWRI1 and PaWRI2. Both PaWRI1 and PaWRI2 could induce the conditional repressor BADC AW-box, suggesting tight control of FA metabolism in avocado mesocarp. The interaction of the auxin metabolism gene PIN1 AW-box with neither protein suggests that non-conserved bases in the AW-box might specify the binding of WRI-paralogs to the promoter element, similar to observations in sunflower lipid genes.

Assessment of AWL1-box binding to avocado WRI-paralogs showed that PaWRI2 had almost no preference for them, whereas PaWRI1 could bind to a limited number of gene promoters, specifically PFK6 (SEQ ID NO: 144), Aldolase, GPDH, CTα, LACS8, and FAD2. The WRI4 AWL1-box showed binding with PaWRI1. Since avocado mesocarp did not show any expression of PaWRI4, it is possible that PaWRI1 represses its expression, requiring further validation. AWL2-boxes also showed selective preference for PaWRI1. However, not all promoter elements were induced by PaWRI1. Only SUS3, PDHE1β, CTα, HAD, DGAT1, and OLE1 gene promoters were induced by PaWRI1, suggesting that despite multiple potential binding sites, the avocado genes are not overly transactivated by WRI1 to avoid metabolic imbalance. Surprisingly, the avocado WRI1 AWL2 promoter element showed a binding preference for PaWRI1. Previous studies indicated that WRI1 is negatively autoregulated, but not through binding to the AW-box, suggesting a possible mechanism for this autoregulation.

### Conclusion

This example provides valuable insights into the regulatory roles of PaWRI1 and PaWRI2 in fatty acid (FA) biosynthesis and triacylglycerol (TAG) assembly in Nicotiana benthamiana leaves and avocado mesocarp. Numerous genes involved in glycolysis, FA biosynthesis, and TAG assembly were co-induced by PaWRI1 and PaWRI2, while several other genes were independently regulated by either transcription factor (TF). PaWRI1 and PaWRI2 exhibited different affinities for certain target genes, and their coexpression had a notable synergistic effect, particularly increasing the expression of key genes ENO1 and ENR in N. benthamiana leaves. Both genes selectively transactivate plastidial glycolytic enzymes while suppressing the expression of cytosolic enzymes.

Although FA genes were largely upregulated, the low expression of FATA and FATB in leaves represents a bottleneck for further storage oil biosynthesis. In silico analysis showed that both PaWRI1 and PaWRI2 can potentially bind to AW-box and AWL-box promoter elements of various genes related to lipid metabolism in avocado, which was further confirmed by Y1H assay. Avocado WRI2 is transactivated by itself and by PaWRI1, representing a novel, complex, and interdependent regulatory network of these transcription factors in avocado fruit development and lipid metabolism.

Further Y1H assays established that both PaWRI1 and PaWRI2 are capable of transactivating lipid biosynthesis genes with a preferential and selective binding to the AW-box in their upstream proximal promoters. These findings shed light on the molecular mechanisms underlying lipid biosynthesis.

### Example 3: Identification of Transactivation Domain of Oil-Biosynthesis Regulator Proteins WRI1 and WRI2 in Avocado Through Domain Deletion

Plant proteins, especially transcription factors (TFs), often lack a fixed three-dimensional structure due to amino acid composition bias, resulting in intrinsically disordered regions (IDRs) critical for function. WRINKLED1 (WRI1), the master regulator of oil biosynthesis in plants, exhibits a highly disordered structure in Arabidopsis with three IDRs (IDR1,2, and 3). IDR3 in AtWRI1 contains a ‘PEST’ motif recognized by degrading enzymes and overlaps with the C-terminal transactivation domain (TAD).

In avocado (Persea americana), an unusual co-expression of PaWRI1 and PaWRI2 paralogs in oleaginous mesocarp tissue contributes to oil biosynthesis, as demonstrated by transient expression in N. benthamiana leaves. In silico analyses showed that PaWRI2 lacks a C-terminal ‘PEST’ motif and IDR3, while PaWRI1, though possessing IDR3, displays an ordered region in the C-terminus.

To identify the TAD in both paralogs, C-terminal deletion constructs were strategically engineered and transiently expressed in N. benthamiana leaves. The studies revealed an improvement in transactivation capabilities, leading to increased lipid and fatty acid content in N. benthamiana leaves when the ordered fragment at the C-terminus was removed. The results suggest that the IDR2 domain in PaWRI2 constitutes the TAD. Additionally, an increase in the proportion of C18:0 and C18:1 was observed when the C-terminal ordered region truncated constructs (PaWRI1³⁷⁵ and PaWRI2³⁸¹) were expressed in leaf tissue.

Overall, this example suggests that the C-terminal region of WRI paralogs can be manipulated to improve their function.

### Materials and Methods

### In Silico Analyses

The prediction of IDRs was conducted using the Predictors of Natural Disordered Regions (PONDR-VL3) online tool. The disordered regions in the full-length protein sequences were identified and provided as graphic format outputs. A score above the threshold value of 0.5 indicates a propensity of that stretch of protein to be a part of an IDR. Only the stretches of >30 amino acid residues showing disordered tendency are considered as likely IDRs in the protein tertiary structure.

### Cloning of C-terminal Deletion Constructs

The DNA fragments encoding for the C-terminal deletion constructs of PaWRI1 and PaWRI2 were PCR amplified using previously cloned PaWRI1-pK34 and PaWRI2-pK34 vectors. Following digestion with NcoI and SacII restriction enzymes, the amplicons were cloned into the pK34 entry vector between CaMV35S promoter and terminator using T4 ligase enzyme (NEB) according to the manufacturer's protocol. 5 μl of the ligated products were transformed into Top10 E. coli competent cells and were grown overnight at 37° C. on LB agar media plates containing ampicillin (100 μg/ml). Positive transformants were confirmed by colony PCR and plasmids were extracted. Following digestion by AscI, the generated promoter: gene: terminator cassettes were cloned into the corresponding restriction site in the pB110 binary vector and transformed into E coli following similar transformation method. After growing overnight in LB agar containing kanamycin (50 μg/ml), the positive transformants were confirmed by colony PCR. The viral silencing suppressor protein coding gene (P19 ) previously cloned into pB110 vector was used as control.

Agroinfiltration into Nicotiana benthamiana Leaves

All the cloned gene constructs, along with the P19 control, were co-infiltrated into the N. benthamiana leaves through Agrobacterium-mediated transformation. Specifically, 100 μL of Agrobacterium LBA4404 competent cells were gently mixed with 100 ng of the binary plasmid vectors expressing the genes of interest and cooled on ice for 5 min followed by freezing in liquid nitrogen. The frozen cells were then kept in 37° C. water bath for 5 min and subsequently mixed with 1 ml of LB liquid media. Following growth in 28° C. shaker for 2-4 hours, the cells were pelleted by centrifugation at 400× g and further resuspended with 100 μL LB liquid media and then plated onto LB agar plates containing kanamycin (50 μg/ml) and rifampicin (50 μg/ml) antibiotics. The plates were kept in 28° C. incubator for 3 days and positive colonies were retrieved. One of the positive colonies was inoculated into LB media containing appropriate antibiotics and grown overnight in a shaker (250 rpm) and 50 μl was further sub-cultured into 5 ml of LB media until the OD600 reached ˜0.6-0.8. The cells were centrifuged at 4000× g for 5 min at room temperature and the pellet was resuspended in appropriate volume of infiltration buffer (5 mM MgSO4, pH 5.7, 5 mM methyl ethanesulfonate, and 100 mM acetosyringone) to achieve a final OD600 of 0.3. After keeping in darkness at room temperature for 2 hours, the cells were ready for infiltration.

Wild type N. benthamiana plants were used for transient expression of all the genes. The plants were grown in a growth chamber (Percival Scientific, Perry, IA, USA) under long day conditions (16 h L/8 h D) at 24° C. and 60% relative humidity. Leaves of similar size (5-7 cm diameter) from six weeks old plants were used for transient expression. The Agrobacterium infiltration solution harboring the genes and the P19 were mixed with equal volume ratio and the P19 alone was used as control in each experiment. Two leaves in each plant and three such plants (n=6) were used for agro-infiltration. All the samples for comparison were randomly infiltrated into different areas in each leaf and the plants were further kept in growth chamber for 6 days in ambient growth conditions to facilitate gene expression. Leaf samples from the infiltrated areas were harvested and stored at −80° C. until further use.

### Nile Red Staining and Confocal Microscopy to Visualize Lipid Droplets (LDs)

A small portion of the harvested leaf sample was cut out using leaf puncher and used for staining. Nile Red (in dimethyl sulfoxide) was diluted with PIPES buffer (50 mM, pH 7.0) to a final concentration of 2 μg/ml and used for staining of LDs in the leaves. After 20 min of staining, the samples were washed three times (15 min each) with PIPES buffer and leaf discs were immediately observed using a Leica TCS SP8 confocal fluorescence microscope. The excitation wavelength was 488 nm and the emission wavelengths were 560-620 and 640-720 for detection of fluorescence emitted by Nile Red-stained LDs and chlorophyll autofluorescence, respectively. All the images were taken at 40× magnification and 45 z-stacks from 30 μm depth were superimposed to create the final image. The number of LDs were quantified using imageJ software. Three panels from each biological replicate (104 μm2), for a total of nine panels, were analyzed to quantify the LDs. Data were represented as mean±SD.

### Lipid Extraction and Analysis

The leaf samples collected 6 days post-infiltration were used for lipid extraction. 50 mg of plant tissue (fresh weight, FW) was homogenized using bead beater (BioSpec Inc., USA) after adding 2 ml isopropanol and then heated at 70° C. for 30 min to inactivate any internal lipase activity. Before homogenization, 50 μg of glyceryl triheptadecanoic acid (tri-17:0) standard (Sigma-Aldrich) was added to the plant tissue. After cooling to room temperature, 1 mL of chloroform and 400 μL water were added to achieve a final ratio of 2 mL isopropanol: 1 mL chloroform: 0.45 ml water. The samples were stored overnight at 4° C. The next day, the supernatant was collected from the sample after vortexing and centrifugation for 5 min at 4000×g. One mL of chloroform and 2 mL of 1M KCl were added to the sample to achieve phase separation. After centrifugation at 1500×g, the upper aqueous phase and interphase were aspirated by using a Pasteur pipette. The aspiration was repeated thrice with 2 mL of 1M KCl and the bottom organic phase was collected and dried under nitrogen gas. Dried lipid samples were reconstituted in 1 mL of chloroform and transferred into a pre-weighed glass vial. After evaporating the chloroform in nitrogen gas, each glass vial was weighed again to obtain the total lipid weights.

The dried lipid samples were dissolved in 100 μl hexane and 50 μl of them were used for fatty acid profile analysis by GC-FID (GC-2010 SHIMADZU). Fatty acid methyl ester (FAME) derivatives of the fatty acyl species present in the total lipid was prepared by adding 2 ml of methanolic HCl solution (1 M) to each sample followed by incubation in 85° C. water bath for 2 hours. After cooling to room temperature, 2 ml hexane and 2 ml KCl (1 M) was added to each sample and vortexed vigorously. The samples were then centrifuged at 4000×g for 10 min to facilitate phase separation and the upper hexane phase containing all the FAMEs were transferred to new vials. After drying in nitrogen vapor, 250 μl of hexane was added to each tube and transferred to the injector vials for gas chromatography. 1 μL of each sample was injected into the column for separation and detection by GC-FID. A capillary column (ZB-FAME; 30 m×0.25 mm I.D., 0.20 μm) with helium as carrier gas (flow rate of 4.6 mL/minute) was used. The injection temperature was set at 160° C., which was ramped after three minutes to 210° C. at 10° C./minute; the detection temperature was 255° C. The retention time for each fatty acid was determined by comparison to the spiked components of a standard FAME mix GLC 63C (Nu-check Prep Inc., USA). Fatty acids were quantified using a previously described method (Vanhercke) relative to the known quantity of tri-17:0 internal standard added to each sample prior to lipid extraction. 4-6 samples for each test group were used.

### Statistical Analysis

The transient expression data were expressed as their mean value with standard deviation (mean±SD). One-way analysis of variance (ANOVA) followed by Tukey's post-test was performed using Minitab software (version 18) at p-level of 0.05 (p<0.05) to test for significance among the data set.

### Results and Discussion

### Avocado WRI2 Lacks the C-terminal IDR3 Region

Intrinsically disordered regions (IDRs) in protein structures can prevent the formation of a hydrophobic core and result in improper folding. In all three WRI homologs, local random coil structures and amino acid biases contribute to IDRs. The propensity for disorderliness is about two-fold higher among WRI homologs analyzed compared to the mean value of 23% observed in the general Arabidopsis proteome. The overall disorder percentage among WRI1 orthologs is as follows: ZmWRI1a>AtWRI1>PaWRI1. For WRI2 orthologs, the order is PaWRI2>ZmWRI2>AtWRI2. While higher disorderliness promotes flexibility and protein interaction, it also increases accessibility to proteases and the likelihood of protein degradation. The relatively higher disordered values associated with both PaWRI2 and PaWRI3 and lower values with PaWRI1 suggest a complex homeostasis, where one protein compensates for the degradation of the other to achieve sustained fatty acid biosynthesis during the prolonged period of TAG accumulation in avocado mesocarp.

The percentage of disorder observed is reflected in the number of IDRs identified in each homolog, shown in the table below.

Details of the disorder value and the position of various intrinsically
disorder regions (IDRs) and PEST motifs in WRI homologs.

| Proteins | % Disorder | IDR1 | IDR1a | IDR2 | IDR3 | PEST motifs |
|---|---|---|---|---|---|---|
| PaWRI1 | 51.4 | 1-67 | * | 226-273 | 314-375 | 1-35, 228-247 |
| AtWRI1 | 52.5 | 1-71 | * | 249-313 | 368-430 | 3-34, 396-430 |
| ZmWRI1a | 56.7 | 1-67 | 135-175 | 238-313 | 356-395 | 6-32, 232-273, 273-319 |
| PaWRI2 | 54.2 | 1-85 | 171-201 | 266-381 | * | * |
| AtWRI2 | 45.4 | 1-57 | * | 278-377 | 426-458 | * |
| ZmWRI2 | 48.0 | 1-74 | * | 247-319 | 362-412 | * |

The protein sequences of PaWRI1, AtWRI1, and ZmWRI1 contain three, three, and four IDRs, respectively, while all WRI2 and WRI3 proteins show the presence of three IDRs. For further comparisons, the identified IDRs were named based on their position, similar to previously identified IDRs in AtWRI1: IDR1, IDR2, and IDR3. While IDR1 and IDR2 are unanimously predicted in all homologs, avocado WRI2 lacks the IDR3 in its C-terminal region. An additional short IDR (30-40 amino acids), referred to as IDR1a, is predicted in the linker region connecting the two AP2 domains (ZmWRI1a) and following the single AP2 domain in PaWRI2 proteins. The size of the IDRs varies among the homologs. Long IDRs (>30 amino acids) are common, comprising about 33% of the eukaryotic proteome, and affect transcription and translational activity, post-translational modifications, transportation and signal transduction activity, and also stability and degradation. Shorter IDRs in the linker regions typically offer conformation flexibility needed for DNA-binding proteins. The shorter IDR1a in the linker regions of some WRI orthologs and the other longer IDRs around the AP2 domains are likely to provide conformational flexibility for effective interaction with the target promoters, while IDRs in the C-terminus might be associated with protein stability.

Disordered N- and C-terminal regions are common in DNA-binding proteins, with the C-terminal IDR especially prevalent in transcription factors. Previously characterized AtWRI1 IDR3 has a functional role in transactivation, although a three-fold increase in expression upon the truncation of IRD3 suggests that its presence likely also serves as a target for protein degradation pathways. IDR3 is absent in PaWRI2, although the other analyzed WRI2 orthologs have an IDR in their C-terminus. However, PaWRI2 contains relatively long IDR1 and IDR2 (85 and 116 residues) compared to the respective orthologs in Arabidopsis and maize (57 and 100 residues, and 74 and 73 residues, respectively). These IDRs likely provide the necessary degree of transactivation capacity, a function lost in AtWRI2. The functional role of ZmWRI2 remains to be examined. Similarly, IDR3 is absent in yellow nutsedge (Cyperus esculentus) WRI1 (CeWRI1), although it retains its functional role in oil biosynthesis and shows an autoregulatory effect on the AtWRI1 promoter. Therefore, PaWRI2 may still activate target genes in a manner similar to the transactivation mechanism shown by CeWRI1. Long IDRs are also preferential targets for post-translational modification and protein binding, protecting them from proteolytic cleavage. The stability of AtWRI1 is enhanced upon interaction with either BTB/POZMATH1 (BPM1) or 14-3-3 proteins, although the complete interaction network is currently unknown. Therefore, the difference in functionality between PaWRI2 and AtWRI2 may result from distinct post-translational modifications and/or protein interactions.

Deletion of C-terminal Ordered Regions in PaWRI1 and PaWRI2 Improved Lipid Accumulation in N. benthamiana Leaves

Both PaWRI1 and PaWRI2 exhibit the distinct characteristic of having an ordered region at their C-terminus and PaWRI2 had the unique feature of the absence of IDR3. To evaluate the role of the ordered domains and the IDR2 region in PaWRI2, DNA fragments encoding strategic C-terminal deletion versions of PaWRI1 and PaWRI2 were cloned into a binary expression vector under the CaMV35S constitutive promoter and transiently expressed them in N. benthamiana leaves to assess their ability to induce a change in lipid content and fatty acid composition.

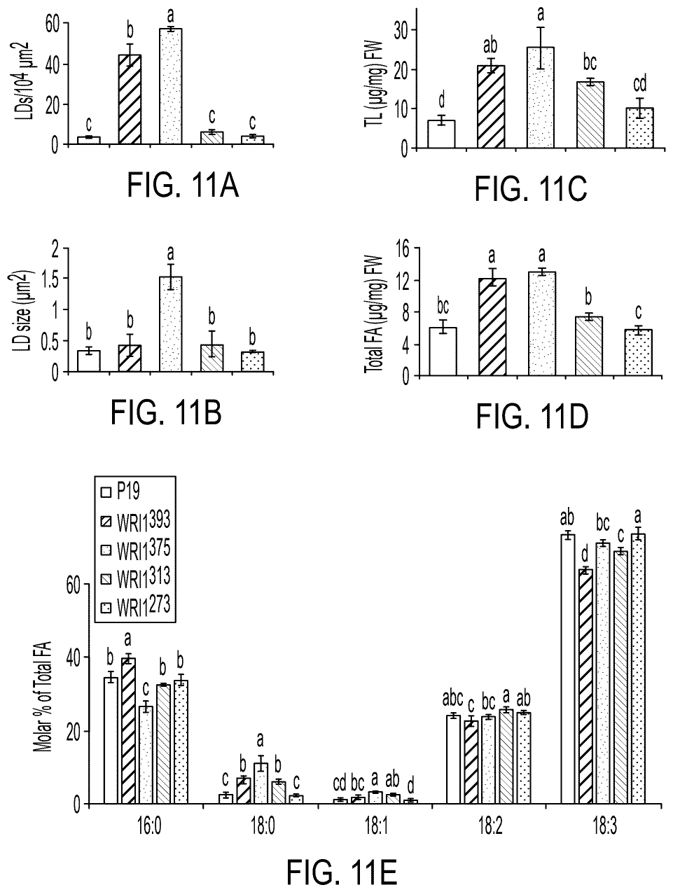

As shown in FIGS. 11A-11E, transient expression of PaWRI1³⁷⁵ improved LD accumulation by 29%. However, deletion of IDR3 alone (PaWRI1³¹³) or combined with the upstream ordered domain (PaWRI1²⁷³) resulted in a sharp decrease in LD accumulation, showing no significant difference from the P19 control. This suggests that deletion of the C-terminus ordered region improved PaWRI1 transactivation activity. Interestingly, an increase in the average size of the LDs by almost 3-fold was observed in leaves expressing PaWRI1³⁷⁵ compared to the control (P19 ) and PaWRI1. This suggests an increase in neutral lipid biosynthesis (mostly TAG) in the leaves. Additionally, there was an increase in total lipid and FA content in leaves, although the differences were not significant. However, deletion of IDR3 further reduced total lipid and FA content, with a significant reduction in FA content observed only in leaves expressing PaWRI1³¹³ . Expression of PaWRI1²⁷³ reduced total lipid and FA content to a level similar to the P19 control. A change in the fatty acyl species composition was observed in leaves expressing PaWRI1³⁷⁵, with a decrease in C16:0 and an increase in C18:3 compared to PaWRI1, while both C18:0 and C18:1 content increased compared to the control (P19 ) and PaWRI1.

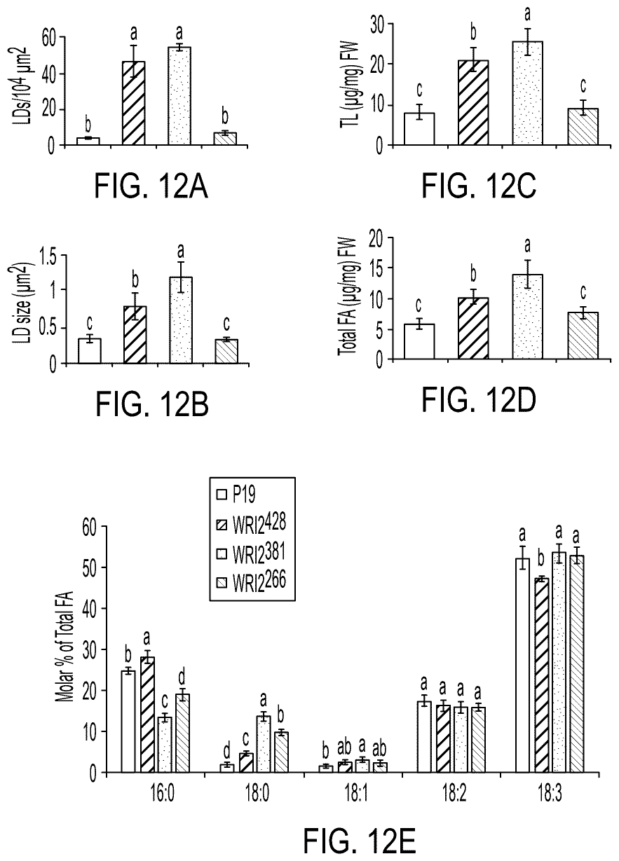

As shown in FIGS. 12A-12E, transient expression of the C-terminal ordered region truncated PaWRI2³⁸¹ increased LD accumulation level compared to the P19 control but showed no significant difference from the wild type PaWRI2. It resulted in an increase in the average size of the LDs compared to P19 and PaWRI2, suggesting that PaWRI2³⁸¹ induced more neutral lipid synthesis in the leaves, organized into larger LDs. Further deletion of IDR2 (PaWRI2²⁶⁶) completely reduced LD number and size, comparable to leaves expressing the P19 control. Moreover, PaWRI2³⁸¹ increased total lipid and fatty acid content in leaves compared to P19 control and PaWRI2, with a ˜20% and ˜30% increase in total lipid and fatty acid contents, respectively, compared to PaWRI2. However, deletion of the C-terminal region including IDR2 (266-428) abolished protein function, reducing total lipid and fatty acid content to a similar level as shown by the P19 control. Fatty acid composition analysis showed a decrease in C16:0 and an increase in C18:0 contents in leaves expressing the truncated PaWRI2³⁸¹ and PaWRI2²⁶⁶ compared to P19 control and wild type PaWRI2, with a more prominent increase observed in the proportion of C18:0. Together, these results suggest that the IDR2 (267-381) in PaWRI2 may be considered the transactivation domain.

### Conclusion

We conducted experiments involving the deletion of the C-terminal domain in both PaWRI1 and PaWRI2. These studies revealed an improvement in transactivation, leading to increased lipid and fatty acid content in N. benthamiana leaves when the ordered fragment at the C-terminus was removed. Specifically, our results suggest that the IDR2 domain in PaWRI2 constitutes the transactivation domain. However, we could not definitively conclude the same for PaWRI1. Based on our observations, it is plausible that the C-terminal region of PaWRI1, spanning from IDR2 to the end of IDR3, may function as a transactivation domain, similar to AtWRI1.

### Example 4: Conclusions

The examples described herein aimed to understand how avocado PaWRI1 and PaWRI2 regulate oil biosynthesis in non-seed mesocarp tissue and apply this knowledge to enhance oleic acid (C18:1) rich TAG content in plants when co-expressed with the acyltransferases PaDGAT1 and PaPDAT1. These findings offer insights into lipid metabolism in avocado mesocarp and the application of these findings to other plants.

The experiments included multigene transient over-expression of all four avocado genes (PaWRI1, PaWRI2, PaDGAT1, and PaPADT1), independently and in combination, in N. benthamiana leaves, identification of affected oil metabolism genes by PaWRI1, PaWRI2, or both, structural analysis of PaWRI1 and PaWRI2, identification of transcriptional target genes by Y1H assay, and elucidation of the functional role of C-terminally located IDRs.

Co-expression of PaWRI1, PaWRI2, PaDGAT1, and PaPDAT1 significantly increased lipid biosynthesis in N. benthamiana leaves, enhancing both lipid droplet number and size, and improving the proportion of C18:1 in the total lipid composition. Although co-expression of PaDGAT1 and PaPDAT1 alone increased TAG accumulation with a preference for C18:1, lipid content did not change significantly due to limitations in fatty acid substrate availability. Further experiments exploring combined strategies showed a substantial increase in lipid accumulation, particularly when all four genes were co-expressed. This combined approach resulted in a significant increase in lipid and TAG content (>260-fold), along with an enhanced proportion of oleic acid (C18:1), demonstrating its potential in enhancing healthy oil production.

Expression analysis of 46 glycolysis, FA biosynthesis, and TAG assembly genes in N. benthamiana leaves affected by PaWRI1 and PaWRI2 revealed selective transactivation of plastidial glycolytic enzymes and suppression of cytosolic enzymes. Although most FA genes were upregulated, low expression of FATA and FATB in leaves posed a bottleneck for further storage oil biosynthesis, suggesting that future strategies should aim at improving their expression. The structural analysis highlighted the presence of one AP2 domain in PaWRI2 and two in PaWRI1, with both showing the likelihood of binding to the target AW-box promoter element. Y1H assays confirmed that both PaWRI1 and PaWRI2 could interact with AW/AWL-box elements in target gene promoters and induce their expression, with PaWRI1 showing stronger binding. Additionally, PaWRI2 was transactivated by itself and by PaWRI1, revealing a complex and interdependent regulatory network of these transcription factors in avocado fruit lipid metabolism.

To further enhance their functions, strategic C-terminal deletion studies were conducted on PaWRI1 and PaWRI2. Removal of the ordered fragments at the C-terminus improved transactivation, leading to increased lipid and fatty acid content in N. benthamiana leaves. The IDR2 domain in PaWRI2 was identified as the transactivation domain, while the function of the corresponding region in PaWRI1 remains to be definitively established. Overall, these findings pave the way for utilizing avocado genes to enhance oil production with desired levels of C18:1 in various plant species.

Aspects of the present disclosure can be described with reference to the following numbered clauses, with preferred features laid out in dependent clauses.
1. A method for producing a lipid or oil in a plant, the method comprising:
   genetically modifying the plant to express a plurality of heterologous proteins selected from PaWRI1, PaWRI2, PaDGAT1, or PaPDAT1, or variants thereof,
   wherein expression of the plurality of heterologous proteins results in a change in the nutrient profile of the plant relative to non-genetically modified plants of the same species.
2. The method of clause 1, wherein the change to the nutrient profile comprises an increase in one or more of quantity of total lipid extracted, total fatty acid content, or amount of TAG and/or a change in the fatty acid profile.
3. The method of clause 1 or clause 2, wherein the change in the nutrient profile comprises an increase in oleic acid incorporation in the TAG.
4. The method of any of the preceding clauses, wherein the plurality of heterologous proteins comprises PaWRI1 and PaWRI2.
5. The method of any of clauses 1-3, wherein the plurality of heterologous proteins comprises PaWRI1 and PaDGAT1.
6. The method of any of clauses 1-3, wherein the plurality of heterologous proteins comprises PaWRI1 and PaPDAT1.
7. The method of any of clauses 1-3 or 5-6, wherein the plurality of heterologous proteins comprises PaWRI1, PaDGAT1, and PaPDAT1.
8. The method of any of clauses 1-3, wherein the plurality of heterologous proteins comprises PaWRI2 and PaDGAT1.
9. The method of any of clauses 1-3, wherein the plurality of heterologous proteins comprises PaWRI2 and PaPDAT1.
10. The method of any of clauses 1-3 or 8-9, wherein the plurality of heterologous proteins comprises PaWRI2, PaDGAT1, and PaPDAT1.
11. The method of any of the preceding clauses, wherein the plurality of heterologous proteins comprises PaWRI1, PaWRI2, PaDGAT1, and PaPDAT1.
12. The method of any of the preceding clauses, wherein the change to the nutrient profile comprises a decrease in palmitic acid.
13. The method of any of the preceding clauses, wherein the change to the nutrient profile comprises a decrease in linoleic acid.
14. The method of any of the preceding clauses, wherein the change to the nutrient profile comprises an increase in the quantity of total lipid extracted.
15. The method of any of the preceding clauses, wherein PaWRI1 comprises a C-terminally modified variant.
16. The method of clause 15, wherein the C-terminally modified variant is selected from PaWRI1³⁷⁵, PaWRI1³¹³ or PaWRI1²⁷³.
17. The method of any of the preceding clauses, wherein PaWRI2 comprises a C-terminally modified variant.
18. The method of clause 17, wherein the C-terminally modified variant is selected from PaWRI2³⁸¹, or PaWRI2²⁶⁶.
19. A method for producing fatty acid and triacylglycerol content in plant non-seed tissue, the method comprising:
   genetically modifying the plant to express a plurality of heterologous proteins in the plant non-seed tissue, the heterologous proteins selected from PaWRI1, PaWRI2, PaDGAT1, or PaPDAT1, or variants thereof,
   wherein expression of the plurality of heterologous proteins results in a change in the nutrient profile of the plant relative to non-genetically modified plants of the same species.
20. A method for producing a genetically modified plant comprising:
   genetically modifying the plant by one or more of Agrobacterium-mediated transformation, biolistic bombardment, protoplast transformation, electroporation, microinjection, PEG-mediated transformation, CRISPR-Cas9, transgrafting, RNA interference, virus-induced gene silencing, and sonication,
   wherein the genetically modified plant expresses a plurality of heterologous proteins selected from PaWRI1, PaWRI2, PaDGAT1, or PaPDAT1, or variants thereof, and expression of the plurality of heterologous proteins results in a change in the nutrient profile of the genetically modified plant relative to non-genetically modified plants of the same species.
21. The method of clause 20, wherein the plurality of heterologous proteins comprises PaWRI1 and PaWRI2.
22. The method of clause 20, wherein the plurality of heterologous proteins comprises PaWRI1 and PaDGAT1.
23. The method of clause 20, wherein the plurality of heterologous proteins comprises PaWRI1 and PaPDAT1.
24. The method of clause 20, wherein the plurality of heterologous proteins comprises PaWRI1, PaDGAT1, and PaPDAT1.
25. The method of clause 20, wherein the plurality of heterologous proteins comprises PaWRI2and PaDGAT1.
26. The method of clause 20, wherein the plurality of heterologous proteins comprises PaWRI2and PaPDAT1.
27. The method of clause 20, wherein the plurality of heterologous proteins comprises PaWRI2, PaDGAT1, and PaPDAT1.
28. The method of clause 20, wherein the plurality of heterologous proteins comprises PaWRI1, PaWRI2, PaDGAT1, and PaPDAT1.
29. The method of any of the preceding clauses 20-24 and 28, wherein PaWRI1 comprises a C-terminally modified variant.
30. The method of clause 29, wherein the C-terminally modified variant is selected from PaWRI1³⁷⁵, PaWRI1³¹³ or PaWRI1²⁷³.
31. The method of any of the preceding clauses 20-21 and 25-27, wherein PaWRI2 comprises a C-terminally modified variant.
32. The method of clause 31, wherein the C-terminally modified variant is selected from PaWRI2³⁸¹ or PaWRI2²⁶⁶.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for producing a lipid or oil in a plant, the method comprising:
genetically modifying the plant to express a plurality of heterologous proteins selected from PaWRI1, PaWRI2, PaDGAT1, or PaPDAT1, or variants thereof,
wherein expression of the plurality of heterologous proteins results in a change in the nutrient profile of the plant relative to non-genetically modified plants of the same species.

2. The method of claim 1, wherein the change to the nutrient profile comprises an increase in one or more of quantity of total lipid extracted, total fatty acid content, or amount of TAG and/or a change in the fatty acid profile.

3. The method of claim 1, wherein the change in the nutrient profile comprises an increase in oleic acid incorporation in the TAG.

4. The method of claim 1, wherein the plurality of heterologous proteins comprises PaWRI1 and PaWRI2.

5. The method of claim 1, wherein the plurality of heterologous proteins comprises PaWRI1 and PaDGAT1.

6. The method of claim 1, wherein the plurality of heterologous proteins comprises PaWRI1 and PaPDAT1.

7. The method of claim 1, wherein the plurality of heterologous proteins comprises PaWRI1, PaDGAT1, and PaPDAT1.

8. The method of claim 1, wherein the plurality of heterologous proteins comprises PaWRI2 and PaDGAT1.

9. The method of claim 1, wherein the plurality of heterologous proteins comprises PaWRI2 and PaPDAT1.

10. The method of claim 1, wherein the plurality of heterologous proteins comprises PaWRI2, PaDGAT1, and PaPDAT1.

11. The method of claim 1, wherein the plurality of heterologous proteins comprises PaWRI1, PaWRI2, PaDGAT1, and PaPDAT1.

12. The method of claim 1, wherein the change to the nutrient profile comprises a decrease in palmitic acid.

13. The method of claim 1, wherein the change to the nutrient profile comprises a decrease in linoleic acid.

14. The method of claim 1, wherein the change to the nutrient profile comprises an increase in the quantity of total lipid extracted.

15. The method of claim 1, wherein PaWRI1 comprises a C-terminally modified variant.

16. The method of claim 15, wherein the C-terminally modified variant is selected from PaWRI1³⁷⁵, PaWRI1³¹³ or PaWRI1²⁷³.

17. The method of claim 1, wherein PaWRI2 comprises a C-terminally modified variant.

18. The method of claim 17, wherein the C-terminally modified variant is selected from PaWRI2³⁸¹ or PaWRI2²⁶⁶.

19. A method for producing fatty acid and triacylglycerol content in plant non-seed tissue, the method comprising:
genetically modifying the plant to express a plurality of heterologous proteins in the plant non-seed tissue, the heterologous proteins selected from PaWRI1, PaWRI2, PaDGAT1, or PaPDAT1, or variants thereof,
wherein expression of the plurality of heterologous proteins results in a change in the nutrient profile of the plant relative to non-genetically modified plants of the same species.

20. A method for producing a genetically modified plant comprising:
genetically modifying the plant by one or more of Agrobacterium-mediated transformation, biolistic bombardment, protoplast transformation, electroporation, microinjection, PEG-mediated transformation, CRISPR-Cas9, transgrafting, RNA interference, virus-induced gene silencing, and sonication,
wherein the genetically modified plant expresses a plurality of heterologous proteins selected from PaWRI1, PaWRI2, PaDGAT1, or PaPDAT1, or variants thereof, and expression of the plurality of heterologous proteins results in a change in the nutrient profile of the genetically modified plant relative to non-genetically modified plants of the same species.


SEQUENCE LISTING (148 sequences)

SEQ ID NO: 1 - DNA, 1182 bp, Persea americana
  source 1..1182: mol_type = other DNA
atggacacatcttctcccctctccaattcaatctcatcttcctcctcctccaccaccaccacctcttcttcttcttcttcttcacctctttctcaacccaccaaaacaaagatcaagcgcactagaaggaatctcaaccctcagaagagccagtttggtgccaaccctggaagaagaagttctgtgtatagaggtgttactaggcatagatggacaggaaggtatgaggcccacctttgggacaagagcagttggaaccctgttcaaaacaagaaaggaagacaagtctatttgggagcctatgatgatgaagaggctgctgctcatacatatgaccttgctgctctcaaatattggggccctgataccatcttgaatttccctctgaatacatatgcaaaagagtttgaggagatgcaaatggtgtctaaggaagagtacttggcttctttaaggagaagaagtagtgggttttccagaggagtttctaaataccgtggggtggcaaggcatcatcacaacggacggtgggaggctaggattggtcgagtctttggaaataaatatctctacttgggaacatacagtactcaagaagaagcagctgcagcatatgacatggctgctatagaatacagagggctcaatgcagtaacaaacttcgatatcagccattacataagatgccaaccacccccacctcaacccccacaaccctccgcccccttccaggaacctcatcatgtggccccacctcagattcaaatccccgaaaactccggtcccatcgatcaaactgcgatcatggaccccctcacagatcagccctggaacctctgcatggaccccaccttcaactccattccggttcccaatatcgccctcgacaaatccattgaattacatgacctgttcaatggcactggctttgaagacaatatagagttcctttttgatggagctgagggtggttttcccactggtcccactactacaggcatccatgaatgtgagggaaatgagatgaaattggatgatggcatttttgagaattcaggcgggtttgagggcgatttcctgaatgatattgaagagggtggtgaatgtgggttgatagttgagaatatgaatgcttctgttgactctatttcctatccgcttcccatcagcatatgttcttag

SEQ ID NO: 2 - protein, 393 aa, Persea americana
MDTSSPLSNSISSSSSSTTTTSSSSSSSPLSQPTKTKIKRTRRNLNPQKSQFGANPGRRSSVYRGVTRHRWTGRYEAHLWDKSSWNPVQNKKGRQVYLGAYDDEEAAAHTYDLAALKYWGPDTILNFPLNTYAKEFEEMQMVSKEEYLASLRRRSSGFSRGVSKYRGVARHHHNGRWEARIGRVFGNKYLYLGTYSTQEEAAAAYDMAAIEYRGLNAVTNFDISHYIRCQPPPPQPPQPSAPFQEPHHVAPPQIQIPENSGPIDQTAIMDPLTDQPWNLCMDPTFNSIPVPNIALDKSIELHDLFNGTGFEDNIEFLFDGAEGGFPTGPTTTGIHECEGNEMKLDDGIFENSGGFEGDFLNDIEEGGECGLIVENMNASVDSISYPLPISICS

SEQ ID NO: 3 - DNA, 1287 bp, Persea americana
  source 1..1287: mol_type = other DNA
atggcttcttctccttcgtcgtcggatcctcctgtgctgaagatcgaagctgctgcagctactggtggtggtggtggaggaagtggaggaggaggaggtggaggaggagggggcggagaagcttcggaagctttaatagcgacggatcgtcttctgttcagaggactgaagaaggcgaggaaggagagggtgtgtacggcaaaggagaggataagcaagatgccgccatgtgctgcggggaaaaggagctcgatttacagaggagttaccaggcatagatggactggacgctatgaggctcacctttgggacaaaagtacatggaaccaaaaccaaaataagaaggggaaacaagtttacttgggtgcatatgatgatgaggaggctgcggctagagcatacgatctggctgctctaaaatattggggtccagggactcttatcaattttcctgttagcgattattcacgagatcttgaagagatgcagatggtttcaagagaggactaccttgcatcactgagaagaaaaagtagtggcttctcaagaggtttctcaaaatatcgtggagtttccaggccacaaagtagtcgatgggagacatccttcgggcgaattcttgggaatgagaatttcaatagcttaaactgcagtactagtgaggatgctgcaactgaaaacgaatatacgggtacctttttccttgagagaaaaattgatctgacaaattacatcaggtggtgggggcctaaaaagatacgtcgatcggatcctatcactaaatcctctgatgaaacacatggtatttcagatgcaggcagtgaactcaaaacatttgaatggccctcgcaacatactgagccatatcagctgccttctctaggcctgtcttgtaaagggaaaccttttgcatcagctatgtctgcctgtcgcatattggcacagtctgctgccttcaagaaaatgcaagagaaagcatcagaagcccaagatggtgagcacacaaacaatataggccatgaaaagacggttctgaaattgtcaagtgttggtgggagtgagagttccggagttggattaagcttgggtgaaatgcctcttcaaaaaacgtcataccctttgggccctttcttatcagctccattgctgacaaattgcagcaacatcgacccgtcgccagactctgcattctggacaaatctaatccagccaacaggactttccctaagcacaacacataggaagaatgagatcagttcaacatatactttcttccgtcaagaataa

SEQ ID NO: 4 - protein, 428 aa, Persea americana
MASSPSSSDPPVLKIEAAAATGGGGGGSGGGGGGGGGGGEASEALIATDRLLFRGLKKARKERVCTAKERISKMPPCAAGKRSSIYRGVTRHRWTGRYEAHLWDKSTWNQNQNKKGKQVYLGAYDDEEAAARAYDLAALKYWGPGTLINFPVSDYSRDLEEMQMVSREDYLASLRRKSSGFSRGFSKYRGVSRPQSSRWETSFGRILGNENFNSLNCSTSEDAATENEYTGTFFLERKIDLTNYIRWWGPKKIRRSDPITKSSDETHGISDAGSELKTFEWPSQHTEPYQLPSLGLSCKGKPFASAMSACRILAQSAAFKKMQEKASEAQDGEHTNNIGHEKTVLKLSSVGGSESSGVGLSLGEMPLQKTSYPLGPFLSAPLLTNCSNIDPSPDSAFWTNLIQPTGLSLSTTHRKNEISSTYTFFRQE

SEQ ID NO: 5 - DNA, 2079 bp, Persea americana
  source 1..2079: mol_type = other DNA
aaagccatcctttttttaactttgtatatatataaacaatactacaaagaaacgaaagtacgaccgctgcatcaaatatcaatatcggtgcgctgattccagattcatatcctcctgaaacgtaaaagatcggattctctccctctctctctctctcgcttctgcagatggcgatctcgactccggaagaaaccctaaccctaacgcaggacagagttccagtctcagatctgcggcgccggccgcctccctccaccgctatctcctccgtgctcgactcctcctccaagacctgcgcgctgccggacggtagctccaaagacgactccagcgacaattccagcgtcggcaacaccttcgacggagaacagaggaatccgtcggtcgacggcggataccggacggataaagtcgacggcgatgatcggacggataaggttaaacgagatgaggcaaggatcgggaacggcgaagatcgtggtaagacggtcgacttcaggttcccatatgggccgtcggttcccgctcatcggagggtggtagagagtcctctgagttcggatttgatcttcaaacagagtcatgcaggccttttcaatctctgtattgttgtgcttattgcggtgaatagcaggcttatcattgagaatttgatgaagtatggcctattgattggatctggattttggtttagctcaaggtcattgaaagattggccattatttatgtgttgcctctctctatcgattttccctctttgcgcttttatggttgagaaattggcacaacagaagttaatctctgaactggttgttgtttctcttcacgtaacaattacaatggctgaaatcatatatccagtttttgtgattcttaggtctgattctgctgtgctatccggcatcacactaatgatcttgacttgcgttgtttggttgaagctggtatcctatgcccacacaaactatgacataagggccttatgtaagtcagctgataagggtgacgcgcctttcaattatgtgagtgcaaatggtgctcacaacatcagcttccagaatttggcatacttcatggcggctcccacattatgttaccagccaagctatcctcgtactacatgcattcggaagaattgggtgcttcgtcaatttattaaattggtgatatttacaggagtcatgggctttatcattgaacaatacattaacccaattgtgacgaactctcaacatcccctgaaaggaaacttcttatatgctgtggagagggtcttgaagctttcagttcctactttgtatgtgtggctttgcatgttctactgccttttccatctatggttgaatattcttgccgagctcctttgttttggtgatcgtgaattctacaaggactggtggaatgccacaacggttgacgagtactggagaaagtggaatatgcctgttcataaatggatgactcgacatgtgtattttccatgtttgcggaatgggataccaaagggagtttcttttgtgattgccttcttcatatctgctgtattccatgagctatgtattggtgttccctgccacattttcacgttttgggcttttatgggaataatgcttcaggttcccttggtcatcattacaaattacctgcaaaataaattcagaaatacaatggtggggaatatgatcttctggttctttttctgcatttatggacaacctatgtgtgtcctcctttattaccatgatgtcatgaaccggagagtgagaacagagtaactcgaacttaaagtccgcgtatgcatctttccattatagcatcaagagagctgagcaattcaagtatgtttcagtcctctacccctgttgttgcacagtagcatttagtgactcactcacatcatgcttaccattgaccataattggttccatagaaatcatggaatgcactgtaaatatttgattgggaggaattttgtaacaagatgcatggaagattgttgttgaatggcttgggtaattttagggtaattttgaatgagcggtactagatcgttcctcaagtatctgatcatcagatcac

SEQ ID NO: 6 - protein, 535 aa, Persea americana
MAISTPEETLTLTQDRVPVSDLRRRPPPSTAISSVLDSSSKTCALPDGSSKDDSSDNSSVGNTFDGEQRNPSVDGGYRTDKVDGDDRTDKVKRDEARIGNGEDRGKTVDFRFPYGPSVPAHRRVVESPLSSDLIFKQSHAGLFNLCIVVLIAVNSRLIIENLMKYGLLIGSGFWFSSRSLKDWPLFMCCLSLSIFPLCAFMVEKLAQQKLISELVVVSLHVTITMAEIIYPVFVILRSDSAVLSGITLMILTCVVWLKLVSYAHTNYDIRALCKSADKGDAPFNYVSANGAHNISFQNLAYFMAAPTLCYQPSYPRTTCIRKNWVLRQFIKLVIFTGVMGFIIEQYINPIVTNSQHPLKGNFLYAVERVLKLSVPTLYVWLCMFYCLFHLWLNILAELLCFGDREFYKDWWNATTVDEYWRKWNMPVHKWMTRHVYFPCLRNGIPKGVSFVIAFFISAVFHELCIGVPCHIFTFWAFMGIMLQVPLVIITNYLQNKFRNTMVGNMIFWFFFCIYGQPMCVLLYYHDVMNRRVRTE

SEQ ID NO: 7 - DNA, 2052 bp, Persea americana
  source 1..2052: mol_type = other DNA
atgttgagacggagaaaagcgactgaatcggagaagaagcccgagtcctcagatgagaataaaacggataagatcgatagcgaaccagacaaggaggaggagaaagagaagaagaagagcagagcagaaacggaggacaagcgtaataagcaccaacatcacagatggtcgtgcgtggacagctgttgctggttcattggctgcatatgttcgatttggtggtttctgctgtttctctataacgccatgccggcgtccttcccgcagtatgtgacggaggcgatcacgggcccgttacctgatccgcctggtgtgaagctgcagaaagaggggctgtcggtaaaacacccggtgctgtttgtaccggggattgtgactgggggactcgagttgtgggaaggccaccattgtgcagatgggttgttccgaaagaggctctggggtgggacctttggagaagtatataagagacctttatgctgggttgaacacatgtcattggacaatgaaactggtctggatccatctggtataagggtcagacctgtcacagggcttgtggcagcagattactttgctcctggctattttgtatgggctgttttgattgctaatttggcacgcattggttatgaagagaaaactatgtatatggccgcatatgattggagaatctcgtttcaaaacactgaggttcgggatcaatctctcagcagaataaaaagtaatatagagcttatggtagctacaaatgatgggaaaaaggtggtggtcattccccactctatgggagttctatattttttgcatttcatgaagtgggttgaagcaccagctccgatgggtggtggtggtggtccagactggtgtgctaaacatattaaggcagtaatgaatattggtggaccatttttaggtgttccaaaagcagctgcagggctgttctctgctgaagccagggatgttgctgtcgctagggcaattgcacctggtgtcttggattctgattatcttggtcttcaaactttacaacatgtgatgcgcatgacccgaacatgggactcaactatgtcaatgattccaaaagggggagacaccatttggggtggcctagactggtctccagaagaaggatatgactgtagtcctaagaagcagaagaacaatgatacacatatttcagctgataatgggacagtgagtgtgagttctcaatctatccatatgaattatggaaggattatatcatttggtaaagatgtggcagaggcacattcatcccagattgagcaaatcgaatttaggggtgctgttaagggcaataagcttgctaaatcaaatgtgtcttgcgatatatggacggagtaccatgagatgggtttgggaggcatcaaagctgttgctgactacaaagtctacaccgccggatccctgttggatctgctcaattttgttgctccaaaaatgatgcagcgtggaagtgctcacttttcatatggtattgcagaggatttggatgacccacagtatgagcactacaaatattggtcaaaccccttagaaacgaaattaccaaatgctcccgatatggagatctattctatgtatggggttggaatacctactgaaagatcatatatttacaaggcatcatccccgactgaatgctacattccctttcagattgatacctcagctgagggcggaaagggtggcagctgtttacaaggtggagtctattcagttgatggggatgagaccgtgcctgttttaagtgcaggctacatgtgtgccaaaggttggcgcgggaaaaccagatttaatccttcagaaattcggactttcatccgggagtataatcatgccccgccatcaactcttttggagggacgaggcacccagagtggcgctcatgttgatataatggggaactttgcattgattgaagatgttataagagtggcagcaggagctacaggtgaagacttgggcggcgatcaagtttattcagatatctttaaatggtccgagaaaatcaaactagaactctag

SEQ ID NO: 8 - protein, 683 aa, Persea americana
MLRRRKATESEKKPESSDENKTDKIDSEPDKEEEKEKKKSRAETEDKRNKHQHHRWSCVDSCCWFIGCICSIWWFLLFLYNAMPASFPQYVTEAITGPLPDPPGVKLQKEGLSVKHPVLFVPGIVTGGLELWEGHHCADGLFRKRLWGGTFGEVYKRPLCWVEHMSLDNETGLDPSGIRVRPVTGLVAADYFAPGYFVWAVLIANLARIGYEEKTMYMAAYDWRISFQNTEVRDQSLSRIKSNIELMVATNDGKKVVVIPHSMGVLYFLHFMKWVEAPAPMGGGGGPDWCAKHIKAVMNIGGPFLGVPKAAAGLFSAEARDVAVARAIAPGVLDSDYLGLQTLQHVMRMTRTWDSTMSMIPKGGDTIWGGLDWSPEEGYDCSPKKQKNNDTHISADNGTVSVSSQSIHMNYGRIISFGKDVAEAHSSQIEQIEFRGAVKGNKLAKSNVSCDIWTEYHEMGLGGIKAVADYKVYTAGSLLDLLNFVAPKMMQRGSAHFSYGIAEDLDDPQYEHYKYWSNPLETKLPNAPDMEIYSMYGVGIPTERSYIYKASSPTECYIPFQIDTSAEGGKGGSCLQGGVYSVDGDETVPVLSAGYMCAKGWRGKTRFNPSEIRTFIREYNHAPPSTLLEGRGTQSGAHVDIMGNFALIEDVIRVAAGATGEDLGGDQVYSDIFKWSEKIKLEL

SEQ ID NO: 9 - DNA, 1128 bp, Persea americana
  source 1..1128: mol_type = other DNA
atggacacatcttctcccctctccaattcaatctcatcttcctcctcctccaccaccaccacctcttcttcttcttcttcttcacctctttctcaacccaccaaaacaaagatcaagcgcactagaaggaatctcaaccctcagaagagccagtttggtgccaaccctggaagaagaagttctgtgtatagaggtgttactaggcatagatggacaggaaggtatgaggcccacctttgggacaagagcagttggaaccctgttcaaaacaagaaaggaagacaagtctatttgggagcctatgatgatgaagaggctgctgctcatacatatgaccttgctgctctcaaatattggggccctgataccatcttgaatttccctctgaatacatatgcaaaagagtttgaggagatgcaaatggtgtctaaggaagagtacttggcttctttaaggagaagaagtagtgggttttccagaggagtttctaaataccgtggggtggcaaggcatcatcacaacggacggtgggaggctaggattggtcgagtctttggaaataaatatctctacttgggaacatacagtactcaagaagaagcagctgcagcatatgacatggctgctatagaatacagagggctcaatgcagtaacaaacttcgatatcagccattacataagatgccaaccacccccacctcaacccccacaaccctccgcccccttccaggaacctcatcatgtggccccacctcagattcaaatccccgaaaactccggtcccatcgatcaaactgcgatcatggaccccctcacagatcagccctggaacctctgcatggaccccaccttcaactccattccggttcccaatatcgccctcgacaaatccattgaattacatgacctgttcaatggcactggctttgaagacaatatagagttcctttttgatggagctgagggtggttttcccactggtcccactactacaggcatccatgaatgtgagggaaatgagatgaaattggatgatggcatttttgagaattcaggcgggtttgagggcgatttcctgaatgatattgaagagggtggtgaatgtgggttgatagttgagaattag

SEQ ID NO: 10 - DNA, 942 bp, Persea americana
  source 1..942: mol_type = other DNA
atggacacatcttctcccctctccaattcaatctcatcttcctcctcctccaccaccaccacctcttcttcttcttcttcttcacctctttctcaacccaccaaaacaaagatcaagcgcactagaaggaatctcaaccctcagaagagccagtttggtgccaaccctggaagaagaagttctgtgtatagaggtgttactaggcatagatggacaggaaggtatgaggcccacctttgggacaagagcagttggaaccctgttcaaaacaagaaaggaagacaagtctatttgggagcctatgatgatgaagaggctgctgctcatacatatgaccttgctgctctcaaatattggggccctgataccatcttgaatttccctctgaatacatatgcaaaagagtttgaggagatgcaaatggtgtctaaggaagagtacttggcttctttaaggagaagaagtagtgggttttccagaggagtttctaaataccgtggggtggcaaggcatcatcacaacggacggtgggaggctaggattggtcgagtctttggaaataaatatctctacttgggaacatacagtactcaagaagaagcagctgcagcatatgacatggctgctatagaatacagagggctcaatgcagtaacaaacttcgatatcagccattacataagatgccaaccacccccacctcaacccccacaaccctccgcccccttccaggaacctcatcatgtggccccacctcagattcaaatccccgaaaactccggtcccatcgatcaaactgcgatcatggaccccctcacagatcagccctggaacctctgcatggaccccaccttcaactccattccggttcccaatatcgccctcgacaaatccattgaattacatgacctgttcaatggcactggctttgaagacaattag

SEQ ID NO: 11 - DNA, 822 bp, Persea americana
  source 1..822: mol_type = other DNA
atggacacatcttctcccctctccaattcaatctcatcttcctcctcctccaccaccaccacctcttcttcttcttcttcttcacctctttctcaacccaccaaaacaaagatcaagcgcactagaaggaatctcaaccctcagaagagccagtttggtgccaaccctggaagaagaagttctgtgtatagaggtgttactaggcatagatggacaggaaggtatgaggcccacctttgggacaagagcagttggaaccctgttcaaaacaagaaaggaagacaagtctatttgggagcctatgatgatgaagaggctgctgctcatacatatgaccttgctgctctcaaatattggggccctgataccatcttgaatttccctctgaatacatatgcaaaagagtttgaggagatgcaaatggtgtctaaggaagagtacttggcttctttaaggagaagaagtagtgggttttccagaggagtttctaaataccgtggggtggcaaggcatcatcacaacggacggtgggaggctaggattggtcgagtctttggaaataaatatctctacttgggaacatacagtactcaagaagaagcagctgcagcatatgacatggctgctatagaatacagagggctcaatgcagtaacaaacttcgatatcagccattacataagatgccaaccacccccacctcaacccccacaaccctccgcccccttccaggaacctcatcatgtggccccacctcagattcaaatccccgaaaactccggtcccatcgatcaaactgcgatcatggaccccctcacatag

SEQ ID NO: 12 - DNA, 1146 bp, Persea americana
  source 1..1146: mol_type = other DNA
atggcttcttctccttcgtcgtcggatcctcctgtgctgaagatcgaagctgctgcagctactggtggtggtggtggaggaagtggaggaggaggaggtggaggaggagggggcggagaagcttcggaagctttaatagcgacggatcgtcttctgttcagaggactgaagaaggcgaggaaggagagggtgtgtacggcaaaggagaggataagcaagatgccgccatgtgctgcggggaaaaggagctcgatttacagaggagttaccaggcatagatggactggacgctatgaggctcacctttgggacaaaagtacatggaaccaaaaccaaaataagaaggggaaacaagtttacttgggtgcatatgatgatgaggaggctgcggctagagcatacgatctggctgctctaaaatattggggtccagggactcttatcaattttcctgttagcgattattcacgagatcttgaagagatgcagatggtttcaagagaggactaccttgcatcactgagaagaaaaagtagtggcttctcaagaggtttctcaaaatatcgtggagtttccaggccacaaagtagtcgatgggagacatccttcgggcgaattcttgggaatgagaatttcaatagcttaaactgcagtactagtgaggatgctgcaactgaaaacgaatatacgggtacctttttccttgagagaaaaattgatctgacaaattacatcaggtggtgggggcctaaaaagatacgtcgatcggatcctatcactaaatcctctgatgaaacacatggtatttcagatgcaggcagtgaactcaaaacatttgaatggccctcgcaacatactgagccatatcagctgccttctctaggcctgtcttgtaaagggaaaccttttgcatcagctatgtctgcctgtcgcatattggcacagtctgctgccttcaagaaaatgcaagagaaagcatcagaagcccaagatggtgagcacacaaacaatataggccatgaaaagacggttctgaaattgtcaagtgttggtgggagtgagagttccggagttggattaagcttgggtgaaatgcctcttcaaaaaacgtcataccctttgggccctttcttatcagctccataa

SEQ ID NO: 13 - DNA, 798 bp, Persea americana
  source 1..798: mol_type = other DNA
atggcttcttctccttcgtcgtcggatcctcctgtgctgaagatcgaagctgctgcagctactggtggtggtggtggaggaagtggaggaggaggaggtggaggaggagggggcggagaagcttcggaagctttaatagcgacggatcgtcttctgttcagaggactgaagaaggcgaggaaggagagggtgtgtacggcaaaggagaggataagcaagatgccgccatgtgctgcggggaaaaggagctcgatttacagaggagttaccaggcatagatggactggacgctatgaggctcacctttgggacaaaagtacatggaaccaaaaccaaaataagaaggggaaacaagtttacttgggtgcatatgatgatgaggaggctgcggctagagcatacgatctggctgctctaaaatattggggtccagggactcttatcaattttcctgttagcgattattcacgagatcttgaagagatgcagatggtttcaagagaggactaccttgcatcactgagaagaaaaagtagtggcttctcaagaggtttctcaaaatatcgtggagtttccaggccacaaagtagtcgatgggagacatccttcgggcgaattcttgggaatgagaatttcaatagcttaaactgcagtactagtgaggatgctgcaactgaaaacgaatatacgggtacctttttccttgagagaaaaattgatctgacaaattacatcaggtggtgggggcctaaaaagatacgtcgatcggatcctatcactaaatcctctgatgaataa

SEQ ID NO: 14 - DNA, 18 bp, synthetic construct
  source 1..18: mol_type = other DNA
gccaagcaagcatttgac

SEQ ID NO: 15 - DNA, 20 bp, synthetic construct
  source 1..20: mol_type = other DNA
gaatgttcacctccctcttc

SEQ ID NO: 16 - DNA, 20 bp, synthetic construct
  source 1..20: mol_type = other DNA
gcccgaggacatgagaataa

SEQ ID NO: 17 - DNA, 18 bp, synthetic construct
  source 1..18: mol_type = other DNA
gtcatctccgccatctcc

SEQ ID NO: 18 - DNA, 22 bp, synthetic construct
  source 1..22: mol_type = other DNA
ggataagcttctcttccatcag

SEQ ID NO: 19 - DNA, 21 bp, synthetic construct
  source 1..21: mol_type = other DNA
ctttgactctccactgacttc

SEQ ID NO: 20 - DNA, 22 bp, synthetic construct
  source 1..22: mol_type = other DNA
ctactcgtaggttcagcattac

SEQ ID NO: 21 - DNA, 20 bp, synthetic construct
  source 1..20: mol_type = other DNA
caaccgtgtcaagagaatca

SEQ ID NO: 22 - DNA, 18 bp, synthetic construct
  source 1..18: mol_type = other DNA
gaagactgcatctggcaa

SEQ ID NO: 23 - DNA, 19 bp, synthetic construct
  source 1..19: mol_type = other DNA
tagcaccagcagcatagta

SEQ ID NO: 24 - DNA, 22 bp, synthetic construct
  source 1..22: mol_type = other DNA
caagagagttggaagagatgag

SEQ ID NO: 25 - DNA, 22 bp, synthetic construct
  source 1..22: mol_type = other DNA
gtagtctgtctccaaagcatag

SEQ ID NO: 26 - DNA, 20 bp, synthetic construct
  source 1..20: mol_type = other DNA
attccactgccaacagatac

SEQ ID NO: 27 - DNA, 22 bp, synthetic construct
  source 1..22: mol_type = other DNA
ctagtgaagctaagcccataag

SEQ ID NO: 28 - DNA, 20 bp, synthetic construct
  source 1..20: mol_type = other DNA
ctgctcctccagttcaaatc

SEQ ID NO: 29 - DNA, 20 bp, synthetic construct
  source 1..20: mol_type = other DNA
tgtcaccaaccttcacaaat

SEQ ID NO: 30 - DNA, 18 bp, synthetic construct
  source 1..18: mol_type = other DNA
aaccacagttgcctcctg

SEQ ID NO: 31 - DNA, 20 bp, synthetic construct
  source 1..20: mol_type = other DNA
ccctttctggaccttgtctc

SEQ ID NO: 32 - DNA, 20 bp, synthetic construct
  source 1..20: mol_type = other DNA
gctgaggaaaggaactgaaa

SEQ ID NO: 33 - DNA, 20 bp, synthetic construct
  source 1..20: mol_type = other DNA
gcaccactacacagagattg

SEQ ID NO: 34 - DNA, 20 bp, synthetic construct
  source 1..20: mol_type = other DNA
ctagagctcagagatggtga

SEQ ID NO: 35 - DNA, 21 bp, synthetic construct
  source 1..21: mol_type = other DNA
tccatcaatctccagcataac

SEQ ID NO: 36 - DNA, 23 bp, synthetic construct
  source 1..23: mol_type = other DNA
cctatcgatttgagaggtaagag

SEQ ID NO: 37 - DNA, 20 bp, synthetic construct
  source 1..20: mol_type = other DNA
gacccatgtaccaacaagaa

SEQ ID NO: 38 - DNA, 21 bp, synthetic construct
  source 1..21: mol_type = other DNA
catctcctgccttacaatctc

SEQ ID NO: 39 - DNA, 19 bp, synthetic construct
  source 1..19: mol_type = other DNA
ggacaaggccaacagtatc

SEQ ID NO: 40 - DNA, 20 bp, synthetic construct
  source 1..20: mol_type = other DNA
catgactgtggacatggaag

SEQ ID NO: 41 - DNA, 22 bp, synthetic construct
  source 1..22: mol_type = other DNA
gattcgtcagcttcaacatttc

SEQ ID NO: 42 - DNA, 22 bp, synthetic construct
  source 1..22: mol_type = other DNA
cagaggataggaaacggttaag

SEQ ID NO: 43 - DNA, 22 bp, synthetic construct
  source 1..22: mol_type = other DNA
gcccgagagtatatgaagttac

SEQ ID NO: 44 - DNA, 19 bp, synthetic construct
  source 1..19: mol_type = other DNA
caaggcgtcttcagaaagt

SEQ ID NO: 45 - DNA, 21 bp, synthetic construct
  source 1..21: mol_type = other DNA
cagtcccagcctagaataatg

SEQ ID NO: 46 - DNA, 20 bp, synthetic construct
  source 1..20: mol_type = other DNA
ccttcagggagacagcttta

SEQ ID NO: 47 - DNA, 20 bp, synthetic construct
  source 1..20: mol_type = other DNA
cacccatgtacaggagctat

SEQ ID NO: 48 - DNA, 20 bp, synthetic construct
  source 1..20: mol_type = other DNA
gcccgtgatgaatctgtatg

SEQ ID NO: 49 - DNA, 20 bp, synthetic construct
  source 1..20: mol_type = other DNA
gaaggatcgtatggcctagt

SEQ ID NO: 50 - DNA, 21 bp, synthetic construct
  source 1..21: mol_type = other DNA
cattgttgacgattccttcac

SEQ ID NO: 51 - DNA, 20 bp, synthetic construct
  source 1..20: mol_type = other DNA
gcacagggatatagcaagag

SEQ ID NO: 52 - DNA, 20 bp, synthetic construct
  source 1..20: mol_type = other DNA
gatcagcaggtattggaagg

SEQ ID NO: 53 - DNA, 20 bp, synthetic construct
  source 1..20: mol_type = other DNA
caatggtactccagttgctc

SEQ ID NO: 54 - DNA, 20 bp, synthetic construct
  source 1..20: mol_type = other DNA
tctttacctttgcccagttc

SEQ ID NO: 55 - DNA, 20 bp, synthetic construct
  source 1..20: mol_type = other DNA
aatgctgggtacctcaattc

SEQ ID NO: 56 - DNA, 20 bp, synthetic construct
  source 1..20: mol_type = other DNA
cctcttcctcttcctcttct

SEQ ID NO: 57 - DNA, 18 bp, synthetic construct
  source 1..18: mol_type = other DNA
tcactaggaccagccttt

SEQ ID NO: 58 - DNA, 21 bp, synthetic construct
  source 1..21: mol_type = other DNA
gtgtcaccagaagaggttaat

SEQ ID NO: 59 - DNA, 18 bp, synthetic construct
  source 1..18: mol_type = other DNA
ccaagcaatgccctatca

SEQ ID NO: 60 - DNA, 18 bp, synthetic construct
  source 1..18: mol_type = other DNA
ccaagcaatgccctatca

SEQ ID NO: 61 - DNA, 21 bp, synthetic construct
  source 1..21: mol_type = other DNA
gacgtactctgttctttccac

SEQ ID NO: 62 - DNA, 19 bp, synthetic construct
  source 1..19: mol_type = other DNA
ttatctgcgccttcaacag

SEQ ID NO: 63 - DNA, 21 bp, synthetic construct
  source 1..21: mol_type = other DNA
gggtagaaaggacgataatgc

SEQ ID NO: 64 - DNA, 20 bp, synthetic construct
  source 1..20: mol_type = other DNA
gatccaagggcaaagatcag

SEQ ID NO: 65 - DNA, 21 bp, synthetic construct
  source 1..21: mol_type = other DNA
gaccggtttatcggagtttat

SEQ ID NO: 66 - DNA, 20 bp, synthetic construct
  source 1..20: mol_type = other DNA
acatactttccaggctttcc

SEQ ID NO: 67 - DNA, 22 bp, synthetic construct
  source 1..22: mol_type = other DNA
gagagtatcagtgggagagtta

SEQ ID NO: 68 - DNA, 18 bp, synthetic construct
  source 1..18: mol_type = other DNA
gcggaagcaatcctgaaa

SEQ ID NO: 69 - DNA, 19 bp, synthetic construct
  source 1..19: mol_type = other DNA
cgttgagaagggcttgaaa

SEQ ID NO: 70 - DNA, 21 bp, synthetic construct
  source 1..21: mol_type = other DNA
caacaccctgtctgtcatatc

SEQ ID NO: 71 - DNA, 19 bp, synthetic construct
  source 1..19: mol_type = other DNA
catcagatgctacgccttc

SEQ ID NO: 72 - DNA, 20 bp, synthetic construct
  source 1..20: mol_type = other DNA
gtgatcttccgctagtttcc

SEQ ID NO: 73 - DNA, 23 bp, synthetic construct
  source 1..23: mol_type = other DNA
cctatcgatttgagaggtaagag

SEQ ID NO: 74 - DNA, 20 bp, synthetic construct
  source 1..20: mol_type = other DNA
gacccatgtaccaacaagaa

SEQ ID NO: 75 - DNA, 19 bp, synthetic construct
  source 1..19: mol_type = other DNA
cggctcacttcttgttctt

SEQ ID NO: 76 - DNA, 18 bp, synthetic construct
  source 1..18: mol_type = other DNA
ccacgacaacacgcttat

SEQ ID NO: 77 - DNA, 19 bp, synthetic construct
  source 1..19: mol_type = other DNA
cttgacttcaggtgccttt

SEQ ID NO: 78 - DNA, 20 bp, synthetic construct
  source 1..20: mol_type = other DNA
gcttgtccaacatccttagt

SEQ ID NO: 79 - DNA, 20 bp, synthetic construct
  source 1..20: mol_type = other DNA
ggtgcgccaagcatattaaa

SEQ ID NO: 80 - DNA, 18 bp, synthetic construct
  source 1..18: mol_type = other DNA
tatagccctggcaacagc

SEQ ID NO: 81 - DNA, 18 bp, synthetic construct
  source 1..18: mol_type = other DNA
gctgctttgtgggatctt

SEQ ID NO: 82 - DNA, 20 bp, synthetic construct
  source 1..20: mol_type = other DNA
catctaccttcagaccagga

SEQ ID NO: 83 - DNA, 19 bp, synthetic construct
  source 1..19: mol_type = other DNA
cgtgctgttccgagtaatg

SEQ ID NO: 84 - DNA, 21 bp, synthetic construct
  source 1..21: mol_type = other DNA
gcttctcagtaatgcgattga

SEQ ID NO: 85 - DNA, 19 bp, synthetic construct
  source 1..19: mol_type = other DNA
gcctctacagagggagaat

SEQ ID NO: 86 - DNA, 18 bp, synthetic construct
  source 1..18: mol_type = other DNA
aagaccctgagccttgta

SEQ ID NO: 87 - DNA, 24 bp, synthetic construct
  source 1..24: mol_type = other DNA
ggtcattaatttcattcaggtgga

SEQ ID NO: 88 - DNA, 20 bp, synthetic construct
  source 1..20: mol_type = other DNA
ccttggttgcaaagccataa

SEQ ID NO: 89 - DNA, 21 bp, synthetic construct
  source 1..21: mol_type = other DNA
gcccactctcctaattctgat

SEQ ID NO: 90 - DNA, 19 bp, synthetic construct
  source 1..19: mol_type = other DNA
tgctgttctgatggaacct

SEQ ID NO: 91 - DNA, 19 bp, synthetic construct
  source 1..19: mol_type = other DNA
cttgacacaaagggtcctg

SEQ ID NO: 92 - DNA, 21 bp, synthetic construct
  source 1..21: mol_type = other DNA
gcatgtctcctacttcaacat

SEQ ID NO: 93 - DNA, 20 bp, synthetic construct
  source 1..20: mol_type = other DNA
gtggtggttcgactatgttt

SEQ ID NO: 94 - DNA, 20 bp, synthetic construct
  source 1..20: mol_type = other DNA
aatccacatctgttggaagg

SEQ ID NO: 95 - DNA, 20 bp, synthetic construct
  source 1..20: mol_type = other DNA
ctgccattaccagaggattt

SEQ ID NO: 96 - DNA, 20 bp, synthetic construct
  source 1..20: mol_type = other DNA
acgttcaaggtgtcgaataa

SEQ ID NO: 97 - DNA, 19 bp, synthetic construct
  source 1..19: mol_type = other DNA
agatagcaggaagcaagga

SEQ ID NO: 98 - DNA, 23 bp, synthetic construct
  source 1..23: mol_type = other DNA
ccttctcggaatatgggaaatag

SEQ ID NO: 99 - DNA, 19 bp, synthetic construct
  source 1..19: mol_type = other DNA
tcaagtctttctcgactgc

SEQ ID NO: 100 - DNA, 20 bp, synthetic construct
  source 1..20: mol_type = other DNA
tcattgaagaccttcatccc

SEQ ID NO: 101 - DNA, 19 bp, synthetic construct
  source 1..19: mol_type = other DNA
gagaagctatgcaggatgc

SEQ ID NO: 102 - DNA, 21 bp, synthetic construct
  source 1..21: mol_type = other DNA
cacttccactcctttaagacc

SEQ ID NO: 103 - DNA, 22 bp, synthetic construct
  source 1..22: mol_type = other DNA
gatctctttcagagaggaaagg

SEQ ID NO: 104 - DNA, 20 bp, synthetic construct
  source 1..20: mol_type = other DNA
ggcttggatgaagtagaagc

SEQ ID NO: 105 - DNA, 21 bp, synthetic construct
  source 1..21: mol_type = other DNA
tttacagagggagattcaagc

SEQ ID NO: 106 - DNA, 21 bp, synthetic construct
  source 1..21: mol_type = other DNA
aggaactcgttcattaatccc

SEQ ID NO: 107 - DNA, 23 bp, synthetic construct
  source 1..23: mol_type = other DNA
gtcaaaggttgagaatcaagagg

SEQ ID NO: 108 - DNA, 20 bp, synthetic construct
  source 1..20: mol_type = other DNA
gcatctgagttgcagttacc

SEQ ID NO: 109 - DNA, 20 bp, synthetic construct
  source 1..20: mol_type = other DNA
atggctctgaaactcaatcc

SEQ ID NO: 110 - DNA, 20 bp, synthetic construct
  source 1..20: mol_type = other DNA
catttccaccttccacagaa

SEQ ID NO: 111 - DNA, 19 bp, synthetic construct
  source 1..19: mol_type = other DNA
gcaactggaaatgcaatgg

SEQ ID NO: 112 - DNA, 19 bp, synthetic construct
  source 1..19: mol_type = other DNA
tcgcagttcattcttcacc

SEQ ID NO: 113 - DNA, 20 bp, synthetic construct
  source 1..20: mol_type = other DNA
cgagtctgatgttgatgtgg

SEQ ID NO: 114 - DNA, 20 bp, synthetic construct
  source 1..20: mol_type = other DNA
tgttccacactgatttcacc

SEQ ID NO: 115 - protein, 500 aa, unidentified
TTCCATCGCCCTCGCCACCTACAAAGACTTCTCACTTCTCTCCTTCAAATCACACACACACACACACCCCTCTCTCTCACTTCCCTCCAGTTTCTACTATCGATCTCAATCTTTCAGATCGAACTTCTTACTTCATTTCCCCACCTTTTTGATTCGTGCGTCGTCAATCGTCTTCTAGCGTTCTTGTTTGCTCAAGTGTCGTTTTTTGCAATTAATTTGCTTCATTTGCATCGTCTGATCTGATTCTCGTGGAAAAGTGGGATTGTTTTTATTTTATTTTATAGAATGTGCTTTGTTCTTCCGATTTTCATCAGAATCGGAGTTTTTAGGTTGATTTCTGAGTAGATCCTGTGATTTTGCTTCTTTTGATCTGATTCTAGGCAAGAAGAAACGTTTTAGGGCTTCTTCTTCTTCTTCTTCTCATTTGTTGCCTTCTTGTATTGATTTTTTCTACTAAATCCGAGCTTTTCTGTTGGTTTCTCTTTCTTTTGATCGAGAAT

SEQ ID NO: 116 - protein, 756 aa, unidentified
GGGTCATTACTTCCCTCAAAACAAGAGCTGCATCGTCATATTGATCATGTGTGAGAGAAATGCTACAAATCTGCACTGCATAATCCAGCTGCAAAACATAGTAAAAAAAGAATTAGATAACTAAGTACATCTTTAAAACTAGACTCCCCAAGTAACTCACAACTCAGGCAAGTATGGCTGAGTTCTTGACCGAGTCGCTTGACTCTGCCTTATGAGCCGATTGAACTAGGTTTTGAAGCCTCACAAATCACAATGCATACCTCTTCAATCGACTGAAACAATTTGTTGGGATCAGTCATTTTCACCAAGAAGAGGGAATGAGGATGAGTTCCATTTATTTATAAGTGTACACCGCAGGCCCTTAACATTATAAAACAAAGGTTGATAGAGACGCTATTAGAAACTGACTAAAAAAAACACGTTTTCGGGGTGCAACGTCATTTAAAATCTATTTTTTCTATCCACAGGTGACCTAATTCTTTTTTCTTTTTTTTTTTTTTTTTTTTTTCTAATCCAAGGAAGATTAACTAAAACTTTCTTAGATGACGACGAAGAAGAAGAAGAAGAAGATTAAGAACGCCTTGTAGTTTAATCTCTCACAGCAATAATCCTGTTTCTCTCTTCGAAATGCTGCCACTCGTGCTTTTTCCTTGGAATCTACAGGTCCTTTACCTCTCAAATCCATCACTATAAAGAAGAACGTGATCTCTGCATTCGAATATCAATTCATCTCTCTCGCTCTCTCTCATCACCATC

SEQ ID NO: 117 - protein, 500 aa, unidentified
CCTTGCACAGCCAGAGCATTTTTTTGAGACGTCTAAAGATGTATATTCAGGCATGAAGTGTGGTAGAGATCGATCCTTGCACAGCCAGAGCACTCTTTGGAGACAACCAAAGATGCATGATCAGGCATGGAGCGTATAGACAGAAGGGCAGGCAAAACATGGAGGACCCTGGGATGGTAGATTAGGCTAGGTGGGGAGTAAAATAGTATTCCACTCTCCCATCCGAAGAGGAAAAATACGTGTTCTTCATGTGGGACCCACTTTAGGAGTATGAGTTCAGATTCTAGTCTGGTTTGGGATACGTAAGCCAGCATGTGACTAACAGTAACATATCGTAGTTTTTCTCCTCTCTGTCCACCTACGATTCCAACCTACACAAGTACCAAGACACCCATAAGCCTCCAATCCATCTGAACCAACCTCCCTCTTCTTCCTGGTATTTGATCCTTTGGGAAATAACCCAACAATACCAGAGAAGAAGAATCATCTCCTCTGCTTCA

SEQ ID NO: 118 - protein, 500 aa, unidentified; identical to SEQ ID NO: 117

SEQ ID NO: 119 - protein, 768 aa, unidentified
CCACAAATATCTCCAATATTTTTAAAACACAACAATATTACAATATTCATTACAAACAAAGATTTTTCTTAAAACCACTTTCAATGTAACATCAAAACTTCAAAAATACCCTAAAAAGATGCAACCACCAGAATAACACGAAAATATACAGAAAATCTTAAAATATTGTAACATTATTGATTTTGTCCTTATTTAATATTTTTCACTATTTTGAGTATGGAAACTCAAAAATTCCAAAAAAAATAAAATTCAATTAGCCTTTTTTCCTGTGTGCTTAAAAAAAAAAAGGCCAAAAGGGTAGGGTAGTTGGCCAGAACCACAATTCAAAAACCTATTCAGGCAAAAGTCTGAATTTCACATGCTCGGAGGAATAGTCGATTCTCCACGAGGCCGGATACCTAAAATAAATAAAAAAAACAGCAAAAGGAAAAAGAAAGAAAGAAAGAAAGAAAAGAAAAAAAAAAAAAAAAAAAAAAGTGTGCGCTGAAACCCGGAGCAATGGGTCCTGGTATTCAGCCCACCGAATCGCCAAAACACTCGAATATGCGGTCACAGATGCCACTTGTGCGACGGCAAAAAAGCATGCAATCACGAGGATGAAAAAAAATCCGCTCATCTGTGCCGTCTAAATATCACACCCTAAACCAAATCCAACGGCCACACACAAAGAAACCTCTGGCTCTCTCCTGTATAAACCCTCATCTCAGTCCCGTCCAAGTTCGTAGAAACCACACTCGAATTCAAAGAAAGCAAGCTTGATTGAGAGAA

SEQ ID NO: 120 - protein, 779 aa, unidentified
TATAGAGCACTATACAGCAAAAGCAACAAAGGACGCACATCCATAAAAAAAAATGGCATTACCTTTAAGCAAATCATTAAGAATGATCATAGAAATGATAACAAATTGTAGTTAGTCTAATGATGGTTTAGCAGCAAGGCATCTTCATTTTTCTTATGGACAAAATATCCTATAATAAAATCTCATATAAATGAGATTATTAAAAATCTATTAATAAATCATAAATTAACTTCTAAATAAACACAAAATAACACTAAAATTAGGTTTCTACATAAATTCCTATTCCTCATTTTTTTTTCAATCCATACTATCACAATCCCCTAGCAAAATAAATAAATAAATAAAAATCACCCACCACGATATTTTCAAAATATCGCAATCCCACAGACCAAACAAACCCCCCACATTTGTCGCCGGTGCCCAGTGGGCCTGATCATGTCAGAACCCCAAGTACCTGACCACCGCCACTGCATTTCTACTTCTATACTCATTGAGGAAGTGTTCAGACGACATACGTCGGTCCAAACCTCTCACGATTCACAACCCTAACTCCACGCCTTCTAGAAGCTCTGATGAAAACTCCAGCCGCTTCGTCGACGGTTCTGATTAGATGGACCCCACTTCACCCAATCAAATCCCAATCGCGCACGTGAGACAGCACCGTGGGCGGAGAAAACGAACGCTACTTTGTTTACGTTCTCCGCCCTCCCCCGCACAATAAAAATCCAATCCTTCCCTCTTCCTGCCCTGCCTGCTCTCTCCTCTCTCGTTCTGTTTCA

SEQ ID NO: 121 - protein, 739 aa, unidentified
AAATCACATTGAATTTCTTAATGGCAGAAGTTGTTTGAAAGAGATCACATGTGCGTGGAGTCGAGTTGGGATCATCAGTCTCTAAGTGGGAGATTGTTATGTGTGGAGCTTGATTACACCCGGGTCAAATTGTTAACTTAGGTCTACTGTCATGGATCCAACTATTACCCTGCACTATTTTGTAACCCTAGCATAGCCACTAAAGATCAATGTATATTGTATTCACATCTTCTGCTTTTGAGAATAGTGGAATTGGTATGATCAGTGGACGTGCCTTATGATCAAGGGAGTCATGTAAATCTGTGTATCTTCTTTGTGTGATTTGATTGCATCTTTTGATCTTTATTTTCGAACACAGTGATCCTGACCATCCAACATCACCTAATCCATCCAGAGCAGAGTGGGGGCAAACCACCTGAAAGAATGAGATCGAGGAGCACCAAATAGGTATTTTTTCCCTCTCGTGCTTCACTATTTGCTGATTCACGTCCATTTTACTGCAGGATTTAGCCAACAAACATACATAAGAAGAAATGGAAAGAAAAAAAAGACTAGACGAGCAAAGATCAAGGAGAGAAATTGTTGGGTGACTAACTTCGGTTTCTACGCCTATATCCTTTGTTTTCTCCGCCCTCTCCCTTCTTCCCGATTCTATATATCATCTTCTCCTCTTCTCTCGCTCGAAGAAGAACAAGAAAGAAAGAAAGCAAGCGTCTTTCTTGCTGTGTAGGAAATTAAA

SEQ ID NO: 122 - protein, 774 aa, unidentified
CACACGCATCATCATCATCATCAACGCCTTGTCCCAATTACACAGAAAAAACATTAAAACACACATACACAGGGAAAAAAAACAAAAATTCTATGCCATCCCTTTTTTTTTTTCACTTTAAAAAAGTCACAGATGTTAAAGAAACCCTAAATTATGAGTTGAGATTTGACTGTTTGAGTAGGATTTTATATGAATTATATTTTATTCTAGGAGGTTGGCATTAGGTCACCAACCTTGAATAAGTAATGCAAGTATAGAAAAATAACTTCAATGACAGTCCAATCACTGTTACTAAAAGCTTGACAAATTGATCATAGAGCGGTTCCACATTCACAGCCTATAAATCAGCACTCTTGATTTGATGGGCCCACACCAAATCAAACTGAAAATTGTATCCTATCACTATTGTAAATTTGGTAGTTTTCTTCCTCAACCGTCCATCCCTTGATCTTGATCTTGATCTTCGCCATTGATTGATGGGCGCCATCACAGATGACTGTATCGAACTGTATAGATAGACACCCCTGTCTGAAAAACCGAAGAAAGCTGGCGATGGAAACAATGCTGTGAGTGAGCCAAGAGAGCGTTTTGAGAAAAGAGAGCGCATTAAAATCCCCGAACAACAGCAAATAGGGCCGAGAGAAGAACACAAGTCTCATCTCTCTCTATTTCTCCGCCCTAACTACATATAACCAAACGCTCCCCAAACCCTCTCGATTCCATTCCAACTCTCGATCTCGATCTCGATCTCGATCGATCCATTTGGACTCGGAA

SEQ ID NO: 123 - protein, 780 aa, unidentified
ACGTACCCTAAATATGTATGTAAAAATATAAGGGCTAAGCTTATAAATTTATTAATTCACTAGTTTCTATATTGAATGCTCAACTATTTCAATTTGATCAACTTTTTTCAAATTCATATTCAATTGTTAAGTAAATCACTTGTAAATATAATTAAAAGTATTAAGATATATATCGTTTTCAAAATCTTATAATCATCAATGGCCAAAATCCGATTTTTGAATTTTTTTTCCTTCTCTGTGAGTATATCGGGTTAAAATTCATTCACCCACACTAACACATATACTCTAATCCCTAATAAATTAATTAACTTTTCATGTTTTCAATATATAATAGCTCAGCGGTTAAGCATTTCTTTCGGTAAAGTCACTAAAAATTGAATTCCTATTTGAGTTTTCAACTTTTCATCCATCCAGGATGGGGAACTTTTTATGTATTCTTGTGCATTTGCATGAGATTTACCAAGAAAAAAACCGGGTTAAAATTTAGAGTTGGGCCCACCCCTATAGATCCAGAATATCCTCCGAACAGGGCTCAGAGAGTCCCTTCTTCAATCTCTATAAATATACACAGAAATGCTCAGAGCCAAGCACAAACCGCCAGTATCTTCCTCCTCCTTCTTCTTCTTGAGATTCCTCTTTGAATCTCATCTCAGCTGACAAAACACAGTGTTATTTGTGGGTGAATGTGATTGAGTTCCTTCTCTTGATTTCTTTTGAACTGCAAACATTTGGGTTGTAATCTAGGGTAGATTTGCTTTCATCTTGTCTTTTGATTTGGAG

SEQ ID NO: 124 - protein, 500 aa, unidentified
CTCTTGGCTATTCATGCTGCTTTTCTGAATTAGGCTTATCTTAAAGGACCTTACTCCATTTCTGGTCAAAAGAAATCACGAAAGGTCTCAGCATTCTAGTTGTGTGATCTTGGGTTTGAGCCTGCTCGGTTCACTGGCATTTTTCCTGCTTGACCATAGTGTAAAAATATTAACAGATAGAAACAGGCTGTGTGAGCTGTTGAAAGCTTATTATTATTATTATTAGCACTAAATGTTTCATACTTTTTCCTACTTTTGATTGTTCAGTATTGCTTGAGGTTGCAATAGTGAGCACCTTCTTCCACTAAGCCACAGGCTTGTCTGTTGAAGGATTTTCTTCTGTTTTTAACTTTTAAGTTATATCTTATTTATTCAGCAAAGATGCTTGTGCGTTTGTTCAAGACAGTGGATACTTAAATGCTTCATACTAATTACAAAAAAAAAGGATTCTTAAGCATTGTTTGTTCTCCCATTTTCAGGCTCTTCTTCACTCTTCTCAC

SEQ ID NO: 125 - protein, 1733 aa, unidentified
ACAATTTTACAAATTGAAACCAATTTTTCTTAGCTATATGCAACAAAACGAACCAATAATGATGTGAAGTCCTCTTTGAGAAATCCTTAGGGGCAAGAAAGAATCCTTAAATATGTAGATTCCTCACAGGTTACCAAACGTTCTTTGATACTTGTTTGTTTGAAATTTGAGACACAAAGAATTATAAATTATCCAAATATAATTTCTCACTATAGTTTTTCTAATTCTTGGCAAAACTCAGAAAACTAGGAAAACCTAAAGAGATAAAAGATAACATTTGATATTTGATGTTAGGAGAACAACTCTCTCCCACAGTTTTCACCTAGCTACTTGGGGATCAATTGTAGGCGGACTATGTTCTCTCACCCAAGGGCTCTTCACTTCAGCCCAGGTCTTCTTCGCTCACCCAGGGGGTCAAAGGGGTCTAGCCCCTTTCAGGGTGGGGTAAATAAAAAGGGAGATTTGAAGTGAGAAATTCCCCCAAGTATACCCTTGAATTTTTCTTGCAAACAAAGGCCCAAAGGCAAAACCACAAGCAATGAATAAGCTCTAGCTAGTAGCTACCCGTGACTGGCACGGCAGGGGTTGTTTTGCTCATTCACAAGGTCTGTTTCACATCCCACCCTTCCTTCTCTCTGCATTTGAATGGCCCCCTTTAGATCTCTATACGGCCATGATCTAGCGGACCCTGAGAACCATTTGCATGTTAGTCTCGTGTCGTGTGCCTCTCTCTCTCTCTCTCTCTCTCTCCATCAAAGACACCGACAAAGGGAAGTTTTTACTCCAACCCAGATCAAAATCCGTTTCCTTCCTGGCCTTTCTGTTGTCTTTTTCATTGATTTTCAAATATCTTTGAAAAACCCACCGAAACCATATTCAATTGCAGCTGTAATGCCGGTTCTCTCCTTTAAAAGGCCTGCTTTTTTTGGTGGGTTTTGGGTGAGAAAAGCCCAGGAAAACAAGACGCCCTTATTGACATTACAAGCTCTTTGGTGTATCCGGAAAGAAAGTCTTTCCCCTTTCTGATTTGAATTTTAGTTGGGTAGATGACCCAACTCCCAACTGTTTTTTTTTTTTCACAGGTACGTTGCGATATATTTGATGTTTTGAGAATGTGTTTTTATAGTATTTGAAGGTGATTTGAAATCGACCCAGGGAGAAATTTTGTTGCTTTTTGTTTGTTTGTGCAGAGTTTGTTTGCTGTAGAGAGGAGGACGAGAAGATGTGTATGAGGGTCTGATTGGAGAGATTGGTTTTTGTTTTGATGAAAAACCCACATAAAGGAATGAATTCTTTTCATTTTTTGTGAGGATTTGGAATTGGTTTTGTTAGCAAAAACGATAGAATCTCTGTAGTGATTGTGGGATACAAATCTCTAGAGAAAACCCAGTTGAAAAAAGAAAGTATTTTGAGCTTCTGAATTTGAATTTTTGATTGTCATGTTGTGAATTGTTCCAAAAACCCACAAAATAAACTGCAATTTGTTTTTTTGTTCGTGAAGATTTGGTTTTTCTAGCAGAAAGGATTGTTTCTATCTTTGAACCAACCGAGATTTCAATCTTTATATTTGTGTATCATTTTGTTCAGAAAACCCTCAAACAAATATAAATCGGCTTTGATAGTCCAGGATTTCTGAGTTGGAATTTTAGAGGTATTTGAATACTGGTTAAAAAAGCAAACTTTCCAGGCCAATCAAAATCTCTGAGTACTGGGGGGTGATTTCAATTGGAAAG

SEQ ID NO: 126 - protein, 526 aa, unidentified
AGGTCTTCTGCCCATATCTGGCCCATTGACAACTCTAGTTTGGGTAATCTGAATTTCAAGTTGGGCTCAGCATCCCCGACCTATTGCATTCGACAGGGAAAAGAAAATTTTCCTCCAACGCATGTTCTCTTGTGGGAGCAGGCATTCATACTGTCACCAAAGGCACGACTCCCCCTGAATTTCATCGGTCCCGCCATGACAACTGATGACAAAACATCCATTCGAACAAACCGGAGAGTGCAGGAGCGTAGGAGAAAATCATCCTTATTTTATATTGAGAGATCCAATATCAAGTGCGGAGGAACCCAAACCCTGATCCATATTCTAATCGGGTCGATCCGATCCCTACATAATCGAGTTTCGCTTAGTTGGGTCAACAGGTCGGGTCAGAATTTGCACCCTTAGCCCGGTTATCCAACTCCCTCTAGGCCCAACAACTTCACCTCCAAAAATGGAGGGAACATGCGCATCAGGTGCCCCTGCGCATTTTAGCACAGTTGCGTCCAATGTTACTTAAGAACTTTGA

SEQ ID NO: 127 - protein, 500 aa, unidentified
GACAGAGGAATTGGAGTTGATAGGACACGTGACGGTTTCACTTACCTGGAGACCGGCTCGAGATGACCGGGAAACAGGCTTCTTCTTATCCTTTTCCTTGCTGGCAGCAGTGGTTTTTCCCGCCAAAAGACCCTTCCCTCCTTTTCCCGCCATTTTTTACCTCTGAAAAATGCCAAACAAAAAATTAGAATATTCAAATTCCCGCCAAAAAACCAAAGCAAAGAAAAACAACATGACCCAAAAAAACAGATCTTCAAATGCATGAAAACCCAGAAATTAAAAAAAACAGAAAAGAAAAAAGACAGAGAACAGAGAGAGAGATGGGAAAATGGTAAGAGAACAGACACAGAAGACAGAGACTAAGATTTTCTCCTTTCAGTGTTTGGAGAACAATAAGATGTGATTTCAGTGAAAAGAAAACTCAGATGAAAGAAATGTGTAAAATAGAGAAACAGAGAGAGAGGGAGGGAGGGAGGGAGAGAGAAACCTGAGGTGTTTGG

SEQ ID NO: 128 - protein, 550 aa, unidentified
GAGGATATGATAGTGGAGGGGATGGACCATCCCTATGTGGGCCCCATGGTGAGTTACTTGAATCCTACTCACGGCAACCGTGTACCAACGCTTGGTAGCACCGTTGGTTGTGGCACTCCGTAACCACCCGGAGGTCTCAGCATCAAAGCCCGTTAATGCGTTGAAACCCCAGGCTATTATATAGCCCAACAAACCCGATGAGTAAAAACAGGGCGATGCCATCGCCATGAGGGGACTAGTCGGGTATGCAAAGAGGCCTTATACAACTCCGATAAAAAAAAACATCACGGCAAGCGTGTAATTAATTGACCACTCAGAGGATCCGGATTAAAACCCGTATAAACTATACAACCAGATGCGGGCGTAGTTCATAGGGATCTCCGCACCCACGCTACCTTTCCACTGTAGAAGCCAACGCTCTCTTTACTTCGTTTCCTCCGTCCACTTGACTCTACTACTTTTTTTACTACTACTCTACTCCTCAATTCCTCTGCTCCAATACCACAAAAATCCTTCTCTTTCCCTTCTTCTTCTTCGAATTCCTGTGGAG

SEQ ID NO: 129 - protein, 1991 aa, unidentified
TAAATTCCTACTGTTACAAACCGTGGTTGGACTTGTTAGTCAACCATAGTGACCAAAAGCAAAGGAAGGCTTTATCTTTTGCCTATAAAGGGACCAAATATACATAATTTTCTGAAAATTTAAAGATTTATAAAGTTGTAAAAAATGATTAAAATACAAGAAATCAGTGCGAACATCCAAAAATGACCATCGAATAATCTCTCTAATATGAAATGAATGAGAGAGAAGGAGGGAGAAAATAAATGCCCAACTACATCTCTATCTTATATCTTTCACCCTTCAAAACTTTACTAATCCTCATTCGGATTAAACTTGAATTCTAATATATTCCATTATGATGTAAAGCAAATTAGCCCTATTATAAATTTGCAAAAGGTAAAAGATTTATCAATAGCATAGAAAAGCTTTTAATAAAGCCCACCAAAATGGGCCCACAACTACAAGCCCGGCCCCACTCCACTCCCTTATGACTTACTTAGTGCGCTGGTCTGCTGCTTGCGGGGCAGTTCAAAACAAGTCCCACCTCGGCACTATCTGCCACTGCGACTCCAGATCTTCAACCCTCCAAAAACCCTAGAACGAGACGAACCAATCCAAGGTGGAAACCCTAGTTTTCGCCAAGCGAGCGATCCTACTCTCAATCTTTCCAAAGCAAAACCAGTAAGATCTCTGTTCTCGATTTCATCGAATCGTATTTGAGTGCGGATAAGAACAATCCATTTCGGGTATCTGCTTCAAACGAATCCACAGTGCCGCTGCCTGCAAGAGGAGCTTTATTCACATAAAGGTGAGCTTCATGAGCATCAGAAAGTCGCCTTCCGTTTGCTCATATGTCTTTTGATTTGTGAGGATTTGTATTATACCGTAAATGCAGCAGATTTAGGTTTTAATCGGTTTTTCTTCTTCTTCTTCTTTTGTGTTTTCTTCAGCATGCTTGCCACGTTAGATTCTAAGGTGCTGTAAACAGATTTGAATGCTATAAATTTTTAGTCTTTTCTTTTACTCTGTGTGAAGTTGAGATTTTGTAATATTTTCTTTCATCTTTGAGACTTCTTAGGGTTTCCTTACAGACTATGTGCATTTGAGCTTCTTCTTGGATTTCTCTATTCTTCCTATATCTTGTGAGGAAGTTTAGTGTAAAACCCAGCTACTGTATGTTTAGGGTGTCATTGGGTGTAGCATTCCCTCATCCGCACATTGCTATTCTAGAGGGGGAAAAGACTGAAGAAACAGTTGCTACTCGCTATGATGATGTGGCTGCTAGTGGTACTAAAAGATTTCATACAATAACAATGCCTGGAGGAAAATTCTGTTATTCTACCTAACAAGTGTCCCAATAAAACCAAAATCAGTGTCTTCACGAAACGGAATCCACTGTTCTATCATGAGATACACTGCTTTTTATCATTTGAGGGCGTACCCGACACCATTTATTGGTTGTTAGGCATGTTTGTTTGCAAGAATTACTCTTATAGGTTTAAGAATTCCGCAAATGTTGTACCCCACCTGGTTTCACAAATGCTCAAGTACATTGTATCATTTTAGAAACCGGAATTTGGTGAATTTTACGGGCCAAACGTGACATTTTGCAGGGCTGGTTTTGTGGATTCTCACCATTCAAACAAGGAATTCAGAATTCCAGAGGGACAACAGCTTTCATTGCTGTCAAGAATTCCCAACATAGGGAATTCTCATCAGAAAGATCTCCAAACAAACGGTTAGTTTTCTTGAAAGGATATACGTTCATTTCGGTCTAGTATTTCTGAGATCTAAGCTTTGCCTTACATTGTAAGATATCTTTCAAGTTGTTCTAAGAATTTTACTCTAAAATGCTGCAAATTCAGATTCTTTTTACACTTCTCTTACTGTTGATGAATAAATATGTTTATCAACATATTTGAAAGGCATAAATAAACTAAGCAGTGATTCTTCGATACCATTGACAGATTGTGGCCTATATTCGTCTGAATTTAATATACCCAAAAGTGGTA

SEQ ID NO: 130 - protein, 679 aa, unidentified
TCAACATTGAAAATTTGAGTCGTCTGCTGTTATTTCAAGGTTTAAGAAAATTATCCATGTGAAAATCATTTCAAATGATACTTTCATGCCTCACAAGTGAGTAAAATTCTTTTCTTTCTTACCAAGATATCAAATACAAACTCCGCACCAATACAAAATCCAAGGTCCTCTATTTGATGAAAATGAAGATTTGCTGAAACAGTTTAACAATTTTAAGTGAATAATATGAGAAGAGAACGAAACGTAGTAGCTCATAAATTAATGAGGCATGCCCAATATACCCCAACCCTGATTAAATAATATATATATAAATATTGATATGTTGGATTCTAAATAGAAAATTTAGAAAGACTTCTCACGGAAAAAAAATCTCCTTTCCCCAAACTCCAAGCCAATCAAATACTCAAATTCCAAACCAAACTTAAAGTGGGTCCTACCCAACAATTAATGAAACCCAAAAAAAAAAAAAACCTAAGCATACTCTCCCACTCTTCCTCCATCTTAGAAGAAAGTCCATAAAATTGTCCCAACAACATAATAAAAGTAAGCAAACTCCAATTTCTCCTTTTTCATCTTTTTCATTGTTTGTATCGACACCCTACTCTTTAATACGTGCACCCGCGCACCACCGAGAGAAACGAAGAGAGGGAGAGAGAGAGTAAAAGAGGGGTGTAGAGAG

SEQ ID NO: 131 - protein, 1305 aa, unidentified
TGTATATAAAAATTCAGGACTAATTTTTTCATAAAAACCCCTAAAATAATCATCATCTTATAGTCCTAATCACATGACCCAAAAATAAAAATAAAAAATCTTATCCCATTAATTGTTGAGCCCAAACCAGGCAGCTGTTCTTTGACCCTTGACCCTTGACCCTTCCCCTCCAACCATCAGAAACAAGCCAACAGGGTGGGCCCCAAAAACCAGCGTGTCTAATTTGATAGTCACCCAACGAAATTTCCAGTGTCACCCCCCGCAATAACAATATTCTAATTCCTCTAATGGCCAGGCAGTGGCCCTCATTATCCATCTCATCCCAGCTGGCAGTCTCTAGTCTCCACCACATCATGCCACTCCTGTAATTTCATTCTCTTCCAGGGGCATTTCCCAAATCCACAGATGCAACGAAGTTGGGAGAACCAGACACACGACGCAAGGTGGAGTTTCCTCTCCCTACATATAAAAGGGCGGGGAGGGCTCTCGCTCTCATTCGCACCGCTCTTCCCGCAAAGATCACCAAAGCCCGAAAACGCCCACGTAGAAAGCCCTAGAAACGAAAAGCACAGATTGAATAGAAAAACCCAAGCAGAGAGAGAGAGAGAGAGAGAGAGGAAAAAACCCTAGAAAAAGGAGGATTCGTTGGCTTGATATGTGGAATCTTTGCTGCCACTGAAAGACCAGCTCCATTTGTCTGGATGTTCGTCTTGATCTCTCTCTGCAGAAGGTATGTGAATGTCTCTGCAATCTGATTGGGTTTTGTTAAGTTTATGTCATTTTCTAGAAGTTTGATTGTGCTGGCAAGTTTTGATTTTTTTTGAAAATATTGGGTTTGTTGGAGAGATGATTGGGGTTTTTATTGGTTTATTGTGGATCTGGTGAATCTCTGTTTTAGATGATGGGGTGTTTCAGATGATAGGAAAATGCTGTTTCTTGGGATGAAAGTTAGATCTGGATGGGGTTTTTTTTCTTGAATTCTGAGGGTTTTGAGGCTTTTAGCTTTGACTACGCGTCCGTGGAATCTCATTCTTTTTTATGTATTTTTTTTTAAAATTTGGGTTTGAGCTTGAGATCTGTGTCTGATGGTGAGATTTTTGTCGTGATTGGTGTGAAAGATTGGATTTTTGAGGCTGCTTTTTTGTTCATTTGTGTTTGAATTTTGAATTTGCAGAATCTGAATCTGTTTGGAAGATCAAGATCAAGATTCGATTTTTGCTGACGGGTTTTCTGCTGTCGAAGAGCTTCTGGGTTCTCATTTCTGTCTGATTTGGTGGTCTTCCACCTCATTTTTTTTTCTCCATC

SEQ ID NO: 132 - protein, 562 aa, unidentified
TGCGGAACAAGGACTCTCATGGTCTCCACTTGTCAATGACCCAAATGCCCCTGCACAATTCAAATTATTATCTGAAGAAAGACGAAACTCAAATTCCATTAGAAGTCATTTTCCTTTAGCATATGCTAACATGAAAATGTCAGTTACACATATAGACATTGACTACGGATAATTTACATATGTAAAGTACAATATTATGCTTGTTTGGTAAACGATGAATGATTCGGATTACATTTGTAAGATCTTCTTCATTTATATCAAGATAAAGATTTAAGATATGAAATTGTATGTTTTTAGATTTAATGAGTCAATAACAATCTGAACAGTTGAAATTAAAATTTTAAGCTCACAAGGGTATCAAGTGTGTAATGGTGAATAACGTGATCACATGTGACTGTTATGCGTGACCATAATTTACGTACGTAAATGAAAGTAATAATGGCATTTCATGTAAATTAATTAAATCCCAAGGGTAGCTCATCCTTTCACACGTTTATAAATAAACAGTGAGATCCTCCTTCCCCATCATCAAATTAAACAAAACATCCCCCACCAGCTCCCA

SEQ ID NO: 133 - protein, 779 aa, unidentified
TTTATATTACATAAGTAATAGATTCATTTTTTTTAAATCAAAATTATTTAAAAAAACATGGCTCTTCCAAGTCTTTCCATTGTCTCTATCCGAGTCAATTTTTTCGCATTTAACGAGTTGAGTATAAGTTACGAGTTTTCCAACCTTGATTCTTATCCACTTTTATTTTATTTATTTATTTTTAATTTACCATACGAAACTTTATTTCACTTCACCCCTATGCCAACACAAAAAAAACACAAAAAAACAGGGACCAAGTATGTGGCATCTGATCAAAGACACGTCAACCGTCCAGGTAGAAACCAATTTCAGAGAATTAACATTCCTCTGTGGTTCCCCACTGAAAAGGGCCTGGAAGACTGATGAGAGACGTCACGCATGGACGACCATGGGAAGCAAATGTAGGGTTTCCTGTGGCAGAGGACTGCAAAATCAAACACCTGCTTTATATATATATATATATATATATATATATATATATATATAATAAAGCGAACTGTCTTCTCCAAGGGTCCAAGTCAAAACCAAACCAAAGCGTTGAGACCAGCTGCATCTCCCTCTCTCTATATTTTCATAGCTTTGGCCACCGAAATCTCTCCAACCAAAATCGAGAATTTCTTCTTCTTCTACTCTTCTCTTCAGATCTTTAATCAATCTCCAACTCCATATAAGAAGAAGAAGAAGAAGAAGTAGAAGTAGAATCGATCTTCAGATCTCAGAATCGATATACGAAGAGGGAAAAGAATTGATCTTTGTCTGCGGAACGAGAAGCAGTGGAA

SEQ ID NO: 134 - protein, 500 aa, unidentified
ATTAATTAACAAAGACCGGTAATTTTAATAGAGGGGCTAACCTGTTAGTGGCCCTAGAGCAACTGCAATCTGCTCTTCTCCAAAACGAGTGAAAGGGGAAATCTATCTTAGTAAATTTGCATCTAAGACGCTCCTTTTATAGGCAAAAATTTCAATCAATAGTCAATAGATGCAGGTAGTAATTGCAGTTGGAAGAACTTACCCACTCTCTGCCTTTGCAATCGATTCAAGCGGTCTTATGTGATGGTTTTGGGAGAGGACGATTTGCCCCAAGATCTTCTTGAAAAATGAACCCTCATGTGAAATGCAGATTTCTAGGGCATGGGCGACATGTACGAGTGGCTAAGATTAATCGAACTCACCTATTCAGATCTGCGATTTAAATCTGGCGAATCTGAGGCAGGCGCAATATGCACGGCATTGGCTGAGTAGAAGAAGGTTGCAGCGACTCCTCTTGATGGGCATTTACATTCGTTGAGAAAGAAGGAGGTAGCGTAGGG

SEQ ID NO: 135 - protein, 816 aa, unidentified
CCCAGGGGCTAACTAATTCCTAAGTTGGTTTCCGCAATAGCAATGTGCAGAGTTTCCACTCAGCTTTTGTAATTAGGAAAAGGGTATAAAAAAATTCCCTTCGTTTTTCACTAGATTAGGGACACCAACACCATCAAACAACCAAATCCAGACCACCCAATGGGCCCATTGAAAAGCATAGCCACAATGATACATTGATAGGATGATCATAATCACCAACCAGTGTCCATCAAAACACCTGCTCCTGAATGGACAAAATTATAAGTGTTAACTTGGGCTGGACTCCTCAACACAGTGGGGTCCAAGATTTTAATGTGTACAGATGCACCATGTATGGTGGATAAAGTGCCACATTGTTCTGATGTGCAGTTAAACTATACTTAACATCTATGAGAAATAAGTGAAGTCCAACGCCTAGATTTTGACTGTGTACATGTTAGAGAGAGACTCTGAGTTCGAGTGTCGTTTTCCAAGATATACCGCCGAGGAGATGAGGGAGATAGATAACCAAATTCTACTTCTTTCGATACCTGGCACTTCCCTCTTATTTCCTCTTTTTGCTGTGACAAACATACATCTCTTCTCCTCTTTCTTCACCCATCTCTCTCTCTCTCCTCATCTCTTGGGGATGTCTGTTTCCTGAGGTAGATATTAGTCTTCCTCTACTCCTCTTCTCGCCTTCTCCTACACCCTCTCCTTTCTACCTTGGAGCATACCCATTTCTCTCTCTCTCCCTCTCTAATTGATAAGTTTTTATAATTTCCTGGGCACCCAATTGCGCAAAAGGGAAGTTCTTCCACTACAACTGCTCAAAAA

SEQ ID NO: 136 - protein, 500 aa, unidentified
TTTCAATCAGAACAGCAGTGGAAAGGTCCATTTTTATGTTATTTTTACGTATTTTTTGCCATTTTTCTGAATTTTTTCTCGCCTAGGAACGAATAGAGTTTCCGATATGTACAGACTTTCAGGTCCGAACAAACTTACTCTGTTCGAATTTTTATAGATGTGTATAAGAGACAGACATATGATGCATGAGCATAGTTTTTTTGAAAACAAATTAGNNNNNNNNNNNNNNNNNNNNNTTTGATACCGAAAATGAATTTGGTACTTAACAATTAAAGATGACAAGGTATAATGTCATGAAAATCTCCCCACTGTGGACCTGAAAATCTCCCTAGTTGGAGGATCTTAGCATCGCATCCTTTGGCTGTTTTCCTAGACTTTTGCCTTATTATTGGACTCTAATTTAAGTGTCAGGATCTTGTATTCTTTGATTTTGTATGATGTGCTTCATACATGGTAAGTTATGACATTAACAATCTAGATTATCAACCCATTGGCTCTAC

SEQ ID NO: 137 - protein, 634 aa, unidentified
GATGGGAAAACATTGGTCACCCCATTGAATTGGGTGGAAGAAAAAGAAGAACACAACAATTCTTTTGAATTAATCAAACCTTTTGACACTTCACTAGTTTAGTAAAAGTCAAGTAAAACCTCCATTTTTCAGATAAGGGGTCACATCAAAAGTGCACTTTTCCAAAATTTTACTTTAAAAAAAATAAAATAAATAAAAAAGCAACCTTCTTTCTTCACTAAAAGACAAAAAAAAAAAAAACCCTACAAATTTTCTATACAAAAAGTAAAAAAGATTCTATACCAAACAATTAAAAATAAAATATCATTATAACCCACTTATATTTTATTTTCTTTTGATTTGATACTTTGCTTTATCTTTATTTTTAAGTAAAATTACCAAACAAGTCCTCTAAAGAGTGAAAGAGGATAAAATTGAGTGGATTTTACTTTTTGATAATTCCGATGAACATTTCCCTCTATTTATATCTCATTACTTCTTTCGATATGATCGAGGCCGTTTGGACAGCTACCAAAAACCCCCTCCCACGCCGCGTGCACGTGCGGCATGCGACCGAATCATACATAACCATTTCGCAGAAGGAATACGTAGGAGCGAAAGCAAGGTCTCTGCAGAAGCTCTCTTTCTCTCAATC

SEQ ID NO: 138 - DNA, 671 bp, unidentified
  source 1..671: mol_type = other DNA
tcacagcaaatcaagaagacaggaactgatggctacgaatttgaggtaaactttgaaagaatccagctgctatgtgctggcattaaatccttctaaatctgaactgctctaaataagtataaccctcacagactaatttgtgctgaaactcacacacgcaatgggaatgctccctattttgggtaggatccattgataacagggcaatcacttatcagggtccatccatggacatccctatcggccatgtatgccatctagagtccaattaatccggaccatccaattcagaacccaacttagatttcagatgcaagcagcataaatccactaatggccccactcttagtgggcaatgatcacatgacactcgcctttcaccatattttactcacatgtattagatgcttccatgttttacgcgcactacacgctcctcctatattttatacctacctcaaatccaaagcttttcaatttgaactcctccctcgttatctttccaaagaaagcagatcccaagccccccgccccccgcgccccccgccgccgcccccccacccaaaacaataccatcccattccacgagtattgatttctccgccctcttcccttttatacatcgccctacatccaaatatcccaccccacaaagaaacaaagaaaagaca

SEQ ID NO: 139 - protein, 500 aa, unidentified
TAATAATAATAATAAAGCAAATAATTTATATATATAAATAATCAAAACGTTACAAGATTGACTAGTGGCCAATGAAGAGAATTTTTGGCTCCAACAGAAAGGAAAGAGTTCAATAAAGATTTCTTCATTTCTAGCTTTCCATATGATGTATTATGGAGAAGAAAACAGCCTCCTTGCCTATTCTGGTAGCTTTATCAGGGTTGGCAGAGTGAGCTTCCCATATACTCTTTGTAGCGAACACGTAAAAAAGCATAAAAAAATCACAACAACATCAAACAAAACATGGGGAAAATGTGAATTACTGCCCCACAGTGGGCCCTAACAGACACGTGTCTCCAATCCATCTCACATCCTCTCCTATAAAATCCCATCCAACAAAAGCTCCCACACACACTCTTCTTTAAGTTCTAAGTTCTAATTTCTAAACTAGCTACATATCTATTTAATTTCCACCAACCCCACCCTGCTTTCACCATTTAACAGTCACCACCACCCCCACA

SEQ ID NO: 140 - protein, 500 aa, unidentified
ATAGGACTTAGAGCTAAAGAATTATTTATTTCAGTTTAATTTTAAATTATGATAAGATTTCTTATTAGTACAAAACTAGTATCTATCAGCAAATCAATTGTTTTGCTAGAATTTTGGAGAGGGGAATGCATTATCTGTAAATTAAATTAAAAAAATATTTTGTTCAAAGAATTTTTTCCCTATAAACCTTTCCAATAAAAAGAAGTCCCATTTCTCTCAAGTCCAATTCCATGACCCCATTATCAAGAAGCCCCATTATCTGACTTTTTGTGACCATCGAAAGCAAACACTAGCACTCATCTGCAAAATTTCCATCAAATCCGTCAAAACGGCCGTTAACTCCCCCGTGCCGTACAGGAATCCCCCGTCGGCAGCAAAAACACTCCCCTTTCTCCCATATCCTCCCACCATTCCCATCCAGCCGCCCCCACCCCCGGACCCTCCCCCTCCGTCAAAATCTCCGTTGCCCTAACTCCGTCATCACCTCCGTTAACTCCGCC

SEQ ID NO: 141 - protein, 820 aa, unidentified
ATTTTTATTCAGTTAATATTTCCTTTTTTGGCTCGCACTCTTTGCTTGCTGCAATCTTGCCTACCTAGTTAATGATATTTTTTTTTTCCAATGAGGTGGGGCATCTCCAAGATCCAATAGGATTATTCCCTTGGGCGTGCCCAGTAGCCCCAAACAAAGGCAAATCTAATCAAGGGCAGACTAGAAAATTGGACTTGAGGGCTCCATGTAAAAAAACATAAGGAAAAATGGCAAATATAAAAAAAAAAATACAAATTAATTCTCAAAATACGCAGCGGGTTCAAATATGGCTTCAGCTACAACCCCTTCTACTTGGGTTCGCCCCTAGATCTAAATGCGGGTTGTGATCACAAGAAATGAGACCAAACCCAAGCGACCCCATAGATAATTTGATCAGTTTTGGGTTCTTCTCCCGTATGTAAAACATGTGGACCCCACCGGGCCGAAAAGTGGATGTGTTTAGGCCGTTGATTGATATCTCGCGGCAGTTGGCGCTCGAAGTCGACTCCCAATTCCAAAATTTCCACGAGGCACTGTTGCTGGAGCGCATTGCATTGCAATTGCAAGCCCCAAAAAACGATGAAATACGGCGCATTTTAGCAGGGCGTGAGTATAGATAGTTGGGAGAGGTGGGGGGAGAAACTTATGAGAGAGGCGGAAATCTCGATGCTCAGGAGACATTACAAAACCACTCTCACTCTCCCTTCCAACGACTTTTCTCTATAAATCCCTCCTCCCATCTCTCGTTCCCATCTTTCTTTCTTTCTCTCTCCAGCTCCAGCTCCAGCTCTGCAAATTTCTCTGTTCTTCATCTTCTTCA

SEQ ID NO: 142 - DNA, 729 bp, unidentified
  source 1..729: mol_type = other DNA
ctattataaaaggatctaataaatagtaccaagtgatgtgccatgcaatgattcactagtgagagatttttttttcttcgaatctttccaatccattttgtggagccaactacactcaccataatccacaagcggaatctttggaaagaaaaaaaaagaaaaaaaaagaaaaggaaattgcatgttgattgcttgtatgtgggactttaattagatccaacaattataaccattgatccagaacctccaaataaacaggctacagtgtggcttccctgtttgaagaccactaactttatatagttatcatgatcaaatgaagctatttttaagttgcagttgataacctactaatagaaaacccatgggtatccaaagacgggaaatataaactctcaaccaggacccacaaggtgggctgcaccagtgccaaactagttgcacttgatcatttctcaagaaacaagaaaagaaacccaccacccatagagctttggagactgcgaccttacacacaactctcaaagctcccctcctttttctattgtctaatacacattacactgctggtgctctgctcattccgcaaaactaaacccacagcaacacatgctcctcgatgccttaaacccttcttttatctcctcttccgactcctcttcttcctcccccacttctccccacttcccctactagtcctccaccactttcttacaaacaataacaaag

SEQ ID NO: 143 - DNA, 500 bp, unidentified
  source 1..500: mol_type = other DNA
tcaacccaatctcttattcttttcttacactaggccacacaagactttcgcaagtgtatgggcttaaaacacctccaatcgttgatgccaaacctgcattatttaattttattaaaaaacaaaggtgtagcctagagataggcacatgccatcaaaagagggtgccacgatgccttgcacaaaagcttttgcttaaataattgttattagttcattactgcttgatgaacaataaattctgacactgggcacaacataataggtcacctctgtgcaatgcagttatctaataggcatcacgtggcacatgcctttccatttgcaattaaaaaaaagtttttttttttttttggacactactgttataatatatggcatgtagatgtagattctacttagcttttccaaataaagaaaaaggaaaaagcacaagaattattatattgaattatgttacagttgattccatgaaatgtaggtcacctctgctttgggtttct

SEQ ID NO: 144 - protein, 943 aa, Persea americana
ATTTTAGATACAATAAATATTGGTTGTTAAAAGTTTTTGCTTTATTTCTAAAAATAAAAAACACAAGTTTCAAAACTTTTGAAATTAATTCCTAATTTTCCCCCATTTTAAAGTGATTTTCCGATGACTTTTTTTCTCAATATTCTTAAACTACTAATATTACTTTTCATTAATAAAATAAAACCCTCTTTCAGTTACTTTATAATCCCCTCATTTTCTTCATTTACGACCTTACGGTAATTGAGGACAACTAGTATCCCTACAAAGATTTGCCGAATATGGTAGTTTTCATACCTATATCACAACTTTAAATGAAGCTTAATTTAGATATTTAGAAATAAAAAACCCGAGACAAAGTGTTATAAAAAAAATACCTTCAATCACCGTATGTCCTTAATTGAAATAAAATAAAAATTTAATGCCTCATCTGAAACTTTAACTTTCCCAATTAAAAAAAAAATCTGGAGTGTGAAGATCACTTTCTTCTCGCGAGAGGAACTCCCCTCGTCATTTCTTCTTTTTCCTTCCCTACTTTCTAGTTCTCTCTCTCTCTCTCTCTCATTGAAAACTAAAATATAATTACAGTCCAAAACCAGAACAAAAACTTGGAGAGAGACCCGTGAACGTGAAAAAGGAGGTTTGCCACCTGTTTTGGAATTGCAACTTCTCTACATTCGGTCTCCTCTTCCCTCCTTTCCTCCCTCTTACCATCTCTCTATTTAAGTATCCAAGTATGGCTTCCCCTTCTCCCACAAGAATCTTTCCCTCTCTTCTGCTTCTTCTTCTTCTCCTTCAATGAGTTCTATTTCTCGATCTTCTTTCCCACTGAAATTTCGCTTTGATCAGGACTCAAATCACATGCTAGATCTCCGAACTCCTCCTCCTACTGCCGCTTCGATTCCTGCAAAAAACCTCCGATTTGAAGCTCAGATCGTCCAACGGA

SEQ ID NO: 145 - protein, 1434 aa, Persea americana
GCGGGCCCTGAAGCGCTTGTTTATTTAAAAGGAGAACTCATATGAGTGATGACATGGCACTCTCCACATCCCTCCTCTCCCTTTGCTTTAATATATATAATATAATATAATGAGTGAGATCCGGTAAAGGCAAGTGCTGTCGCGGTGTAGGTAGGGTTGTAAACAGGTCGGTTCTAGTACCATGGACCCGGAATTGGAACCATTAAAGAGAAGGTGGAACCGGATTTGGACCCGAAATCATTTCGGAACCGGTTTCTTGGCTCTGAAACCAGAACCAAAACCATATATGGGATGGGTCTGGATCCGGATCCGGGTCTAAAAAGAGAATGATATTAAAAAAATGTGGTTTTTAAGAGATATTTTCAAAAATTAAGGTAATATATCGGGTCTCGGGTCCATGGGTCCGAACCAAGGTGGACCCGGACCCGGACCCATTTATTTATCAAAGCCTAAATAAGAACTCAGACCCAATTTTTAATGGGCAGAACCTCCTGTGTGCTCACAGAATTGGGGACTCATTTACATCCCGAGCCTTAGGTGAGCCAAGAACAGCGCCCAGACAGAAACGAAATCTGTCGCATAAGAAGTATCGAAACACCTACCTTTGATCTCTTTCTTCCCCTTCAAAAACGAGGCTTCTGACGATCTCATCGATGCTCTCTCAGCAACTCCTCTCTCCTTTTATATTTATACTCTCTCTCTTCAGCTGCTTCTATTCATTCCCCAATTGCTTCTCCTCCTCCTCCTCCTAGGGTTTCCCTCGTCCGGATCACAATCATCATCTCCTTCGCCATTACCAAAAGATCGATAGCTCTTCCTACGATCTCTGCGTTTGTTAGGTAATTTCGCTTCGGCAATCTCTCTCTGTTCTTGTTTTTTCTTCTTCTTTCAGATCTTATGCGCTTTCTGGAGATCTGGAGCTCTGTCTTGGTGAATTTTTGAGCTGTTCTTTTCGGATCTTGTTGAATTTGTTTTTGCATCTTGACATCTCGAGCTCTCGTTCTTTCTTGAGATCTTTTTGTGAACTTTTTCTTCTCTTTCTGAGCTGTTTGTGTCAGTCTGTTCGATTCGATTCTGAGATTTTGTTCTTATTTGAAAGACAAATCGAAGTTTCCGATTCTTCTTGCTGTTTGAAAAAAGTCAGTTTTCCCATAATCTGTAGTCGTTTTTCATGAATTAGTTCTTCGTGATTCTAAGATTTTCCACTCCAGTTTTGACGAAAAGTCAAAGTTTCTGTCCTTTTCTGCTCTTTTCTTCGGGATTCGGAAAGTCCTGTTTTCTCCGTCATTTTCGGTACTGTAAAGCCATTTCCTTTGAATTGCCGCAATCTCGATTCATTTCTAAGTGTATTTGTTTTTTTTTTTTGGTTTTAAGGTCTTTCGACTTCGTTTTGGAGGGGTTTTTGCTGAGCTGAATCAGGAGAGGAGATTGAAT

SEQ ID NO: 146 - protein, 685 aa, Persea americana
CTCATTAGGACAAGCAGCAACAACAACAACAATACTATAAGTTATAAGATTTTATATCAATTAAATGTTCTATTGGTTAATAGGCTAACCAGTGGATCCAATATCGAATTGACCCAATCAGATCCGGTAAACACTCGTGGATTTGGATGTGACTCGGCTCTAATCCAGACCCAATACCCAACAAAAAACATAATCAGATCCAAGTCCAGTTCTCCTTACCTGACCCAGTTCTTATTTTAACAATGGGAATCCGGTACTCCTCTGGAACACTTGAAACGATGCTCGTCACTACAATTTGGTGTAACTCAAAAGGGTGCACCTAGTCACCTTCATGTAAGGACCCTTAAACCCTATAAGAGAAACAGATCGAGCTCAAAGCATGCAAGAGAAATCAAAGAGGAAATCTAGAGAGAGAAGAGAGAAGAGAGAGGAGAGAGAAAGAGAGGGGACAAGAGAACAGAGACGACGATAGAGCCGCTCGTAGACGGCCAGAAAGCACCCAAGCGCCGGCCCACTATCATCATTTGCCAATCACTACCGTCCAAAAGCCAACTTCTCTTCTCCTCATTATATCTCTCCTTCCTGAAACGACCCATTTGTCTCTACTATTCTCTTCTTCTTCTTCTTCTTCTTCTACTTCTACTTCTACTTCTTCTTCCATAGTGGGTTGATTAGGAGTGCTCTA

SEQ ID NO: 147 - protein, 517 aa, unidentified
CCAACAAAAGGATCGGTAAGGAGGTGAGCGTTTCATGATAAAAGGTAGAGCACCTTTTATCATGAAACGCTCACCTCTTTACTTGATAGAATATTCATGTCTCATCTTCAATCCTTTCATTGCTAATAAAGGAGAGATTATTTGACGGTCATTTTTTAATCATCTTAAGCAATACAAGTCTCAGAATCTCTAAGAATTCAATTACATGAAAGAGTAAAGATGCTTGGAAAGTTTTACTCTCACAAGTGAAAACCAGCTTGGCTCATCGCTGGCCAATGGAGGAGAGAGATGACAGAAATGTATAGATGAACAAAAGGACAGGAGACTAGGGACGAGGCCGTTTGGATTCGGTCACAATGTAAAACTGTCTCACTAGCCGGCCTTAGCTGTTTTGCCTTCTAAAACCAGCTTTGCCGCCCTTCATTTATTTCTTACACCCTTTATAAGGCCCCACCACCAATGACCAAGTCGTCTCTGCCACTACTCTCCCTCTCTCCTTTCTCTTAACAAAGAGCAA

SEQ ID NO: 148 - protein, 172 aa, Tomato bushy stunt virus
MERAIQGNDAREQANSERWDGGSGGTTSPFKLPDESPSWTEWRLHNDETNSNQDNPLGFKESWGFGKVVFKRYLRYDRTEASLHRVLGSWTGDSVNYAASRFFGFDQIGCTYSIRFRGVSITVSGGSRTLQHLCEMAIRSKQELLQLAPIEVESNVSRGCPEGTETFEKESE